SEQUENCE LISTING (56 sequences)

SEQ ID NO: 1 - protein, 388 aa, Oryza sativa
MGAGGRMTEKEREEQQKLLGRAGNGAAVQRSPTDKPPFTLGQIKKAIPPHCFQRSVIKSFSYVVHDLVIVAALLYFALVMIPVLPSGMEFAAWPLYWIAQGCVLTGVWVIAHECGHHAFSDYSVLDDIVGLVLHSSLLVPYFSWKYSHRRHHSNTGSLERDEVFVPKQKSAMAWYTPYVYHNPIGRLVHIFVQLTLGWPLYLAFNVSGRPYPRFACHFDPYGPIYNDRERVQIFISDVGVVSAGLALFKLSSAFGFWWVVRVYGVPLLIVNAWLVLITYLQHTHPALPHYDSSEWDWLRGALATVDRDYGILNKVFHNITDTHVAHHLFSTMPHYHAMEATKAIRPILGEYYQFDPTPVAKATWREAKECIYVEPEDNKGVFWYNNKF

SEQ ID NO: 2 - protein, 387 aa, Hordeum vulgare
MGAGGRMTEKEREKQEQLGRAGGGAAFQRSPTDKPPFTLGQIKKAIPPHCFQRSIIKSFSYVVHDLVIIAALLYAALVWIPTLPTVLQLGAWPLYWIVQGCVMTGVWVIAHECGHHAFSDYSLLDDTVGLVLHSWLLVPYFSWKYSHRRHHSNTGSLERDEVFVPKQKEALAWYTPYIYNNPIGRLVHIVVQLTLGWPLYLALNASGRPYPRFACHFDPYGPIYNDRERAQIFISDVGVLAVSLALLKLVSSFGFWWVVRVYGVPLLIVNAWLVLITYLQHTHPALPHYDSTEWDWLRGALATMDRDYGILNRVFHNITDTHVAHHLFSNMPHYHAMEATKAIKPILGEYYQFDGTPVAKATWREAKECIYVEPEDRKGVFWYSNKF

SEQ ID NO: 3 - protein, 387 aa, Zea mays
MGAGGRMTEKEREKQEQLARATGGAAMQRSPVEKPPFTLGQIKKAIPPHCFERSVLKSFSYVVHDLVIAAALLYFALAIIPALPSPLRYAAWPLYWIAQGCVCTGVWVIAHECGHHAFSDYSLLDDVVGLVLHSSLMVPYFSWKYSHRRHHSNTGSLERDEVFVPKKKEALPWYTPYVYNNPVGRLVHIVVQLTLGWPLYLATNASGRPYPRFACHFDPYGPIYNDRERAQIFVSDAGVVAVAFGLYKLAAAFGVWWVVRVYAVPLLIVNAWLVLITYLQHTHPSLPHYDSSEWDWLRGALATMDRDYGILNRVFHNITDTHVAHHLFSTMPHYHAMEATKAIRPILGDYYHFDPTPVAKATWREARECIYVEPEDRKGVFWYNKKF

SEQ ID NO: 4 - protein, 387 aa, Brachypodium distachyon
MGAGGRMTEKEREKQELLSRASGGAAVQRSPTDKPPFTLGQIKKAIPPHCFERSVIKSFSYVVQDLVIVAALLYVALVWIPTLPSALQLGAWPLYWIAQGCVMTGVWVIAHECGHHAFSDYSLLDDIVGLVLHSWLLVPYFSWKYSHRRHHSNTGSLERDEVFVPKQKEALAWYTPYIYNNPIGRLVHIVVQLTLGWPLYLAMNASGRPYPRFACHFDPYGPIYNDRERAQIFVSDVGMVAVSLALLKLASSFGFWWVVRVYGVPLLIVNAWLVLITYLQHTHPALPHYDSTEWDWLRGALATMDRDYGILNRVFHNITDTHVAHHLFSTMPHYHAMEATKAIRPILGEYYQFDPTPVAKATWREAKECIYVEPADRKGVFWYSNKF

SEQ ID NO: 5 - protein, 384 aa, Brassica napus
MGAGGRMQVSPPSKKSETDNIKRVPCETPPFTVGELKKAIPPHCFKRSIPRSFSYLIWDIIIASCFYYVATTYFPLLPHPLSYFAWPLYWACQGCVLTGVWVIAHECGHHAFSDYQWLDDTVGLIFHSFLLVPYFSWKYSHRRHHSNTGSLERDEVFVPKKKSDIKWYGKYLNNPLGRTVMLTVQFTLGWPLYLAFNVSGRPYDGGFACHFHPNAPIYNDRERLQIYISDAGILAVCYGLYRYAAVQGVASMVCFYGVPLLIVNGFLVLITYLQHTHPSLPHYDSSEWDWLRGALATVDRDYGILNKVFHNITDTHVAHHLFSTMPHYHAMEATKAIKPILGEYYQFDGTPVVKAMWREAKECIYVEPDRQGEKKGVFWYNNKL

SEQ ID NO: 6 - protein, 383 aa, Glycine max
MGAGGRTDVPPANRKSEVDPLKRVPFEKPQFSLSQIKKAIPPHCFQRSVLRSFSYVVYDLTIAFCLYYVATHYFHLLPGPLSFRGMAIYWAVQGCILTGVWVIAHECGHHAFSDYQLLDDIVGLILHSALLVPYFSWKYSHRRHHSNTGSLERDEVFVPKQKSCIKWYSKYLNNPPGRVLTLAVTLTLGWPLYLALNVSGRPYDRFACHYDPYGPIYSDRERLQIYISDAGVLAVVYGLFRLAMAKGLAWVVCVYGVPLLVVNGFLVLITFLQHTHPALPHYTSSEWDWLRGALATVDRDYGILNKVFHNITDTHVAHHLFSTMPHYHAMEATKAIKPILGEYYRFDETPFVKAMWREARECIYVEPDQSTESKGVFWYNNKL

SEQ ID NO: 7 - protein, 383 aa, Carthamus tinctorius
MGAGGRMSNPSEGEKKTELEGIQRVPYQKPPFTVGDVKKAIPPHCFNRSVIRSFSYVVYDLTIASILYYIATTFIPLLPHPLAYVAWPIYWAVQGCVMTGVWVIAHECGHHAFSDYQWLDDTVGLILHSVLLVPYFSWKYSHRRHHSNTGSIEHDEVFVPKLKSGVRSTAKYLNNPPGRILTLLVTLTLGWPLYLMFNVSGRYYDRFACHFDPNSPIYSNRERAQIFISDAGIFAVLYGLYRLAAVKGLVWVLTVYAGPLLVVNGFLVLITFLQHTHPSLPHYDSTEWDWLRGALATIDRDYGILNKVFHNITDTHVTHHLFSTMPHYHAMEATKAIIPILGDYYQFDGTSVFKAMYRETKECIYVDKDEEVKDGVYWYRNKI

SEQ ID NO: 8 - protein, 383 aa, Olea europaea
MGAGGRMSIPPEGKKSKSDVIQRVPQSKPQFSLGDIKKAIPPHCFQRSVLRSFSYVVYDLTIASLLCYVASTYIQFLPNPLSYFGWLLYWICQGCVLTGVWVVAHECGHHAFSDYQWLDDTVGLILHSSLLVPYFSWKYSHRRHHSNTGSLERDEVFVPRVKSGIRWISKNLNNPPGRVLLLVFQLTLGWPLYLMFNVSGRHYDRFACHYNPHSPMYSDRERAQILISDAGVLAVTCGLYRLTAAKGLAWVFCVYGGPLLVVNGFLVLITYLQHTHPSLPHYDSSEWDWLRGALATVDRDWGILNKVFHNITDTHVSHHLFSTMPHYHAMEATKAIKPILGNYYQFDGTPIFKAMWREAKECIYVERDEGDQNKGVFWYNNTL

SEQ ID NO: 9 - protein, 387 aa, Setaria italica
MGAGGRMTEKEREKQELLGRAAGGAAMQRSPVDKPPFTLGQIKKAIPPHCFERSVIKSFSYVVYDLVIVAALLYFALVFIPALPSPLHFAAWPLYWIMQGCVCTGVWVIAHECGHHAFSDYQLLDDIVGLVLHSSLMVPYFSWKYSHRRHHSNTGSLERDEVFVPKKKEAMPWYTPYVYNNPVGRLLHIVVQLTLGWPLYLTTNASGRPYPRFACHYDPYGPIYNDRERMQIFISDAGVMAVSFGLYKLAATFGFWWVVRIYAVPLLIVNAWLVLITYLQHTHPALPHYDSSEWDWLRGALATMDRDYGILNRVFHNITDTHVAHHLFSTMPHYHAMEATKAIKPILGEYYQFDPTPVAKATWREARECIYVEPEDRKGVFWYNNKF

SEQ ID NO: 10 - protein, 382 aa, synthetic construct
MTEKEREEQQKLLGRAGNGAAVQRSPTDKPPFTLGQIKKAIPPHCFQRSVIKSFSYVVHDLVIVAALLYFALVMIPVLPSGMEFAAWPLYWIAQGCVLTGVWVIAHECGHHAFSDYSVLDDIVGLVLHSSLLVPYFSWKYSHRRHHSNTGSLERDEVFVPKQKSAMAWYTPYVYHNPIGRLVHIFVQLTLGWPLYLAFNVSGRPYPRFACHFDPYGPIYNDRERVQIFISDVGVVSAGLALFKLSSAFGFWWVVRVYGVPLLIVNAWLVLITYLQHTHPALPHYDSSEWDWLRGALATVDRDYGILNKVFHNITDTHVAHHLFSTMPHYHAMEATKAIRPILGEYYQFDPTPVAKATWREAKECIYVEPEDNKGVFWYNNKF

SEQ ID NO: 11 - protein, 381 aa, synthetic construct
MTEKEREEQQKLLGRAGNGAAVQRSPTDKPPFTLGQIKKAIPPHCFQRSVIKSFSYVVHDLVIVAALLYFALVMIPVLPSGMEFAAWPLYWIAQGCVLTGVWVIAHECGHHAFSDYSVLDDIVGLVLHSSLLVPYFSWKYSHRRHHSNTGSLERDEVFVPKQKSAMAWYTPYVYHTIGRLVHIFVQLTLGWPLYLAFNVSGRPYPRFACHFDPYGPIYNDRERVQIFISDVGVVSAGLALFKLSSAFGFWWVVRVYGVPLLIVNAWLVLITYLQHTHPALPHYDSSEWDWLRGALATVDRDYGILNKVFHNITDTHVAHHLFSTMPHYHAMEATKAIRPILGEYYQFDPTPVAKATWREAKECIYVEPEDNKGVFWYNNKF

SEQ ID NO: 12 - protein, 390 aa, Oryza sativa
MGTSSRPTTVKEGKKLEAPRRAGSHAAVKRSPVDKPPFTLGDIRKAIPPHCFHRSVIKSFSYLLHDLAIAAGLLYFALVVIPALPGVLRLVAWPFYWAAQGCFLFGVWIIAHECGHHAFSGHALLDDTLGLVLHSWLLAPYFSWKYTHQRHHSNTSSQERDEVFVPRFKSDLPWYSPYVYKYNNPVARLLLLVVQLTVGWPMYLVFNTWGRQYPRFASHFDPSGPIYKGRERVFIAISDIGMLAVSLALYRLAEGYGFWWVVRVYGVPLLVVNAWLVVVTYLHHTHRAIPHYDSSEWDWLRGALATVDRDYSFLNRVFHNITDTHVVHHLFPTIPHYHAVEATKAIRPILGEYYQFDPTPIVKAIWREAKECIYIQSEDHKGVFWYSNKF

SEQ ID NO: 13 - protein, 362 aa, Oryza sativa
MQRSPVDKPPFTLGDIKKAIPPHCFHRSVIKSFSYLLHDLAIAAGLLYFALVGIPALPSILRLVAWPLYWAAQGSVLTGVWVIGHECGHHAFSDYLLLDNLVGLVLHSALLTPFFSWKYSHRRHHANTGSMEKDEVYVAKKKSALPWYTPYVFGNPVGRLVYIALQLTLAWPLYLAFNLSGQPYPRLVTCHYDPYSPLFSDQERVQVLVSDAAILAVLLALHRLTAAYGLWWVVRVYGVPVMIVGALFVLITYLHHTHRALPHYDSSEWEWLRGSLATVDRDYGVLNRVLHNVTDTHVLHHLFPSMPHYHAMEATRAARPVLGEYYKFDRTPIIEATWREAKECMYVEPRERDGIYWYNNKF

SEQ ID NO: 14 - protein, 223 aa, Oryza sativa
MAGGRRWGGWREQEPPRRAGSSAAVQRFHRSVIKSFSYLLRDVAIAAGLLNFALVGIPVLPAGVLRPPRRLAVLLGRAGLLPVRGVDHRARVRAPRAPRRHPRSGPALVASGTILLVEIQPPAAPLQHQLTGARRGVRPQVQVRSAVELPVRVQVQQRPVARLLLLGMQLTVGWPMYLVFNTWGRWYPRFASHFDPSGAIYMRRERVFIAISDIGMLAVSLAL

SEQ ID NO: 15 - DNA, 1167 bp, Oryza sativa
  source 1..1167: mol_type = other DNA
atgggtgccggcggcaggatgacggagaaggagcgggaggagcagcagaagctgctcggccgcgccggcaatggcgcggccgtgcagcggtcgccgacggacaagccgccgttcacgctggggcagatcaagaaggccatcccgcctcactgcttccagcgctcggtgatcaagtccttctcctacgtggtccatgacctcgtgatcgtcgccgcgctgctctacttcgcgctggtcatgatccccgtgctgccgagcgggatggagttcgcggcatggccgctctactggatcgcgcagggctgcgtgctcaccggcgtgtgggtcatcgcgcacgagtgcggccaccatgccttctccgactactcggtgctcgacgacatcgtcggcctcgtgctgcactcgtcgctgctcgtcccctacttctcgtggaagtacagccaccggcgccaccactccaacaccgggtcgctggagcgcgacgaggtgttcgtcccgaagcagaagtcggcgatggcgtggtacaccccgtacgtgtaccacaacccgatcggccggctggtgcacatcttcgtgcagctcaccctcgggtggccgctgtacctggcgttcaacgtgtccggccgcccgtacccgcgcttcgcgtgccacttcgacccctacggcccgatctacaacgaccgggagcgcgtccagatcttcatctccgacgtcggcgtcgtgtccgcggggctcgccctgttcaagctgtcgtcggcgttcgggttctggtgggtggtgcgcgtctacggcgtgccgctgctgatcgtgaacgcgtggctggtgctcatcacctacctgcagcacacccacccggcgctgccgcactacgactcgagcgagtgggactggctccgcggcgcgctggccaccgtggaccgcgactacggcatcctcaacaaggtgttccacaacatcacggacacgcacgtcgcgcaccacctcttctccaccatgccgcactaccacgccatggaggccactaaggcgatccgccccatcctcggcgagtactaccagttcgacccgacgcccgtcgccaaggcgacatggcgcgaggccaaggagtgcatctacgtcgagcctgaggacaacaagggcgtcttctggtacaacaacaagttctaa

SEQ ID NO: 16 - DNA, 1173 bp, Oryza sativa
  source 1..1173: mol_type = other DNA
atgggtaccagcagccggccgacgacggtgaaggaggggaagaaactagaggcgccccgccgtgccggcagccatgcagccgtgaagcgctctccggtggacaagccgccgttcacgctgggcgacatcagaaaggccatcccaccgcactgcttccaccgctccgtgatcaagtcattctcctacctgctccacgaccttgccattgccgcgggcctcctctactttgctctggtcgtcatccctgccctcccaggcgtcctccgcctcgtcgcctggccgttctactgggccgcgcaggggtgcttcctgttcggggtgtggatcatcgcgcacgagtgcgggcaccacgcgttctcgggccacgcactcctcgacgacaccctcggcctggtcctgcactcatggctcctagcgccatacttctcgtggaaatacacccaccaacggcaccactccaacaccagctcacaggagcgcgacgaggtgttcgtccccaggttcaagtccgacctgccgtggtactccccatacgtgtacaagtacaacaaccccgtcgctcggctgctgctcctcgtcgtgcagctcaccgtcgggtggccgatgtatttggtgttcaacacctggggtcgccagtacccaaggttcgccagccacttcgatccctctgggcccatctacaaggggcgggagcgcgtcttcatcgccatctcggacatcggcatgctggccgtgtcgctcgcgctgtacaggcttgcggagggttacgggttttggtgggtggtgcgcgtctacggcgtgccgctgcttgtcgtcaacgcgtggcttgtggtcgtcacgtacctgcatcacactcaccgggcgatcccacactacgactccagcgagtgggactggttgcgcggggcgctcgccaccgtggaccgcgactacagcttccttaaccgagtgtttcacaacatcacggacacacacgtcgtgcaccacctgttccctaccatcccgcactaccacgctgtggaggcgaccaaggcgatccgccctatcctcggcgagtactaccagttcgatcccacacccatcgtcaaggcgatatggcgcgaggctaaggagtgcatctacatccagtccgaggaccacaagggcgtcttctggtacagcaacaagttctag

SEQ ID NO: 17 - DNA, 1089 bp, Oryza sativa
  source 1..1089: mol_type = other DNA
atgcagcgctcaccggtggacaaaccgccgttcacgctgggggacataaagaaggccatcccgccgcactgcttccaccgctccgtgatcaagtcattctcctacctgctccacgaccttgccatcgccgctggcctcctctactttgctctggtcggcatccctgccctcccaagcatcctccgcctcgtcgcctggccgctctactgggccgcgcagggcagtgtactcaccggcgtgtgggtcatcgggcacgagtgtggccaccacgccttctcggactacttgctcctcgacaacctcgtgggcctagtgctccactcggcgcttctcacgcccttcttctcgtggaagtacagccaccggcggcaccacgccaacaccggctccatggagaaagacgaggtgtacgtcgcgaagaagaagtccgcgctgccgtggtacaccccgtacgtgttcggcaaccccgtcgggcggctggtgtacatcgccctgcagctcaccctcgcgtggccactctacctcgcgttcaacctgtccgggcagccgtacccacgcctcgtcacctgccactacgacccctacagcccgctgttcagcgaccaggagcgcgtccaagtcctcgtctccgacgccgccatcctggccgtgctgctcgcgctgcacaggctgacggcggcgtacgggctctggtgggtggtgcgcgtgtacggcgtgccggtgatgatcgtgggcgcgctgttcgtgctcatcacgtacctgcaccacacccaccgggcgctcccgcactacgactccagcgagtgggagtggctgcgtggctcgctcgccaccgtcgaccgcgactacggcgtcctcaaccgcgtgctgcacaacgtcacggacacgcacgtcctccaccacctcttccccagcatgccacactaccacgccatggaggccaccagggcagcgaggcccgtcctcggtgagtactacaagtttgaccgcacgcccatcatcgaggcaacatggcgtgaggccaaggagtgcatgtacgttgagcccagggagcgcgatggtatctactggtacaacaacaagttttag

SEQ ID NO: 18 - DNA, 672 bp, Oryza sativa
  source 1..672: mol_type = other DNA
atggcgggcggacggcggtggggcggatggcgtgaacaggagccgcctcgccgtgccggcagcagtgcagccgtgcagcgcttccaccgctccgtgatcaagtcattctcctacctgctccgtgacgtagccattgccgcgggcctcctcaactttgcgctggtcggcatccctgtcctccctgcaggcgtcctccggccgcctcgtcgcctggccgttctactgggccgcgcagggctgcttcctgttcgtggtgtggatcatcgcgcacgagtgcgggcaccacgcgctccaagacgacaccctcggtctggtcctgcacttgtggcttctggcaccatacttctcgtggaaatacagccaccagcggcaccactccaacaccagctcacaggagcgcgacgaggtgttcgtccccaggttcaagtccgatctgccgtggaactccccgtacgtgtacaagtacaacaacggcccgtcgcccggctactgctcctcggcatgcagctcactgtcgggtggccgatgtatttggtgttcaacacctggggtcgctggtacccgcggttcgccagccacttcgatccctccggagccatctacatgaggcgggagcgcgtcttcatcgccatctcggacatcggcatgctggccgtgtcgctcgcgctgtaa

SEQ ID NO: 19 - protein, 427 aa, Oryza sativa
MAGSLAASAFFPVPGSSPAASARSSKNTTGELPENLSVRGIVAKPNPSPGAMQVKAQAQALPKVNGTKVNLKTTSPDKEDIIPYTAPKTFYNQLPDWSMLLAAVTTIFLAAEKQWTLLDWKPKKPDMLADTFGFGRIIQDGLVFRQNFLIRSYEIGADRTASIETLMNHLQETALNHVKTAGLLGDGFGATPEMSKRNLIWVVSKIQLLVERYPSWGDMVQVDTWVAAAGKNGMRRDWHVRDYNSGQTILRATSVWVMMNKNTRRLSKMPDEVRAEIGPYFNGRSAISEEQGEKLPKPGTTFDGAATKQFTRKGLTPKWSDLDVNQHVNNVKYIGWILESAPISILEKHELASMTLDYRKECGRDSVLQSLTAVSGECDDGNTESSIQCDHLLQLESGADIVKAHTEWRPKRAQGEGNMGFFPAESA

SEQ ID NO: 20 - protein, 425 aa, Oryza sativa
MAGSLAASAFFPGPGSSPAASARSSKNAAVTGELPENLSVRGIVAKPNPPPAAMQVKAQAQTLPKVNGTKVNLKTVKPDMEETVPYSAPKTFYNQLPDWSMLLAAITTIFLAAEKQWTLLDWKPKKPDMLVDTFGFGRIIQDGMVFRQNFMIRSYEIGADRTASIETLMNHLQETALNHVRTAGLLGDGFGATPEMSKRNLIWVVSKIQLLVEQYPAWGDTVQVDTWVAAAGKNGMRRDWHVRDYNSGRTILRATSVWVMMHKKTRRLSKMPDEVRAEIGPYFNDRSAITEEQSEKLAKTGNKVGDDATEQFIRKGLTPRWGDLDVNQHVNNVKYIGWILESAPISVLEKHELASMTLDYRKECGRDSVLQSLTTVSGECTSIGADKQASAIQCDHLLQLESGADIVKAHTEWRPKRSHAAAENA

SEQ ID NO: 21 - protein, 298 aa, Oryza sativa
MAGSLAASAFFPGPGSSPAASARSSKNAAVTGELPENLSVCGIVAKPNPPPAAMQVKAQAQTLPKVNGTKVNLKTVKPDMEETVPHSAPKTFYNQLPDWSMLLAAITTIFLAAEKQWTLLDWKPKKPDMLVDTFGFGRIIQDGMVFRQNFMIRSYEIGADRTASIETLMNHLQETALNHVRTAGLLGDGFGATPEMSKRNLIWVVSKIQLLVEQYPAWGDMVQVDTWVAAAGKNGMRRDWHVRDYNSGRTILRATSVWVMMHKKTRRLSKMPDEVRAEIGPYFNDRSAITEEQSEKLA

SEQ ID NO: 22 - protein, 357 aa, Oryza sativa
MAGSIAASAFLPGSPAAAPPKSVLGERPDSLDVRGIAAKPGSSSSAAALRAGKTRTHAAIPKVNGGSSALADPEHDTMSSSSSSAAPRTFYNQLPDWSMLLAAITTIFLAAEKQWTLLDWKPKRPDMLTDTFGFGRMIHEGLMFRQNFSIRSYEIGADRTASIETLMNHLQETALNHVKSAGLLGDGFGSTPEMSKRDLFWVVSQMQAIVERYPCWGDTVEVDTWVGAHGKNGMRRDWHIRDSVTGHTILKATSKWVMMHKLTRRLARIPDEVRTEIEPYFFEHASIVDEDNQKLPKLPDIEGANVAKYVRTGLTPRWADLDINQHVNNVKYIGWILEVKKVPLLCSSLLPLLTPLA

SEQ ID NO: 23 - protein, 697 aa, Oryza sativa
MPAALKPYRDDELRNLRGDDQQGPYEEHDRVYRYDVYNDLGSPDSGNPRPILGGSPDTPYPRRGRTGRKPTTTDPDSESRLSLVEQIYVPRDERFGHLKMADFLGYSIKAIAEGIVPAIRTYVDTTPGEFDSFQDILDLYEGGLKLPDVPALEELRKRFPLQLVKDLLPAAGDYILKLPMPQIIKQDKEAWRTDEEFAREVLAGVNPMMITRLTEFPPKSSLDPSKFGDHTSMITAAHIGSNLEGLTVQQALDSNRLYILDHHDRFMPFLIDVNGLEGNFIYATRTLFFLRGDGTLAPLAIELSEPMIQGDVTAAKSTVYTPASTGVEAWVWQLAKAYVAVNDSGWHQLISHWLNTHAVMEPFVIATNRQLSVTHPVHKLLSPHYRDTMTINALARQTLINAGGIFEMTVFPGKYALWMSSMVYKNWNFTEQGLPADLIKRGVAVEDATSPYKVRLLIKDYPYAADGLEIWHAIEQWVGEYLAIYYTDDGVLRGDAELQAWWAEVREVGHGDLKGAAWWPRMDAVSELRDACTTIIWIASALHAAVNFGQYPYAGYLPNRPTVSRRRMPEPGTEAYGELGRDPERAFIRTITSQLQTIIGISLIEVLSKHSSDEVYLGQRDTPAWTSDARALEAFRRFSDRLVEIEGKVVGMNGDAGLKNRNGPAEFPYMLLYPNTSDVTGAAAGITAKGIPNSISI

SEQ ID NO: 24 - protein, 866 aa, Oryza Brachyantha
MLGGLIDSLTGSSKQSRLKGTVVLMRKNVLDLNDFGATVIDGLGEFVGKGVTCQLISSTAVDQNNGNRGKVGAEANLEQWPTSSLPSLTTGESRFGVTFDWEVEKLGVPGAVIVKNHHRNEFYLKTITLDSVPGRAGAVVFVANSWVYPAAKYRYDRVFFANDAYLPSQMPAALKPYRDDELRNLRGDDQQGPYQEHDRVYRYDVYNDLGEPDSGNPRPILGGSPDRPYPRRGRTGRKPTTKDADSESRLPLVQQIYVPRDERFGHLKMADFLGYSIRAIAEGVVPAIRTYVDTTPGEFDSFQDIVNLYEGCLRLPDVPALQELRKRFPLQLIKDLLPASGDYILKLPMPHIIKQDKQAWRTDEEFAREVLAGLNPMMITRLTEFPPKSTLDPSKFGDHTSTITAAHIENSLEGLTVQQALDSNRLYILDHHDRFMPFLIDVNNLEGNFIYATRTLFFLRGDGTLAPLAIELSEPLIQGGVTAAKSTVYTPASTGVEAWVWQLAKAYVAVNDSGWHQLISHWLNTHAVMEPFVIATNRQLSVTHPVHKLLSPHYRDTMTINALARQTLINAGGIFEMTVFPGKYALWMSSMVYKNWNFTEQALPNDLIKRGVAVEDATSPYKVRLLIEDYPYAVDGLEIWHAIEQWVGEYLAIYYPDDGVLRGDAELQAWWTEVREVGHGDLRDAAWWPRMQSVSELRDACATIIWTASALHAAVNFGQYPYAGYLPNRPTVSRRRMPEPGTEAYAELGRDPERAFIRTVTSQLQTIIGISLIEVLSKHSSDEVYLGQRDTPAWTSDPKALEAFRRFSSRLVEIEGRVVGKNGDPGLKNRNGPAKLPYMLLYPNTSDVSGAAAGITAKGIPNSISI

SEQ ID NO: 25 - protein, 866 aa, Glycine max
MLLGGIIDGLTGANKHARLKGTVVLMRKNVLDLNDLGATVIDGISEFLGKGVTCQLISSTLVDANNGNRGRVGAEANLEQWLTSLPSVTTGESKFGVTFDWEVAKLGVPGAVIVKNNHTSEFFLKTITLDDVPGRGTVTFVANSWVYPVGKYRYSRVFFSNDTYLPSQMPPALKPYRDDELRNLRGDDQQGPYQEHDRVYRYDVYNDLGEPDGGNPRPILGGSADHPYPRRCRTGRKPTKTDPNSESRLSSLLEKIYVPRDERFGHLKMSDFLGHAIKAIAQGIIPAVRTYVDTSPKEFDSFQDIINLYEGGIKLPKIQALEDMRKLFPLQLVKDLLPAGGDYLLKLPIPEIIKESKDAWRTDEEFAREVLAGVNPVVITRLTEFPPKSTLDPSKYGDHTSTITAEHIEKNLEGLTVQQALDGNRLYILDHHDRFMPFLIDVNNLEGNFIYATRALFFLRADGRLAPLAIELSEPYIDGGLTKAKSKVYTPASAGVEAWVWQLAKAYVAVNDSGWHQLISHWLNTHAVMEPFVIATNRQLSVTHPVHKLLHPHFRDTMTINALARQTLINGGGIFELTVFPGKYALAMSSVVYKDWNFTEQGLPADLVKRGVAVEDPSSPYKVRLLIQDYPYASDGLAIWHAIEQWVGEYLAIYYPDDAALRGDEELQAWWKEVREVGHGDLKDAAWWPKMQAVSELASACTTIIWIASALHAAVNFGQYPYAGYLPNRPTVSRRRMPEPGSKEYEELERYPERGFIHTITSQIQTIIGISLIEILSKHSSDEVYLGQRDTPEWTSDAKAKAAFKRFSDALVAIEGKVVGENRDPQLKNRNGPAEFPYMLLYPNTSDHSGAAAGLTAKGIPNSISI

SEQ ID NO: 26 - protein, 864 aa, Zea mays
MLSGIIDGLTGANKHARLKGTVVLMRKNVLDLNDFGATVVDSISEFLGKGVTCQLISSTLVDANNGNRGRVGAEANLEQWLTSLPSLTTGESKFGVTFDWEVEKLGVPGAVVVKNNHAAEFFLKTITLDDVPGRGAVTFVANSWVYPAGKYRYNRVFFSNDTYLPSQMPAALKPYRDDELRNLRGDDQQGPYQEHDRVYRYDVYNDLGEPDGGNPRPILGGSADHPYPRRCRTGRKPTKTDPNSESRLSLVEQIYVPRDERFGHLKMSDFLGYSIKAITQGIIPAVRTYVDTTPGEFDSFQDIINLYEGGIKLPKIQALEDMRKLFPLQLVKDLLPAGGDYLLKLPIPQIIQEDKNAWRTDEEFAREVLAGVNPMVITRLTEFPPKSTLDPSKYGDHTSTITAEHIEKNLEGLTVQQALDGNRLYILDHHDRFMPFLIDVNNLEGNFIYATRTLFFLRGDGRLAPLAIELSEPYIDGDLTVAKSKVYTPASSGVEAWVWQLAKAYVAVNDSGWHQLVSHWLNTHAVMEPFVIATNRQLSVTHPVHKLLSSHFRDTMTINALARQTLINGGGIFEMTVFPGKYALGMSSVVYKSWNFTEQGLPADLVKRGVAVADPSSPYKVRLLIEDYPYASDGLAIWHAIEQWVGEYLAIYYPDDGALRGDEELQAWWKEVREVGHGDHKDAPWWPKMQAVSELASACTTIIWIASALHAAVNFGQYPYAGYLPNRPTVSRRRMPEPGSKEYEELERDPERGFIHTITSQIQTIIGISLIEILSKHSSDEVYLGQRDTPEWTSDARALAAFKRFSDALVKIEGKVVGENRDPQLRNRNGPAEFPYMLLYPNTSDHSGAAAGLTAKGIPNSISI

SEQ ID NO: 27 - protein, 862 aa, Avena sativa
MLLGGLIDNLTGANKHARLKGTVVLMRKNVLDLNDFGATIIDGVSEFLGKGVTCQLISSTVVDNNNGNRGKVGAEAGLEQWLTSLPSLTTGESKFGLTFDWEVDKLGVPGAIVVNNYHSAQFFLKTITLDDVPGRAGKLTFVANSWIYPAEKYRYNRVFFANDTYLPSQMPAALKPYRDDELRNLRGDDQQGPYEEHDRVYRYDVYNDLGEDRPVLGGTADHPYPRRGRTGRKPNPNDPSLESRLSLLEQIYVPRDEKFGHLKMSDFLGYSIKAITQGILPAVRTYVDCTPGEFDSFQDIINLYEGGIKLPKIAALEELRKRFPFQLLKDLLPVGGDFLLKLPLPHIIKEDKQAWRTDEEFAREVLAGVNPVMITRLTEFPPKSTLDPSKYGDHTSTITAAHIEKNLEGLTVQQALEGNKLYILDHHDRFMPFLIDVNNLDGNFIYATRTLFFLRGDGRLTPLAIELSEPFIQDGLTTAKSKVYTPVPSGSVEGWVWELAKAYVAVGDSGWHQLVSHWLNTHAVMEPFVIATNRQLSVTHPVHKLLSPHYRDTMTINALARQTLINAGGIFEMTVFPGKFALGMSSVVYKDWNFAEQGLPDDLLRRGVAVPDPSSPYKVRLLIEDYPYAADGLAIWHAIEQYVTEYLAIYYPDDAVLQDDVELQAWWKEAREVGHGDLKDAPWWPSMQTVAELAKSCATIIWIASALHAAVNFGQYPYAGYLPNRPTVSRRRMPEPGTQEYAELERDPERAFIHTITSQIQTIIGISLLEVLSKHSSDELYLGQRDTPEWTSDPKALAVFQRFSDRLVDIESKVVGMNHDPQLKNRNGPAKLPYMLLYPNTSDRKGDAAGLTAKGIPNSISI

SEQ ID NO: 28 - protein, 862 aa, Hordeum vulgare
MLLGGLIDTLTGANKSARLKGTVVLMRKNVLDLNDFGATIIDGIGEFLGKGVTCQLISSTAIDQDNGGRGKVGAEAELEQWVTSLPSLTTGESKFGLTFDWEVEKLGVPGAIVVNNYHSSEFLLKTITLHDVPGRSGNLTFVANSWIYPAANYRYSRVFFANDTYLPSQMPAALKPYRDDELRNLRGDDQQGPYQEHDRIYRYDVYNDLGEGRPILGGNSDHPYPRRGRTGRKPNASDPSLESRLSLLEQIYVPRDEKFGHLKTSDFLGYSIKAITQGILPAVRTYVDTTPGEFDSFQDIINLYEGGIKLPKVAALEELRKQFPLQLIKDLLPVGGDSLLKLPVPHIIQENKQAWRTDEEFAREVLAGVNPVMITRLTEFPPKSSLDPSKFGDHTSTITAEHIEKNLEGLTVQQALESNRLYILDHHDRFMPFLIDVNNLPGNFIYATRTLFFLRGDGRLTPLAIELSEPIIQGGLTTAKSKVYTPVPSGSVEGWVWELAKAYVAVNDSGWHQLVSHWLNTHAVMEPFVISTNRHLSVTHPVHKLLSPHYRDTMTINALARQTLINAGGIFEMTVFPGKFALGMSAVVYKDWKFTEQGLPDDLIKRGMAVEDPSSPYKVRLLVSDYPYAADGLAIWHAIEQYVSEYLAIYYPNDGVLQGDTEVQAWWKETREVGHGDLKDAPWWPKMQSVPELAKACTTIIWIGSALHAAVNFGQYPYAGFLPNRPTVSRRRMPEPGTQEYVELERDPERAFIHTITSQIQTIIGVSLLEVLSKHSSDELYLGQRDTPEWTSDPKALEVFKRFSDRLVEIESKVVGMNHDPELKNRNGPPKFPYMLLYPNTSDHKGAAAGLTAKGIPNSISI

SEQ ID NO: 29 - protein, 861 aa, Triticum aestivum
MILGGLVDSLTGANKSARLKGTVVLMRKNVLDLNDFGATIMDGIGEFIGKGVTCQLISSTLVDHDNGGRGKVGAEAELEQWVTSLPSLTTGESKFGLTFDWEVEKLGVPGAIIVNNHHSSEFLLKTVTLHDVPGRGNLSFVANSWIYPVGSYTYSRVFFANDTYLPSQMPAALKPYRDDELRNLRGDDRQGPYQEHDRVYRYDVYNDLGEGRPVLGGSADHPYPRRGRTGRKPNANDPSLESRLSLLEQIYVPRDEKFGHLKTSDFLGYSIKAITQGILPAVRTYVDTTPGEFDSFQDIINLYEGGIKLPNVPALEELRKQFPLQLIKDLLPVGGDSLLKLPVPHIIQADKQAWRTDEEFSREVLAGVNPVMITRLTEFPPKSSLDPSKFGDHTSTVTAAHIEKNLEGLTVQQALESNRLYILDHHDRFMPFLIDVNNLPGNFIYATRTLFFLRGDGRLTPLAIELSEPVIQGGLTTAKSKVYTPVPSGSVEGWVWEFAKAYVAVNDSGWHQLVSHWLNTHAVMEPFVISTNRQLSVTHPVHKLLSPHYRDTMTINALARQTLINAGGIFEMTVFPGKFALGMSSVVYKDWKFTEQGLPDDLIKRGMAVEDPSSPYKVRLLVSDYPYAADGLAIWHAIEQYVSEYLAIYYPNDGVVQGDVELQAWWKEVREVGHGDLKVAPWWPRMQAVGELAKACTTIIWIGSALHAAVNFGQYPYAGFLPNRPTVSRRRMPEPGTEQYAELERDPERAFIHTITSQIQTIIGISLLEVLSKHSSDELYLGQRDTPEWTSDPKALEVFKRFSERLVEIESKVVGMNHDPQLLNRNGPAKFPYMLLYPNTSDHKGAAAGLTAKGIPNSISI

SEQ ID NO: 30 - RNA, 20 nt, synthetic construct
  source 1..20: mol_type = other RNA
gggtgccggcggcaggatga

SEQ ID NO: 31 - RNA, 20 nt, synthetic construct
  source 1..20: mol_type = other RNA
tacgtgtaccacaacccgat

SEQ ID NO: 32 - RNA, 20 nt, synthetic construct
  source 1..20: mol_type = other RNA
gacgagctccgcaacctgcg

SEQ ID NO: 33 - RNA, 20 nt, synthetic construct
  source 1..20: mol_type = other RNA
cgtgcgtgcagatccggact

SEQ ID NO: 34 - RNA, 20 nt, synthetic construct
  source 1..20: mol_type = other RNA
ctgaaccatgtgaaaactgc

SEQ ID NO: 35 - RNA, 20 nt, synthetic construct
  source 1..20: mol_type = other RNA
tcctggcagctgagaagcag

SEQ ID NO: 36 - RNA, 20 nt, synthetic construct
  source 1..20: mol_type = other RNA
gggctgctaggagatggttt

SEQ ID NO: 37 - RNA, 20 nt, synthetic construct
  source 1..20: mol_type = other RNA
atgattcggtcctacgagat

SEQ ID NO: 38 - protein, 6 aa, synthetic construct
NQHVNN

SEQ ID NO: 39 - protein, 412 aa, Arabidopsis thaliana
MVATSATSSFFPVPSSSLDPNGKGNKIGSTNLAGLNSAPNSGRMKVKPNAQAPPKINGKKVGLPGSVDIVRTDTETSSHPAPRTFINQLPDWSMLLAAITTIFLAAEKQWMMLDWKPRRSDMLVDPFGIGRIVQDGLVFRQNFSIRSYEIGADRSASIETVMNHLQETALNHVKTAGLLGDGFGSTPEMFKKNLIWVVTRMQVVVDKYPTWGDVVEVDTWVSQSGKNGMRRDWLVRDCNTGETLTRASSVWVMMNKLTRRLSKIPEEVRGEIEPYFVNSDPVLAEDSRKLTKIDDKTADYVRSGLTPRWSDLDVNQHVNNVKYIGWILESAPVGIMERQKLKSMTLEYRRECGRDSVLQSLTAVTGCDIGNLATAGDVECQHLLRLQDGAEVVRGRTEWSSKTPTTTWGTAP

SEQ ID NO: 40 - protein, 864 aa, synthetic construct
MLLGGLIDXLTGANKHARLKGTVVLMRKNVLDLNDFGATXIDGIXEFLGKGVTCQLISSTLVDXNNGNRGKVGAEANLEQWLTSLPSLTTGESKFGXTFDWEVEKLGVPGAXXVXNXHSSEFFLKTITLDDVPGRGXXTFVANSWXYPAGKYRYSRVFFANDTYLPSQMPAALKPYRDDELRNLRGDDQQGPYQEHDRVYRYDVYNDLGEPDGNPRPILGGSADHPYPRRGRTGRKPTXTDPSSESRLSLLEQIYVPRDERFGHLKMSDFLGYSIKAITQGILPAVRTYVDTTPGEFDSFQDIINLYEGGIKLPKVPALEELRKRFPLQLXKDLLPAGGDYLLKLPXPHIIKEDKQAWRTDEEFAREVLAGVNPVMITRLTEFPPKSTLDPSKFGDHTSTITAAHIEKNLEGLTVQQALDSNRLYILDHHDRFMPFLIDVNNLEGNFIYATRTLFFLRGDGRLAPLAIELSEPYIQGGLTTAKSKVYTPASSGVEAWVWQLAKAYVAVNDSGWHQLVSHWLNTHAVMEPFVIATNRQLSVTHPVHKLLSPHYRDTMTINALARQTLINAGGIFEMTVFPGKYALGMSSVVYKDWNFTEQGLPXDLIKRGVAVEDPSSPYKVRLLIEDYPYAADGLAIWHAIEQWVGEYLAIYYPDDGVLRGDXELQAWWKEVREVGHGDLKDAPWWPXMQAVSELAKACTTIIWIASALHAAVNFGQYPYAGYLPNRPTVSRRRMPEPGTEEYAELERDPERAFIHTITSQIQTIIGISLIEVLSKHSSDEVYLGQRDTPEWTSDPKALEAFKRFSDRLVEIEGKVVGMNHDPQLKNRNGPAKFPYMLLYPNTSDHXGAAAGLTAKGIPNSISI

SEQ ID NO: 41 - protein, 8 aa, synthetic construct
LNHVKTAG

SEQ ID NO: 42 - protein, 8 aa, synthetic construct
LNHVKTCW

SEQ ID NO: 43 - protein, 8 aa, synthetic construct
FLAAEKOW

SEQ ID NO: 44 - protein, 8 aa, synthetic construct
FLAAENSG

SEQ ID NO: 45 - protein, 8 aa, synthetic construct
FLAAEKTV

SEQ ID NO: 46 - protein, 10 aa, synthetic construct
MIRSYEIGAD

SEQ ID NO: 47 - protein, 9 aa, synthetic construct
MIRSYEDWC

SEQ ID NO: 48 - protein, 8 aa, synthetic construct
MIRSYDWR

SEQ ID NO: 49 - protein, 8 aa, synthetic construct
GLLGDGFG

SEQ ID NO: 50 - protein, 8 aa, synthetic construct
GLLGDFWL

SEQ ID NO: 51 - protein, 8 aa, synthetic construct
GLLGDGFW

SEQ ID NO: 52 - protein, 7 aa, synthetic construct
GLLGDFG

SEQ ID NO: 53 - protein, 8 aa, synthetic construct
GLLFWLNA

SEQ ID NO: 54 - protein, 6 aa, synthetic construct
MGAGGR

SEQ ID NO: 55 - protein, 9 aa, synthetic construct
PYVYHNPIG

SEQ ID NO: 56 - protein, 8 aa, synthetic construct
PYVYHTIG

(19) United States
(12) Patent Application Publication
(10) Pub. No.: US 2025/0304986 A1
(43) Pub. Date: Oct. 2, 2025

(54) MODIFIED CEREAL GRAIN

(71) Applicant: Commonwealth Scientific and Industrial Research Organisation, Acton (AU)

(72) Inventors: Jixun LUO, Acton (AU); Qing LIU, Acton (AU); Zhongyi LI, Acton (AU); Xue-Rong ZHOU, Acton (AU)

(73) Assignee: Commonwealth Scientific and Industrial Research Organisation, Acton (AU)

(21) Appl. No.: 18/707,501

(22) PCT Filed: Nov. 4, 2022
(86) PCT No.: PCT/AU2022/051328

§ 371 Date: May 3, 2024

(30) Foreign Application Priority Data
May 11, 2021 (AU) 2021903546

(51) Int. Cl. C12N 15/82; A23D 9/00; A23L 7/10; C12N 9/02

(52) U.S. Cl. CPC C12N 15/8247; A23D 9/00; A23L 7/115; C12N 9/0069; C12N 9/0071; C12Y 113/11012

(57) ABSTRACT

The present invention relates to cereal grain and bran, such as rice grain and bran, having a high oleic acid content and improved oil stability.

### CROSS-REFERENCE TO RELATED APPLICATIONS

This application is a § 371 national stage of PCT International Application No. PCT/AU2022/051328, filed Nov. 4, 2022, claiming priority of Australian Patent Application No. AU 2021903546, filed Nov. 5, 2021, the contents of each of which are hereby incorporated by reference into the subject application.

This application incorporates-by-reference nucleotide sequences which are present in the file named “241218_92410_SequenceListing_DH.xml”, which is 71 kilobytes in size, and which was created on Dec. 18, 2024 in the IBM-PC machine format, having an operating system compatibility with MS-Windows, which is contained in the xml file filed Dec. 18, 2024 as part of this application.

### FIELD OF THE INVENTION

The present invention relates to cereal grain and bran, such as rice grain and bran, having a high oleic acid content and improved oil stability.

### BACKGROUND OF THE INVENTION

Rice (Oryza sativa L.) is one of the most important staple foods for over half of the world population, especially in Asia which produces about 90% of the world total. The vast majority of rice in the world is eaten as “white rice” which is essentially the endosperm of the rice grain, having been produced by milling of harvested grain to remove the outer bran layer and germ (embryo and scutellum). This is done primarily because “brown rice” does not keep well on storage, particularly under hot tropical conditions. The nutritional quality and potential health benefits of brown rice have attracted increasing interest from nutritionists, breeders, and plant biotechnologists.

### Rice Bran

Rice bran is the outer brown coloured layer of the rice grain and it includes the embryo, pericarp, aleurone and sub-aleurone layer. Although it is known to be rich in minerals, proteins, oils and crude fiber rice bran is primarily obtained as a by-product of white rice milling. Current global production is approximately 66-75 million tons. In general rice bran is composed of 14-16% protein, 12-23% lipid and 8-10% crude fibre (Juliano, 1985).

### Rice Bran Oil

Rice bran is the source of rice bran oil (RBO). Interest in RBO as an edible oil, e.g. for cooking has been increasing as health benefits for humans has been demonstrated. Studies have shown that RBO consumption significantly decreases low-density lipoprotein cholesterol (LDL-C) and increases antioxidant capacity in hyperlipidemic subjects (Bumrungpert et al 2019; Berger et al., 2005). RBO health characteristics have been associated with the 3-4.5% high unsaponifiable important minor components such as tocopherols, phytosterols, terpenes and mixed isoprenoids compared to approximately 1% oil content of other vegetable oils. RBO generally contains 1.8% phytosterols, 1.2-1.7% gamma-oryzanol, tocotrienols up to 0.17% and tocopherols 0.08% (Pal and Pratap, 2017). These minor constituents are of increasing interest because some have been shown to exert beneficial effects on skin health, aging, eyesight and blood cholesterol or preventing breast cancer or cardiovascular disease (Theriault et al., 1999; Moghadasian and Frohlich, 1999). These bioactive components have also been shown to improve lipid profiles in rats fed a high cholesterol diet (Ha et al., 2005). Another important component found primarily in the bran is vitamin A precursors. However, these nutritional and health benefits are lost through the polishing of rice and the consumption of white rice.

### Modification of Fatty Acid Biosynthetic Enzymes in Cereals

In contrast to the considerable work done on fatty acid biosynthesis and modification in oilseeds, oil modification in cereals is relatively unexplored. This is probably due to the much lower levels of oils (about 1.5-6% by weight) in cereal grains and consequently the perceived lower importance of oils from cereals in the human diet.

TABLE 1
Fatty acid composition (wt % of total fatty acids) for
selected fatty acids of plant lipids associated with

| Plant | 16:0 | 18:1 | 18:2 |
|---|---|---|---|
| Wheat | 35-44 | 6-14 | 42-52 |
| Barley | 55 | 4 | 36 |
| Rye | 23 | 41 | 35 |
| Oat | 40 | 22 | 35 |
| Maize | 37 | 11 | 46 |
| Maize- High amylose | 36 | 20 | 38 |
| Maize- waxy | 36 | 23 | 36 |
| Millet | 36 | 28 | 29 |
| Rice | 37-48 | 9-18 | 29-46 |

Data adapted from: Morrison (1988).

The nutrient-rich outer rice bran layer obtained through polishing the outer layers of the rice grain is an excellent food source, containing antioxidant compounds such as tocotrienols and gamma-oryzanol which is also a phytoestrogen (Rukmini and Raghuram, 1991). The bioactive compounds present in rice bran oil have been found to lower cholesterol in humans (Most et al., 2005).

There is a need to improve the cereal grain lipid profile in the bran layer to enhance the utility and shelf-life of wholegrain cereal and prevent rancidity of bran and RBO without the need for further processing. There is also a need for cereal varieties, such as rice, that produce grain with an improved oil composition for health benefits, which at the same time is more stable on storage, allowing greater use of, for example, brown rice, rice bran and RBO in the human diet.

### SUMMARY OF THE INVENTION

The present inventors have produce cereal grain and bran with improved oil characteristics.

Thus, in a first aspect the present invention provides fertile cereal grain comprising a genetically modified FAD2-1 gene and a genetically modified LOX3 gene, wherein the grain comprises
i) at least some FAD 2-1 protein activity, wherein the FAD2-1 activity is reduced when compared to a wild type cereal grain, and
ii) reduced LOX3 protein activity when compared to the wild type cereal grain.

In an embodiment, the cereal grain is rice, sorghum, wheat, oats, rye, barley or maize grain. In an embodiment, the grain is a sorghum grain. In an embodiment, the grain is a rice grain.

In an embodiment, oil extracted from the grain is more stable than oil extracted from the wild type cereal therefrom.

In an embodiment, the grain has a total fatty acid content comprising at least 50%, at least 60%, at least 70%, at least 75%, between 50% and 80%, between 55% and 75%, between 55% and 70%, oleic acid (w/w dry weight). In an embodiment, wherein the grain has a total fatty acid content comprises between 55% and 75% oleic acid (w/w dry weight). In an embodiment, the grain has a total fatty acid content which comprises between 55% and 65% oleic acid (w/w dry weight).

In an embodiment, the grain has a total fatty acid content comprising less than 22%, less than 21%, less than 20%, less than 18%, less than 15%, between 15% and 22% or between 15% and 21%, palmitic acid (w/w dry weight). In an embodiment, the grain has a total fatty acid content comprising between 10% and 15% palmitic acid (w/w dry weight). In an embodiment, the grain has a total fatty acid content comprising between 10% and 13% palmitic acid (w/w dry weight).

In an embodiment, the grain has a total fatty acid content comprising less than 20%, less than 15%, less than 10%, less than 5%, between 2% and 20% or between 5% and 15%, linoleic acid (w/w dry weight). In an embodiment, the grain has a total fatty acid content comprising between 15% and 25% linoleic acid (w/w dry weight).

In an embodiment, the grain has a total fatty acid content comprising between 55% and 65% oleic acid, between 10% and 15% palmitic acid and between 15% and 25% linoleic acid.

In an embodiment, the grain is homozygous for a FAD 2-1 allele which produces a reduced amount of FAD2-1 protein and/or which encodes a FAD2-1 protein with reduced FAD2-1 protein activity, a LOX3 knockout, a FATB2 knockout, a FATB3 knockout, and a FATB4 knockout.

In an embodiment, the grain is homozygous for a FAD 2-1 allele which produces a reduced amount of FAD2-1 protein and/or which encodes a FAD2-1 protein with reduced FAD2-1 protein activity, a LOX3 knockout, a FATB1 knockout and a FATB4 knockout.

In an embodiment, the grain is homozygous for a FAD 2-1 allele which produces a reduced amount of FAD2-1 protein and/or which encodes a FAD2-1 protein with reduced FAD2-1 protein activity, a LOX3 knockout, a FATB1 knockout, a FATB2 knockout, a FATB3 knockout, and a FATB4 knockout.

In an embodiment, the grain has no LOX3 protein activity. For example, the genetic modification is a premature stop codon in the LOX3 gene.

In an embodiment, the grain is homozygous for the genetic modification in the LOX3 gene. In an embodiment, the genetic modification of the LOX3 gene is a premature stop codon in the LOX3 gene.

In an embodiment, the grain is homozygous for the genetic modification in the FAD2-1 gene.

In an embodiment, the grain is heterozygous for the genetic modification in the FAD2-1 gene.

In an embodiment, the grain comprises a wild type FAD2-1 allele and a knock out FAD 2-1 allele.

In an embodiment, the grain comprises a wild type FAD2-1 allele and a FAD 2-1 allele which produces a reduced amount of FAD2-1 protein and/or which encodes a FAD2-1 protein with reduced FAD2-1 protein activity.

In an embodiment, the grain comprises a FAD 2-1 allele which produces a reduced amount of FAD2-1 protein and/or which encodes a FAD2-1 protein with reduced FAD2-1 protein activity and a knock out FAD 2-1 allele.

In an embodiment, the genetically modified FAD2-1 gene encodes a mutant FAD2-1 protein. In an embodiment, the mutant FAD2-1 has between 5% and 95% less, between 20% and 80% less, between 40% and 70% less, or between 50% and 60% less, Δ12 desaturase activity than a wild type FAD2-1 protein. In an embodiment, the grain the mutant FAD2-1 has between 5% and 95% less, between 20% and 80% less, between 40% and 70% less, or between 50% and 60% less, Δ12 desaturase activity than a wild type FAD2-1 protein such as a FAD 2-1 protein consisting an amino acid sequence set forth in any one of SEQ ID NO's 1 to 9.

In an embodiment, the FAD2-1 protein with reduced FAD2-1 protein activity comprises or consists of an amino acid sequence as set forth in SEQ ID NO: 10 or SEQ ID NO:11. In an embodiment, the FAD2-1 protein with reduced FAD2-1 protein activity has a modified translation start site.

In an embodiment, the grain has wild type activity for other FAD2 genes in the genome of the grain. For example, rice grain of the invention has wild type FAD 2-2, FAD 2-3 and FAD 2-4 activity.

In an embodiment, one or both of the genetic modifications were introduced by gene editing an ancestral cereal plant.

In an embodiment, the grain has reduced FATB activity when compared to the wild type cereal grain. In an embodiment, the FATB is FATB1.

In an embodiment, the grain does not comprise exogenous dsRNA.

In a further aspect, the present invention provides cereal bran comprising genetically modified cells comprising
i) at least some FAD 2-1 protein activity, wherein the FAD2-1 activity is reduced when compared to a wild type cereal bran, and
ii) reduced LOX3 protein activity when compared to the wild type cereal bran.

The bran may have any of the relevant features defined above for the cereal grain of the invention such as the fatty acid profile. For example, in an embodiment, the bran is rice bran.

In an aspect, the present invention provides extracted cereal grain oil, or cereal bran oil, having a total fatty acid content comprising between 50% and 80%, or between 55% and 80%, oleic acid (w/w dry weight), and having an induction time of at least 25 hours as measured by Rancimat test conducted at 110° C. at an airflow rate of 20 L/hr.

In another aspect, the present invention provides extracted cereal grain oil, or cereal bran oil, which is more stable than cereal oil extracted from a cereal grain or bran lacking i) and ii) of the invention. In an embodiment, extracted cereal grain or bran oil of this aspect has a total fatty acid content comprising between 50% and 80%, or between 55% and 80%, oleic acid (w/w dry weight).

In an embodiment, the cereal oil is rice, sorghum, wheat, oats, rye, barley or maize oil. In an embodiment, the bran oil is rice, sorghum, wheat, oats, rye, barley or maize bran oil. In an embodiment, the oil is a sorghum grain oil or bran oil. In an embodiment, the oil is a rice grain oil or bran oil.

In an embodiment, extracted cereal grain or bran oil of the invention has a total fatty acid content comprising between 55% and 75%, or between 55% and 70%, oleic acid (w/w dry weight). In an embodiment, extracted cereal grain or bran oil of the invention has a total fatty acid content comprises between 55% and 65% oleic acid (w/w dry weight).

In an embodiment, extracted cereal grain or bran oil of the invention has a total fatty acid content comprising less than 22%, less than 21%, less than 20%, less than 18%, less than 15%, between 15% and 22% or between 15% and 21%, palmitic acid (w/w dry weight). In an embodiment, extracted cereal grain or bran oil of the invention has a total fatty acid content comprising between 10% and 15% palmitic acid (w/w dry weight). In an embodiment, extracted cereal grain or bran oil of the invention has a total fatty acid content comprising between 10% and 13% palmitic acid (w/w dry weight).

In an embodiment, extracted cereal grain or bran oil of the invention has a total fatty acid content comprising less than 20%, less than 15%, less than 10%, less than 5%, between 2% and 20% or between 5% and 15%, linoleic acid (w/w dry weight). In an embodiment, extracted cereal grain or bran oil of the invention has a total fatty acid content comprising between 15% and 25% linoleic acid (w/w dry weight).

In an embodiment, extracted cereal grain or bran oil of the invention has a total fatty acid content comprising between 55% and 65% oleic acid, between 10% and 15% palmitic acid and between 15% and 25% linoleic acid.

In a further aspect, the present invention provides a substantially purified and/or recombinant mutant FAD 2-1 protein which has between 5% and 95% less, between 20% and 80% less, between 40% and 70% less, or between 50% and 60% less, Δ12 desaturase activity than a FAD2-1 protein consisting of the amino acid sequence set forth in SEQ ID NO: 1, than a corresponding wild type FAD2-1 protein.

Thus, this aspect excludes wild type FAD 2-1 proteins such as those consisting of an amino acid sequence set forth as any one of SEQ ID NO's 1 to 9.

In an embodiment, the mutant FAD 2-1 comprises an amino acid sequence which is at least 95%, at least 96%, at least 97%, at least 98%, at least 99%, or at least 95.5%, identical to the amino acid sequence set forth in and one or more of SEQ ID NOs 1 to 9.

In an embodiment, the mutant FAD 2-1 comprises an amino acid sequence which is at least 95%, at least 96%, at least 97%, at least 98%, at least 99%, or at least 95.5%, identical to the amino acid sequence set forth in SEQ ID NO:1.

In an embodiment, the mutant FAD 2-1 comprises an amino acid sequence which is at least 95%, at least 96%, at least 97%, at least 98%, at least 99%, or at least 95.5%, identical to the amino acid sequence set forth in SEQ ID NO:6.

In an embodiment, the protein comprises a sequence of amino acids set forth in SEQ ID NO:10 or SEQ ID NO:11.

In an embodiment, the mutant is an N-terminal truncation of the wild type protein. In an embodiment, the mutant lacks one or more or all of the first six amino acids of the wild type FAD 2-1 protein. In an embodiment, the mutant is encoded by a FAD2-1 gene with a genetically modified translation start site.

In another aspect, the present invention provides an isolated and/or exogenous polynucleotide encoding the protein of the invention.

In another aspect, the present invention provides a vector comprising the polynucleotide of the invention.

In an embodiment, the polynucleotide is operably linked to a promoter.

Also provided is a cell, preferably a rice cell, which comprises the genetic modifications as defined herein, the polynucleotide of the invention or the vector of the invention.

In an embodiment, the cell is a cereal plant cell. Examples of cereal plant cells of the invention include, but are not limited to, wheat, oats, rye, barley, rice, corn, sorghum or maize cells. In an embodiment, the cell is a sorghum cell. In an embodiment, the cell is a rice cell.

In a preferred embodiment, the cell is a rice grain cell such as a rice bran cell.

In an embodiment, the polynucleotide is integrated into the genome of the cell.

In a further aspect, the present invention provides a cereal plant comprising one or more or all of cereal grain of the invention, cereal bran of the invention, the protein of the invention, the polynucleotide of the invention, the vector of the invention or the cell of the invention. In an embodiment, the plant is a sorghum plant.

Also provided is a population of at least 100 plants, such as rice plants, of the invention growing in a field.

In yet another aspect, the present invention provides a method of producing the cell of the invention, the method comprising a step of introducing genetic modifications as defined herein, the polynucleotide of the invention or the vector of the invention, into a cell.

In another aspect, the present invention provides a method of identifying a FAD 2-1 protein with reduced FAD 2-1 protein activity, the method comprising
i) obtaining a polypeptide having an amino acid sequence which is at least 90%, least 95%, at least 96%, at least 97%, at least 98%, at least 99%, or at least 99.5% identical, but not identical, to the amino acid sequence set forth in any one or more of SEQ ID NO's 1 to 9,
ii) assessing FAD 2-1 protein activity of the polypeptide by determining the ability of the polypeptide to introduce a double bond into oleic acid at Δ12 position, and
iii) selecting a polypeptide which has some FAD 2-1 protein activity, but less FAD 2-1 protein activity than a protein consisting of an amino acid sequence set forth in any one of SEQ ID NO's 1 to 9.

In an embodiment, the polypeptide of part i) is assessed in comparison to a corresponding wild type FAD2-1. For example, in an embodiment the method comprises
i) obtaining a polypeptide having an amino acid sequence which is at least 90%, least 95%, at least 96%, at least 97%, at least 98%, at least 99%, or at least 99.5% identical, but not identical, to the amino acid sequence set forth in SEQ ID NO: 1,
ii) assessing FAD 2-1 protein activity of the polypeptide by determining the ability of the polypeptide to introduce a double bond into oleic acid at Δ12 position, and
iii) selecting a polypeptide which has some FAD 2-1 protein activity, but less FAD 2-1 protein activity than a protein consisting of an amino acid sequence set forth in SEQ ID NO:1.

In an embodiment, the polypeptide of i) is a N-terminal and/or C-terminal truncation of a wild type FAD2-1 polypeptide.

In another aspect, the present invention provides a method of producing a genetically modified cereal plant, the method comprising
i) introducing a genetic modification into a cereal cell such that it encodes a protein of the invention, and
ii) producing a plant from the cell.

In an embodiment, the method further comprises analysing the fertility of the plant, and selecting a plant which is fertile.

In an embodiment, the method further comprises analysing the fatty acid composition of grain and/or bran of the plant, or a descendent thereof, and selecting a plant which produces grain and/or bran having a total fatty acid content as defined herein.

In an embodiment, the cell does not encode a functional LOX3 protein.

In an embodiment, the method further comprising introducing a genetic modification such that the plant, or a descendent thereof, does not encode a functional LOX3 protein in its grain and/or bran.

In an embodiment, the method further comprises harvesting grain from the plant of step ii), the grain having the genetic modification(s).

In an embodiment, the method further comprises producing one or more generations of genetically modified progeny plants from the genetically modified grain, the progeny plants having the genetic modification(s).

In another aspect, the present invention provides a method of producing a cereal plant of the invention, the method comprising crossing a first genetically modified parental plant having grain comprising at least some FAD 2-1 protein activity, wherein the FAD 2-1 protein activity is reduced when compared to a wild type cereal grain, with a second genetically modified parental plant having grain comprising reduced LOX3 protein activity when compared to the wild type cereal grain.

In another aspect, the present invention provides a method of selecting a cereal plant of the invention, or grain from the plant, the method comprising the steps of
i) screening a population of cereal plants, grain or bran each of which were obtained from a mutagenic treatment of progenitor cereal cells, grain or plants, for the production of grain or bran as defined herein, or for the presence of the genetic modifications, and
ii) selecting from the population of step (i) a cereal plant or grain which produces grain as defined herein, thereby selecting the cereal plant or grain.

In an embodiment, step ii) comprises:
i) analysing a sample comprising DNA from a progeny plant, or grain therefrom, for the genetic modifications, and/or
ii) analysing the fatty acid content of the grain or bran therefrom.

In another aspect, the present invention provides a method for identifying a cereal plant of the invention, the method comprising the steps of
i) obtaining a nucleic acid sample from a cereal plant, and
ii) screening the sample for the presence or absence of a first genetic modification which reduces but does not abolish FAD 2-1 protein activity in grain of a plant when compared to a wild type cereal grain, and a second genetic modification which reduces LOX3 protein activity in grain of the plant when compared to a wild type cereal grain.

In another aspect, the present invention provides a process of producing extracted cereal grain and/or cereal bran oil, the process comprising;
i) obtaining grain and/or bran from a cereal plant of the invention, and
ii) extracting oil from the grain and/or cereal bran.

In an embodiment, the extracted oil is as defined herein.

In another aspect, the present invention provides a method of producing a cereal plant part, the method comprising,
a) growing a cereal plant, or at least 100 such cereal plants in a field, of the invention, and
b) harvesting the cereal plant part from the cereal plant or cereal plants.

In an embodiment, the part is grain.

In another aspect, the present invention provides a method of producing cereal flour, bran, wholemeal, malt, starch or oil obtained from grain, the method comprising;
a) obtaining grain of a plant of the invention, or the grain and/or bran of the invention, and
b) processing the grain to produce the flour, bran, wholemeal, malt starch or oil.

In an embodiment, the oil is cereal bran oil such as rice bran oil.

In another aspect, the present invention provides lipid or oil obtained, or obtainable, by the process of the invention.

In another aspect, the present invention provides a product produced from a plant of the invention, or from the grain and/or bran of the invention.

In an embodiment, the product comprises the genetic modifications.

In an embodiment, the product is a food ingredient, beverage ingredient, food product or beverage product.

In an embodiment, the food ingredient or beverage ingredient is selected from the group consisting of wholemeal, flour, bran, starch, malt and oil.

In an embodiment, the food product is selected from the group consisting of animal fodder, breakfast cereals, and snack foods.

In an embodiment, the beverage product is a packaged beverage or a beverage comprising ethanol.

In another aspect, the present invention provides a method of preparing a food or beverage ingredient of the invention, the method comprising processing grain of a cereal plant of the invention, the grain and/or bran of the invention, or bran, flour, wholemeal, malt, starch or oil from the grain, to produce the food or beverage ingredient.

In another aspect, the present invention provides a method of preparing a food or beverage product of the invention, the method comprising processing grain of a cereal plant of the invention, the grain and/or bran of the invention, or bran, flour, wholemeal, malt, starch or oil from the grain, to produce the food or beverage.

In another aspect, the present invention provides a method of preparing food, the method comprising cooking an edible substance in cereal oil, such as rice oil, of the invention.

Also provided is the use of a cereal plant of the invention or part thereof, or the grain and/or bran of the invention, as animal feed or food, or to produce feed for animal consumption or food for human consumption.

In another aspect, the present invention provides a composition comprising one or more of a polypeptide of the invention, a polynucleotide of the invention, a vector of the invention, a cell of the invention, or oil of the invention, and one or more acceptable carriers.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. cDNA sequence alignment of OsFAD2s from rice. The guide RNA target sequence for gRNA1 and gRNA2 in the FAD2 genes is shown by the Target 1 and Target 3 bar.

FIG. 2. CRISPR gene editing vector V1 in pYLCRISPR_Cas9Pubi-H.

FIG. 3. Alignment of translated protein sequences of OsFATB genes with Arabidopsis FATB1. The asterisk * shows the site of the catalytic triad (aspartate N-227, histidine H-229 and cysteine C-264) in the aligned sequences.

FIG. 4. Vector 2 FATB gRNA ligation product (Golden Gate, BsaI): pYLCRISPR_Cas9Pubi-H-V2.

FIG. 5. Half seed fatty acid composition. A, T₃ seeds of V1-13 and Neg; B, T₂ seeds from a single panicle of V1-13.

FIG. 6. A) Line genotype key is as follows: KD refers to the fad2-1 KD/KD+lox3KO genotype; LOX is FAD2WT+lox3-KO genotype; and Neg is the Negative control. B) FAD2-KO is the homozygous fad2-1 KO/KO line; Neg refers to the Negative Control; FAD2-KD refers to the fad2-1 KD/KD+lox3KO genotype; FAD2-KD/KO refers to the fad2-1 KD/KO+lox3-KO; and LOX3 refers to the FAD2-1WT+lox3 KO genotype.

FIG. 7. Half seed fatty acid composition T₃ seeds of V2 mutants as described in Tables 5 and 7. *For ease of reference the b1, b2, b3 and b4 refers to the presence of a mutated version of one or more of the FATB1, FATB2, FATB3 and FATB4 respectively. NEG is a Negative Control and Nip refers to wild type Niponnbare.

FIG. 8. Total fatty acid composition of high oleic and low palmitic acid genotypes. There are five major fatty acids in brown rice (16:0, 18:0, 18:1, 18:2, and 18:3) and some minor fatty acids, such as myristic (14:0) and 20:0.

FIG. 9. Oxidative stability of rice bran oil extract by Rancimat test. A, Total fatty acid composition of rice bran oil extract from genetically modified mutants and FAD2-RNAi line. B, KD refers to the fad2-1 KD/KD+lox3KO genotype; LOX is FAD2WT+lox3-KO genotype; and Neg is the Negative control. C, FAD2 is FAD2-RNAi silenced line, NEG is negative control.

FIG. 10. The production of hexanal compound from the rice bran samples of the gene edited mutants and FAD2 RNAi silenced lines in a 3 day storage stimulation assay. DO (Day 0) and D3 (Day 3) indicate the time point when samples were taken before and after the storage stimulation. A, KD refers to the fad2-1 KD/KD+lox3KO genotype; KK refers to the fad2-1 KD/KO+lox3KO genotype; LOX is FAD2WT+lox3-KO genotype; and Neg is the Negative control. B, FAD2 refers to the FAD2-RNAi silenced line and NC is the corresponding negative control.

FIG. 11. Alignment of wild type cereal FAD2-1 proteins.

FIG. 12. Alignment of wild type cereal LOX3 proteins.

### KEY TO THE SEQUENCE LISTING

SEQ ID NO: 1—Rice FAD 2-1
SEQ ID NO: 2—Barley FAD 2-1
SEQ ID NO: 3—Maize FAD 2-1
SEQ ID NO: 4—Brachypodium distachyon FAD 2-1
SEQ ID NO: 5—Brassica napus FAD 2-1
SEQ ID NO: 6—Glycine max FAD 2-1
SEQ ID NO: 7—Carthamus tinctorius FAD 2-1
SEQ ID NO: 8—Olea europaea FAD 2-1
SEQ ID NO: 9—Setaria italica FAD 2-1
SEQ ID NO: 10—Mutant FAD2-1A
SEQ ID NO: 11—Mutant FAD2-1B
SEQ ID NO: 12—Rice FAD 2-2
SEQ ID NO: 13—Rice FAD 2-3
SEQ ID NO: 14—Rice FAD 2-4
SEQ ID NO: 15—Polynucleotide sequence encoding rice FAD 2-1
SEQ ID NO: 16—Polynucleotide sequence encoding rice FAD 2-2
SEQ ID NO: 17—Polynucleotide sequence encoding rice FAD 2-3
SEQ ID NO: 18—Polynucleotide sequence encoding rice FAD 2-4
SEQ ID NO: 19—Rice FATB-1
SEQ ID NO: 20—Rice FATB-2
SEQ ID NO: 21—Rice FATB-3
SEQ ID NO: 22—Rice FATB-4
SEQ ID NO: 23—Rice LOX3
SEQ ID NO: 24—Oryza brachyantha LOX3
SEQ ID NO: 25—Glycine max LOX3
SEQ ID NO: 26—Maize LOX3
SEQ ID NO: 27—Avena sativa LOX3
SEQ ID NO: 28—Barley LOX3
SEQ ID NO: 29—Triticum aestivum LOX3
SEQ ID NO's 30 to 37—RNA guides
SEQ ID NO 38—FATB motif
SEQ ID NO 39—Arabidopsis FATB1
SEQ ID NO: 40—Cereal LOX3 consensus sequence
SEQ ID NO's 41 to 56—Amino acid motifs

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, genetic modification including gene editing, protein chemistry, food preparation and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−10%, more preferably +/−5%, more preferably +/1%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “genetically modified”, “genetic modification” or variants thereof refers to any genetic manipulation by man and includes introducing genes into cells by transformation or transduction, gene editing, mutating genes in cells and altering or modulating the regulation of a gene in a cell or organisms to which these acts have been done or their progeny and so on.

As used herein, an “oil” is a composition comprising predominantly lipid and which is a liquid at room temperature. For instance, oil of the invention preferably comprises at least 75%, at least 80%, at least 85% or at least 90% lipid by weight. Typically, a purified oil comprises at least 90% triacylglycerols (TAG) by weight of the lipid in the oil. Minor components of an oil such as diacylglycerols (DAG), free fatty acids (FFA), phospholipid and sterols may be present as described herein. In an embodiment, oil of the invention is grain and/or bran oil.

As used herein, the term “rice oil” refers to a composition obtained from the grain/seed, or a portion thereof such as the bran layer, of a rice plant which comprises at least 60% (w/w) lipid. Rice oil is typically a liquid at room temperature. The lipid comprises fatty acids that are at least 6 carbons in length. The fatty acids are typically in an esterified form, such as for example as triacylglycerols, phospholipid. Rice oil of the invention comprises oleic acid. Rice oil of the invention may also comprise at least some other fatty acids such as palmitic acid, linoleic acid, myristic acid, stearic acid and/or linolenic acid. The fatty acids may be free fatty acids and/or be found as triacylglycerols (TAGs). In an embodiment, at least 50%, more preferably at least 70%, more preferably at least 80% of the fatty acids in rice oil of the invention be found as TAGs. Rice oil of the invention can form part of the rice grain/seed or portion thereof such as the aleurone layer or embryo/scutellum, which together are referred to as “rice bran”. Alternatively, rice oil of the invention has been extracted from rice grain/seed or rice bran. An example of such an extraction procedure is provided in Example 1. Thus, in an embodiment, “rice oil” of the invention is “substantially purified” or “purified” rice oil that has been separated from one or more other lipids, nucleic acids, polypeptides, or other contaminating molecules with which it is associated in its native state. It is preferred that the substantially purified rice oil is at least 60% free, more preferably at least 75% free, and more preferably at least 90% free from other components with which it is naturally associated. In a preferred embodiment, upon extraction the ratio of oleic acid to linoleic acid, palmitic acid to oleic acid and/or palmitic acid to linoleic acid has not been significantly altered (for example, no greater than a 5% alteration) when compared to the ratio in the intact seed/grain or bran. In a further embodiment, the rice oil has not been exposed to a procedure, such as hydrogenation, which may alter the ratio of oleic acid to linoleic acid, palmitic acid to oleic acid and/or palmitic acid to linoleic acid when compared to the ratio in the intact seed/grain or bran. Rice oil of the invention may further comprise non-fatty acid molecules such as, but not limited to, γ-oryzanols and sterols.

Rice oil may be extracted from rice grain or bran by any method known in the art. This typically involves extraction with nonpolar solvents such as diethyl ether, petroleum ether, chloroform/methanol or butanol mixtures. Lipids associated with the starch in the grain may be extracted with water-saturated butanol. The rice oil may be “de-gummed” by methods known in the art to remove polysaccharides or treated in other ways to remove contaminants or improve purity, stability or colour. The triacylglycerols and other esters in the oil may be hydrolysed to release free fatty acids, or the oil hydrogenated or treated chemically or enzymatically as known in the art.

Rice oil after extraction from rice seed or bran typically comprises the group of lipids called γ-oryzanols. As used herein, “comprises γ-oryzanol” refers to the presence of at least 0.1% (w/w) γ-oryzanol compounds in the oil. The levels of γ-oryzanol in rice oil after extraction and before removal from the TAG is typically 1.5-3.5% (w/w). The compounds are typically a mixture of steryl and other triterpenyl esters of ferulic acid (4-hydroxy-3-methoxy cinnamic acid). Cycloartenyl ferulate, 24-methylene cycloartanyl ferulate and campesteryl ferulate are the predominant ferulates in oryzanol, with lower levels of β-sitosteryl ferulate and stigmasteryl ferulate. The presence of γ-oryzanols is thought to help protect consumers of rice oil against chronic diseases such as heart disease and cancer and therefore the presence of γ-oryzanol is advantageous.

As used herein, the “Rancimat” method is a well known test based on accelerated ageing. Air is conducted through the sample in the reaction vessel at a constantly increased temperature. The fatty acids are oxidized during this process. Volatile secondary reaction products are formed at the end of the test that are conducted by air flow into a measuring vessel, where they are absorbed by a measuring solution (distilled water). The continually recorded electrical conductivity increases as a result of the absorption of the ionic reaction products. The time up to which the secondary reaction products arise is called the induction time. It characterizes the oxidation stability of oils and fats.

As used herein, the term “rice bran” refers to the layer (aleurone layer) between the inner white rice grain and the outer hull of a rice seed/grain as well as the embryo/scutellum of the grain. The rice bran is the primary by product of the polishing of brown rice to produce white rice.

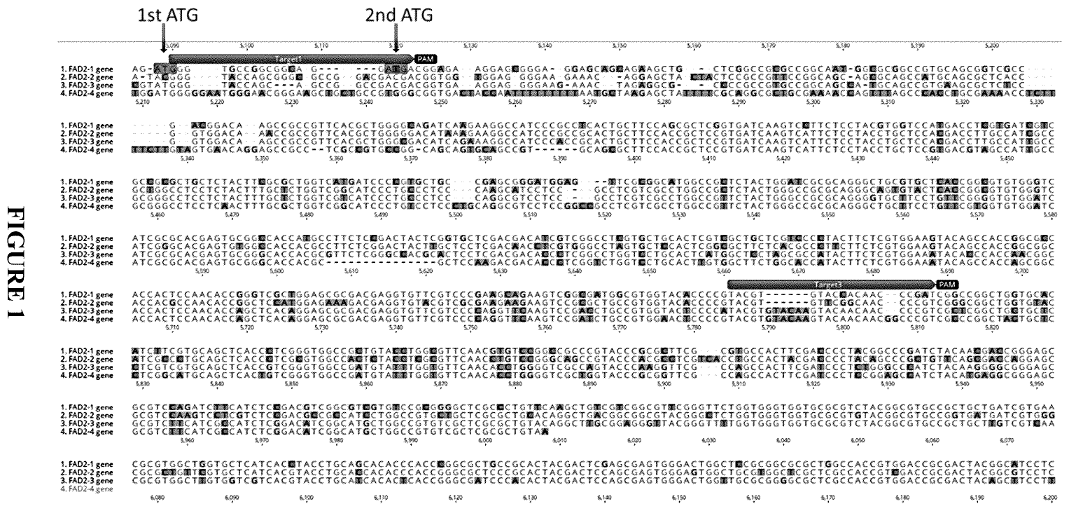

As used herein, the term “Fad2 protein” refers to a protein which performs a desaturase reaction converting oleic acid to linoleic acid. Thus, the term “Fad2 protein activity” refers to the conversion of oleic acid to linoleic acid. As used herein, the term “Fad2-1 protein” refers to an evolutionary conserved subclass of FAD2-1 proteins which are typically expressed in seeds. Examples of FAD 2-1 proteins have an amino acid sequence as set forth in any one of SEQ ID NO's 1 to 9. There are four Fad2 polypeptides in rice (Zaplin et al., 2013; WO 2008/006171), designated OsFAD2-1 (LOC_Os02g48560) (SEQ ID NO:1), OsFAD2-2 (LOC_Os07g23430) (SEQ ID NO:12), OsFAD2-3 (LOC_Os07g23410) (SEQ ID NO1:3) and OsFAD2-4 (LOC_Os07g23390) (SEQ ID NO:14) (FIG. 1). Naturally occurring FAD2 enzymes typically comprise three histidine-rich motifs which have been implicated in the formation of the diiron-oxygen complex used in biochemical catalysis (Shanklin et al., 1998). In an embodiment, a rice FAD2-1 protein has an amino acid sequence which is at least 95%, at least 97%, at least 99%, or at least 99.5% identical when compared to the sequence of amino acids set forth as SEQ ID NO:1, or is identical thereto.

As used herein, the term “LOX” or variations thereof refers to lipoxygenases (LOXs; EC 1.13.11.12) which catalyze peroxidation of lipids. Lipoxygenases have an amino terminal β-barrel, now known as a PLAT (Polycystin-1, Lipoxygenase, Alpha-Toxin) domain and a much larger α-helical domain that houses the catalytic iron. Lipoxygenases have an amino terminal β-barrel, now known as a PLAT (Polycystin-1, Lipoxygenase, Alpha-Toxin) domain and a much larger α-helical domain that houses the catalytic iron (Newcomer and Brash, 2015). LOXs are classified into three types (Mizuno et al., 2003). Type I lipoxygenase is localized in chloroplast and stress inducible; Type II lipoxygenase is localized in cytoplasm, derived from dicots, and is not stress inducible; Type III lipoxygenase is localized in the cytoplasm, derived from monocots and related to seed germination. Type I LOXs have a transit peptide, this is absent in Type II and Type III LOXs. LOXs are also classified as either 9-LOXs or 13-LOXs according to the enzymes preference for carbon 9 or carbon 13 in the substrate hydrocarbon backbone, generating 9(S)-hydroperoxy- and 9(S)-hydroperoxy-derivatives (Feussner and Wastemack, 2002). Based on bioinformatic analysis, it is purported that the rice genome (rice.plantbiology.msu.edu) has 14 LOX protein genes. Three isozymes of Type III LOXs (LOX1, LOX2, and LOX3) have been identified in developing rice seeds (Ohta et al., 1986). Among them, LOX3 is the most abundant enzyme. Examples of LOX3 proteins have an amino acid sequence as set forth in any one of SEQ ID NO's 23 to 29. In an embodiment, a rice LOX3 protein has an amino acid sequence which is at least 95%, at least 97%, at least 99%, or at least 99.5% identical when compared to the sequence of amino acids set forth as SEQ ID NO:23, or is identical thereof. As used herein, the term “LOX3 protein activity” refers to the peroxidation of fatty acids in cereal grain such as rice grain.

As used herein, the term “FatB polypeptide” refers to a protein which hydrolyses palmitoyl-ACP to produce free palmitic acid. Thus, the term “FatB activity” refers to the hydrolysis of palmitoyl-ACP to produce free palmitic acid. As used herein, the term “FatB-1 protein” refers to an evolutionary conserved subclass of FATB proteins which are typically expressed in seeds. There are four rice OsFATB genes were named FATB1 (LOC_Os06g05130) (SEQ ID NO:19), FATB2 (LOC_Os11g43820) (SEQ ID NO:20), FATB3 (LOC_Os02g43090) (SEQ ID NO:21) and FATB4 (LOC_Os06g39520) (SEQ ID NO:22) (WO 2008/006171). In an embodiment, a rice FATB-1 protein has an amino acid sequence which is at least 95%, at least 97%, at least 99%, or at least 99.5% identical when compared to the sequence of amino acids set forth as SEQ ID NO:21, or is identical thereto.

As used herein, the phrase “more stable” is a relative term. Stability refers the oxidative stability of the oil. In particular, a “more stable” oil (such as rice oil of the invention) is oxidised to a lesser extent than oil from a wild type plant (lacking the genetic modifications of the invention) when stored under the same conditions for the same length of time. As described herein, one measure for improved stability is hexanal production (see Example 9).

The terms “seed” and “grain” are used interchangeably herein. “Grain” generally refers to mature, harvested grain but can also refer to grain after imbibition or germination, according to the context. Mature grain commonly has a moisture content of less than about 18-20%.

As used herein, “fertile” grain is able to germinate to produce a fertile plant, whereas a fertile plant is able to produce fertile grain. In an embodiment, a plant of the invention is at least able to produce 50% or more, or 75% or more, of the amount of fertile grain when compared to a corresponding wild type plant lacking the genetic modifications.

“Wild type”, as used herein, refers to a cell, tissue or plant that has not been modified according to the invention. Wild-type cells, tissue or plants may be used as controls to compare levels of expression of an exogenous nucleic acid or the extent and nature of trait modification with cells, tissue or plants modified as described herein. Wild-type rice varieties that are suitable as a reference standard include Nipponbare.

### Polypeptides

The terms “polypeptide” and “protein” are generally used interchangeably.

By “substantially purified polypeptide” or “purified polypeptide” we mean a polypeptide that has generally been separated from the lipids, nucleic acids, other peptides, and other contaminating molecules with which it is associated in its native state. Preferably, the substantially purified polypeptide is at least 90% free from other components with which it is naturally associated. In an embodiment, the polypeptide of the invention has an amino acid sequence which is different to a naturally occurring FAD2-1 and/or LOX3 polypeptide i.e. is an amino acid sequence variant.

Genetically modified organisms, such as plants, and host cells of the invention may comprise an exogenous polynucleotide encoding a polypeptide of the invention. In these instances, the plants and cells produce a recombinant polypeptide. The term “recombinant” in the context of a polypeptide refers to the polypeptide encoded by an exogenous polynucleotide when produced by a cell, which polynucleotide has been introduced into the cell or a progenitor cell by recombinant DNA or RNA techniques such as, for example, transformation. Typically, the cell comprises a non-endogenous gene that causes an altered amount of the polypeptide to be produced. In an embodiment, a “recombinant polypeptide” is a polypeptide made by the expression of an exogenous (recombinant) polynucleotide in a plant cell.

The % identity of a polypeptide is determined by GAP (Needleman and Wunsch, 1970) analysis (GCG program) with a gap creation penalty=5, and a gap extension penalty=0.3. The query sequence is at least 300 amino acids in length, and the GAP analysis aligns the two sequences over a region of at least 300 amino acids. More preferably, the query sequence is at least 325 amino acids in length and the GAP analysis aligns the two sequences over a region of at least 335 amino acids. Even more preferably, the query sequence is at least 350 amino acids in length and the GAP analysis aligns the two sequences over a region of at least 350 amino acids. Even more preferably, the GAP analysis aligns two sequences over their entire length.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is preferably at least 96%, more preferably at least 97%, more preferably at least 98%, more preferably at least 99%, more preferably at least 99.10%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence mutants/variants of the polypeptides defined herein, particularly FAD2-1 and/or LOX3 mutants/variants, can be prepared by introducing appropriate nucleotide changes into a nucleic acid, or by in vitro synthesis of the desired polypeptide. Such mutants include, for example, deletions, insertions or substitutions of residues within the amino acid sequence. A combination of deletion, insertion and substitution can be made to arrive at the final construct, provided that the final peptide product possesses the desired characteristics. Preferred amino acid sequence mutants have one, two, three, four or less than 10 amino acid changes relative to the reference polypeptide. The mutant/variant may be N-terminally and/or C-terminally truncated.

In an embodiment, the FAD2-1 protein with reduced activity has an N-terminal truncation compared to the wild type sequence such as lacking the first three, four, five, or six N-terminal amino acids. In an embodiment, the mutant lacks the first six amino acids of the wild type FAD 2-1 protein. In an embodiment, the LOX3 protein with reduced, preferably no, activity has an C-terminal truncation compared to the wild type sequence such as lacking at least last 100, 200, 300, 400, 500, 600 or so C-terminal amino acids. In an embodiment, the mutant lacks the last about 500 C-terminal amino acids of the wild type LOX3 protein. In an embodiment, the genetically modified LOX3 gene only encodes the first about 91 amino acids of a wild type LOX3 protein.

Mutant (altered) polypeptides can be prepared using any technique known in the art, for example, using directed evolution, rational design strategies or mutagenesis (see below). Products derived from mutated/altered DNA can readily be screened using techniques described herein to determine if, when expressed in a plant, such as rice, confer reduced FAD 2-1 protein activity or LOX3 protein activity. For instance, the method may comprise producing a plant with a genetic modification expressing the mutated/altered DNA and determining the fertility and fatty acid profile of grain of the plant.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues, but may be even larger in the case of knockout mutants such as for LOX3.

Substitution mutants have at least one amino acid residue in the polypeptide molecule removed and a different residue inserted in its place. Where it is desirable to maintain a certain activity it is preferable to make more conservative substitutions at amino acid positions which are highly conserved in the relevant protein family. Examples of conservative substitutions are shown in Table 2 under the heading of “exemplary substitutions”.

TABLE 2
Exemplary substitutions.

| Original Residue | Exemplary Substitutions |
|---|---|
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

In an embodiment, a mutant/variant polypeptide has one or two or three or four conservative amino acid changes when compared to a naturally occurring polypeptide. Details of conservative amino acid changes are provided in Table 2.

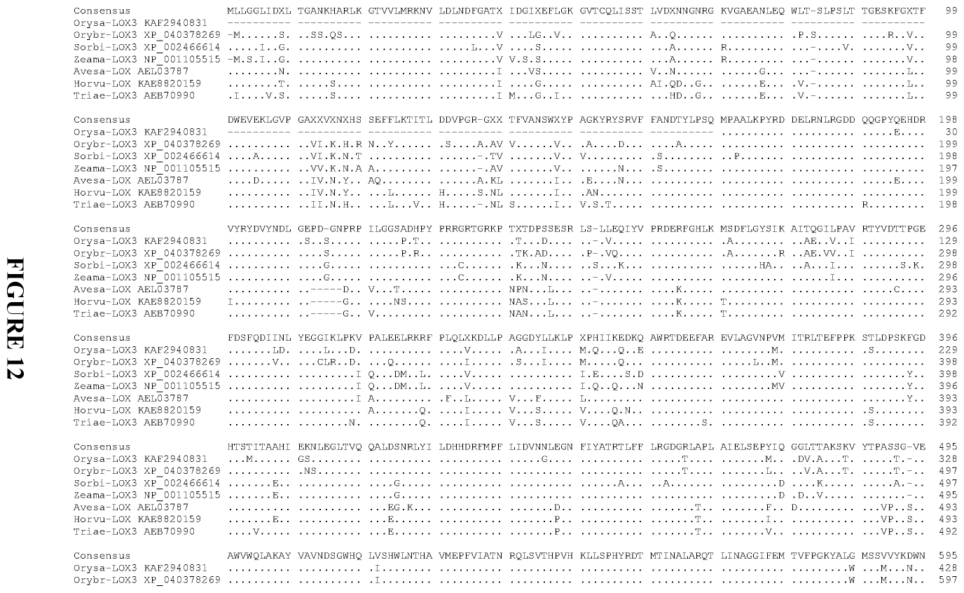

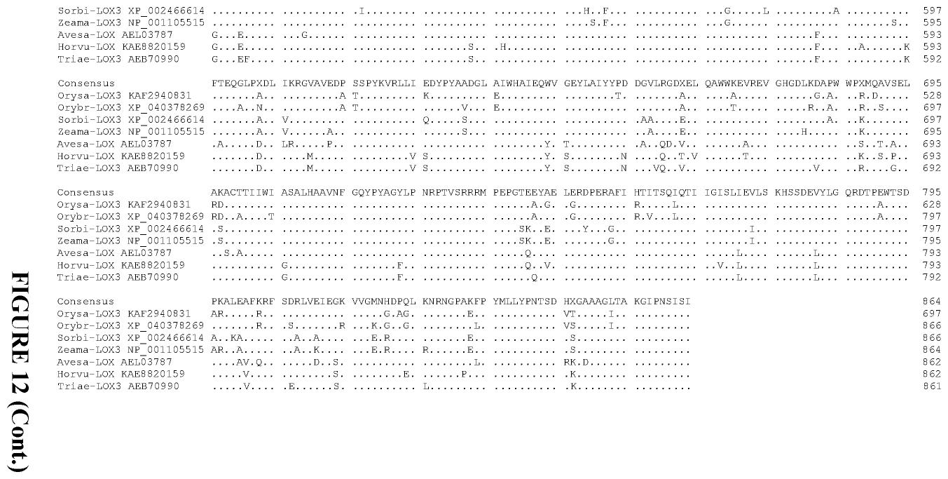

The primary amino acid sequence of wild-type polypeptides can be used to design variants/mutants thereof based on comparisons with closely related polypeptides (for example, as shown in FIGS. 11 and 12). As the skilled addressee will appreciate, residues highly conserved amongst closely related proteins are more likely to be able to be altered, especially with non-conservative substitutions, to reduce activity.

In an embodiment, the FATB1 with reduced activity has the amino acid sequence LNHVKTAG (SEQ ID NO:41) replaced with LNHVKTCW (SEQ ID NO:42). In an embodiment, the FATB1 with reduced activity has the amino acid sequence FLAAEKOW (SEQ ID NO:43) replaced with FLAAENSG (SEQ ID NO:44) or FLAAEKTV (SEQ ID NO:45). In an embodiment, the FATB1 with reduced activity has the amino acid sequence FLAAEKOW replaced with FLAAENSG.

In an embodiment, the FATB2 with reduced activity has the amino acid sequence MIRSYEIGAD (SEQ ID NO:46) replaced with MIRSYEDWC* (SEQ ID NO:47).

In an embodiment, the FATB3 with reduced activity has the amino acid sequence MIRSYEIGAD (SEQ ID NO:46) replaced with MIRSYEDWC* (SEQ ID NO:47) or MIRSYDWR* (SEQ ID NO:48). In an embodiment, the FATB3 with reduced activity has the amino acid sequence MIRSYEIGAD replaced with MIRSYEDWC*.

In an embodiment, the FATB4 with reduced activity has the amino acid sequence GLLGDGFG (SEQ ID NO:49) replaced with GLLGDFWL (SEQ ID NO:50), GLLGDGFW (SEQ ID NO:51), GLLGDFG (SEQ ID NO:52) or GLLFWLNA (SEQ ID NO:53). In an embodiment, the FATB4 with reduced activity has the amino acid sequence GLLGDGFG (SEQ ID NO:49) replaced with GLLGDFWL (SEQ ID NO:50).

In an embodiment, the FAD2-1 knockdown has the amino acid sequence MGAGGR (SEQ ID NO:54) deleted from the N-terminus. In an embodiment, the FAD2-1 knockdown has the amino acid sequence Aa177‘PYVYHNPIG’aa185 (SEQ ID NO:55) replaced with Aa177‘PYVYHTIG’aa184 (SEQ ID NO:56). In an embodiment, the FAD2-1 knockdown has the amino acid sequence MGAGGR deleted from the N-terminus and the amino acid sequence Aa177‘PYVYHNPIG’aa185 replaced with Aa177‘PYVYHTIG’aa184.

In an embodiment, the grain, bran and/or plant of the invention is homozygous for
i) LOX3 gene which only encodes the first about 91 amino acids of a wild type LOX3 protein,
ii) a FAD2-1 gene encoding a FAD2-1 polypeptide lacking MGAGGR at the N-terminus and comprising Aa177‘PYVYHTIG’aa184,
iii) a FATB2 gene encoding a polypeptide ending in MIRSYEDWC* (SEQ ID NO:47),
iv) a FATB3 gene encoding a polypeptide ending in MIRSYEDWC* (SEQ ID NO:47), and
v) a FATB4 gene encoding a polypeptide comprising GLLGDFWL.

In an embodiment, the grain, bran and/or plant of the invention is homozygous for
i) LOX3 gene which only encodes the first about 91 amino acids of a wild type LOX3 protein,
ii) a FAD2-1 gene encoding a FAD2-1 polypeptide lacking MGAGGR at the N-terminus and comprising Aa177‘PYVYHTIG’aa184,
iii) a FATB1 gene encoding a polypeptide comprising FLAAENSG, and
iv) a FATB4 gene encoding a polypeptide comprising GLLGDFWL.

In an embodiment, the grain, bran or plant has wild type phospholipase D (PLD) activity.

### Directed Evolution

In directed evolution, random mutagenesis is applied to a protein, and a selection regime is used to pick out variants that have the desired qualities, for example, decreased activity. Further rounds of mutation and selection are then applied. A typical directed evolution strategy involves three steps:
1) Diversification: The gene encoding the protein of interest is mutated and/or recombined at random to create a large library of gene variants. Variant gene libraries can be constructed through error prone P(CR (see, for example, Leung, 1989: Cadwxell and Joyce, 1992), from pools of DNaseI digested fragments prepared from parental templates (Stemmer, 1994a, Stemmer, 1994b; Crameri et al., 1998: Coco et al., 2001) from degenerate oligonucleotides (Ness et al., 2002, Coco, 2002) or from mixtures of both, or even from undigested parental templates (Zhao et al., 1998; Eggert et al., 2005; Jézéquek et al., 2008) and are usually assembled through PCR. Libraries can also be made from parental sequences recombined in vivo or in vitro by either homologous or non-homologous recombination (Ostermeier et al., 1999; Volkov et al., 1999; Sieber et al., 2001). Variant gene libraries can also be constructed by sub-cloning a gene of interest into a suitable vector, transforming the vector into a “mutator” strain such as the E. coli XL-1 red (Stratagene) and propagating the transformed bacteria for a suitable number of generations. Variant gene libraries can also be constructed by subjecting the gene of interest to DNA shuffling (i.e., in vitro homologous recombination of pools of selected mutant genes by random fragmentation and reassembly) as broadly described by Harayama (1998).
2) Selection: The library is tested for the presence of mutants (variants) possessing the desired property using a screen or selection. Screens enable the identification and isolation of high-performing mutants by hand, while selections automatically eliminate all nonfunctional mutants. A screen may involve screening for the presence of known conserved amino acid motifs. Alternatively, or in addition, a screen may involve expressing the mutated polynucleotide in a host organism or part thereof and assaying the level of activity.
3) Amplification: The variants identified in the selection or screen are replicated many fold, enabling researchers to sequence their DNA in order to understand what mutations have occurred.

Together, these three steps are termed a “round” of directed evolution. Most experiments will entail more than one round. In these experiments, the “winners” of the previous round are diversified in the next round to create a new library. At the end of the experiment, all evolved protein or polynucleotide mutants are characterized using biochemical methods.

### Rational Design

A protein can be designed rationally, on the basis of known information about protein structure and folding. This can be accomplished by design from scratch (de novo design) or by redesign based on native scaffolds (see, for example, Hellinga, 1997; and Lu and Berry, Protein Structure Design and Engineering, Handbook of Proteins 2, 1153-1157 (2007)). Protein design typically involves identifying sequences that fold into a given or target structure and can be accomplished using computer models. Computational protein design algorithms search the sequence-conformation space for sequences that are low in energy when folded to the target structure. Computational protein design algorithms use models of protein energetics to evaluate how mutations would affect a protein's structure and function. These energy functions typically include a combination of molecular mechanics, statistical (i.e. knowledge-based), and other empirical terms. Suitable available software includes IPRO (Interative Protein Redesign and Optimization), EGAD (A Genetic Algorithm for Protein Design), Rosetta Design, Sharpen, and Abalone.

### Polynucleotides and Genes

The present invention refers to various polynucleotides. As used herein, a “polynucleotide” or “nucleic acid” or “nucleic acid molecule” means a polymer of nucleotides, which may be DNA or RNA or a combination thereof, and includes genomic DNA, mRNA, cRNA, and cDNA. Less preferred polynucleotides include tRNA, siRNA, shRNA and hpRNA. It may be DNA or RNA of cellular, genomic or synthetic origin, for example made on an automated synthesizer, and may be combined with carbohydrate, lipids, protein or other materials, labelled with fluorescent or other groups, or attached to a solid support to perform a particular activity defined herein, or comprise one or more modified nucleotides not found in nature, well known to those skilled in the art. The polymer may be single-stranded, essentially double-stranded or partly double-stranded. Basepairing as used herein refers to standard basepairing between nucleotides, including G:U basepairs. “Complementary” means two polynucleotides are capable of basepairing (hybridizing) along part of their lengths, or along the full length of one or both. The term “polynucleotide” is used interchangeably herein with the term “nucleic acid”. Preferred polynucleotides of the invention encode a polypeptide of the invention.

By “isolated polynucleotide” we mean a polynucleotide which has generally been separated from the polynucleotide sequences with which it is associated or linked in its native state, if the polynucleotide is found in nature. Preferably, the isolated polynucleotide is at least 90% free from other components with which it is naturally associated, if it is found in nature. Preferably the polynucleotide is not naturally occurring, for example by covalently joining two shorter polynucleotide sequences in a manner not found in nature (chimeric polynucleotide).

The present invention may involve the modification of gene activity and the construction and use of chimeric genes. As used herein, the term “gene” includes any deoxyribonucleotide sequence which includes a protein coding region or which is transcribed in a cell but not translated, as well as associated non-coding and regulatory regions. Such associated regions are typically located adjacent to the coding region or the transcribed region on both the 5′ and 3′ ends for a distance of about 2 kb on either side. In this regard, the gene may include control signals such as promoters, enhancers, termination and/or polyadenylation signals that are naturally associated with a given gene, or heterologous control signals in which case the gene is referred to as a “chimeric gene”. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene.

A genomic form or clone of a gene containing the transcribed region may be interrupted with non-coding sequences termed “introns” or “intervening regions” or “intervening sequences”, which may be either homologous or heterologous with respect to the “exons” of the gene. An “intron” as used herein is a segment of a gene which is transcribed as part of a primary RNA transcript but is not present in the mature mRNA molecule. Introns are removed or “spliced out” from the nuclear or primary transcript; introns therefore are absent in the messenger RNA (mRNA). Introns may contain regulatory elements such as enhancers. “Exons” as used herein refer to the DNA regions corresponding to the RNA sequences which are present in the mature mRNA or the mature RNA molecule in cases where the RNA molecule is not translated. An mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above. A gene may be introduced into an appropriate vector for extrachromosomal maintenance in a cell or, preferably, for integration into the host genome.

As used herein, a “chimeric gene” refers to any gene that comprises covalently joined sequences that are not found joined in nature. Typically, a chimeric gene comprises regulatory and transcribed or protein coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. In an embodiment, the protein coding region is operably linked to a promoter or polyadenylation/terminator region which is heterologous to the gene, thereby forming a chimeric gene. The term “endogenous” is used herein to refer to a substance that is normally present or produced in an unmodified plant at the same developmental stage as the plant under investigation. An “endogenous gene” refers to a native gene in its natural location in the genome of an organism. As used herein, “recombinant nucleic acid molecule”, “recombinant polynucleotide” or variations thereof refer to a nucleic acid molecule which has been constructed or modified by recombinant DNA/RNA technology. The terms “foreign polynucleotide” or “exogenous polynucleotide” or “heterologous polynucleotide” and the like refer to any nucleic acid which is introduced into the genome of a cell by experimental manipulations.

Foreign or exogenous genes may be genes that are inserted into a non-native organism or cell, native genes introduced into a new location within the native host, or chimeric genes. Alternatively, foreign or exogenous genes may be the result of editing the genome of the organism or cell, or progeny derived therefrom. A “transgene” is a gene that has been introduced into the genome by a transformation procedure.

Furthermore, the term “exogenous” in the context of a polynucleotide (nucleic acid) refers to the polynucleotide when present in a cell that does not naturally comprise the polynucleotide.

The % identity of a polynucleotide is determined by GAP (Needleman and Wunsch, 1970) analysis (GCG program) with a gap creation penalty=5, and a gap extension penalty=0.3. The query sequence is at least 900 nucleotides in length, and the GAP analysis aligns the two sequences over a region of at least 900 nucleotides. Preferably, the query sequence is at least 975 nucleotides in length, and the GAP analysis aligns the two sequences over a region of at least 975 nucleotides. Even more preferably, the query sequence is at least 1,050 nucleotides in length and the GAP analysis aligns the two sequences over a region of at least 1,050 nucleotides. Even more preferably, the GAP analysis aligns two sequences over their entire length.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 50%, at least 60%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.10%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

The present invention also relates to the use of oligonucleotides, for instance in methods of screening for a polynucleotide of, or encoding a polypeptide of, the invention. As used herein, “oligonucleotides” are polynucleotides up to 50 nucleotides in length. The minimum size of such oligonucleotides is the size required for the formation of a stable hybrid between an oligonucleotide and a complementary sequence on a nucleic acid molecule of the present invention. They can be RNA, DNA, or combinations or derivatives of either. Oligonucleotides are typically relatively short single stranded molecules of 10 to 30 nucleotides, commonly 15-25 nucleotides in length. When used as a guide for genome editing, probe or as a primer in an amplification reaction, the minimum size of such an oligonucleotide is the size required for the formation of a stable hybrid between the oligonucleotide and a complementary sequence on a target nucleic acid molecule. Preferably, the oligonucleotides are at least 15 nucleotides, more preferably at least 18 nucleotides, more preferably at least 19 nucleotides, more preferably at least 20 nucleotides, more preferably at least 22 nucleotides, even more preferably at least 25 nucleotides in length. Oligonucleotides of the present invention used as a probe are typically conjugated with a label such as a radioisotope, an enzyme, biotin, a fluorescent molecule or a chemiluminescent molecule.

As those skilled in the art would be aware, the sequence of the oligonucleotide primers described herein can be varied to some degree without effecting their usefulness for the methods of the invention. A “variant” of an oligonucleotide disclosed herein (also referred to herein as a “primer” or “probe” depending on its use) useful for the methods of the invention includes molecules of varying sizes of, and/or are capable of hybridising to the genome close to that of, the specific oligonucleotide molecules defined herein. For example, variants may comprise additional nucleotides (such as 1, 2, 3, 4, or more), or less nucleotides as long as they still hybridise to the target region. Furthermore, a few nucleotides may be substituted without influencing the ability of the oligonucleotide to hybridise the target region. In addition, variants may readily be designed which hybridise close (for example, but not limited to, within 50 nucleotides or within 100 nucleotides) to the region of the genome where the specific oligonucleotides defined herein hybridise.

The present invention includes oligonucleotides that can be used as, for example, guides for RNA-guided endonucleases (see, for examples SEQ ID NO's 30 to 37), probes to identify nucleic acid molecules, or primers to produce nucleic acid molecules. Probes and/or primers can be used to clone homologues of the polynucleotides of the invention from other species. Furthermore, hybridization techniques known in the art can also be used to screen genomic or cDNA libraries for such homologues.

Polynucleotides of the present invention possess, when compared to naturally occurring molecules, one or more genetic modifications which are deletions, insertions, or substitutions of nucleotide residues. A variant of a polynucleotide or an oligonucleotide of the invention includes molecules of varying sizes of, and/or are capable of hybridising to, the rice (for example) genome close to that of the reference polynucleotide or oligonucleotide molecules defined herein. For example, variants may comprise additional nucleotides (such as 1, 2, 3, 4, or more), or less nucleotides as long as they still hybridise to the target region. Furthermore, a few nucleotides may be substituted without influencing the ability of the oligonucleotide to hybridise to the target region. In addition, variants may readily be designed which hybridise close to, for example to within 50 nucleotides, the region of the plant genome where the specific oligonucleotides defined herein hybridise. In particular, this includes polynucleotides which encode the same polypeptide or amino acid sequence but which vary in nucleotide sequence by redundancy of the genetic code. The terms “polynucleotide variant” and “variant” also include naturally occurring allelic variants.

### Nucleic Acid Constructs

The present invention includes nucleic acid constructs comprising the polynucleotides of the invention, and vectors and host cells containing these, methods of their production and use, and uses thereof. The present invention refers to elements which are operably connected or linked. “Operably connected” or “operably linked” and the like refer to a linkage of polynucleotide elements in a functional relationship. Typically, operably connected nucleic acid sequences are contiguously linked and, where necessary to join two protein coding regions, contiguous and in reading frame. A coding sequence is “operably connected to” another coding sequence when RNA polymerase will transcribe the two coding sequences into a single RNA, which if translated is then translated into a single polypeptide having amino acids derived from both coding sequences. The coding sequences need not be contiguous to one another so long as the expressed sequences are ultimately processed to produce the desired protein.

As used herein, the term “cis-acting sequence”, “cis-acting element” or “cis-regulatory region” or “regulatory region” or similar term shall be taken to mean any sequence of nucleotides, which when positioned appropriately and connected relative to an expressible genetic sequence, is capable of regulating, at least in part, the expression of the genetic sequence. Those skilled in the art will be aware that a cis-regulatory region may be capable of activating, silencing, enhancing, repressing or otherwise altering the level of expression and/or cell-type-specificity and/or developmental specificity of a gene sequence at the transcriptional or post-transcriptional level. In preferred embodiments of the present invention, the cis-acting sequence is an activator sequence that enhances or stimulates the expression of an expressible genetic sequence.

“Operably connecting” a promoter or enhancer element to a transcribable polynucleotide means placing the transcribable polynucleotide (e.g., protein-encoding polynucleotide or other transcript) under the regulatory control of a promoter, which then controls the transcription of that polynucleotide. In the construction of heterologous promoter/structural gene combinations, it is generally preferred to position a promoter or variant thereof at a distance from the transcription start site of the transcribable polynucleotide which is approximately the same as the distance between that promoter and the protein coding region it controls in its natural setting; i.e., the gene from which the promoter is derived. As is known in the art, some variation in this distance can be accommodated without loss of function. Similarly, the preferred positioning of a regulatory sequence element (e.g., an operator, enhancer etc) with respect to a transcribable polynucleotide to be placed under its control is defined by the positioning of the element in its natural setting; i.e., the genes from which it is derived.

“Promoter” or “promoter sequence” as used herein refers to a region of a gene, generally upstream (5′) of the RNA encoding region, which controls the initiation and level of transcription in the cell of interest. A “promoter” includes the transcriptional regulatory sequences of a classical genomic gene, such as a TATA box and CCAAT box sequences, as well as additional regulatory elements (i.e., upstream activating sequences, enhancers and silencers) that alter gene expression in response to developmental and/or environmental stimuli, or in a tissue-specific or cell-type-specific manner. A promoter is usually, but not necessarily (for example, some PolIII promoters), positioned upstream of a structural gene, the expression of which it regulates. Furthermore, the regulatory elements comprising a promoter are usually positioned within 2 kb of the start site of transcription of the gene. Promoters may contain additional specific regulatory elements, located more distal to the start site to further enhance expression in a cell, and/or to alter the timing or inducibility of expression of a structural gene to which it is operably connected.

“Constitutive promoter” refers to a promoter that directs expression of an operably linked transcribed sequence in many or all tissues of an organism such as a plant. The term constitutive as used herein does not necessarily indicate that a gene is expressed at the same level in all cell types, but that the gene is expressed in a wide range of cell types, although some variation in level is often detectable. “Selective expression” as used herein refers to expression almost exclusively in specific organs of, for example, the plant, such as, for example, endosperm, embryo, leaves, fruit, tubers or root. In a preferred embodiment, a promoter is expressed selectively or preferentially in leaves and/or stems of a plant, preferably a cereal plant. Selective expression may therefore be contrasted with constitutive expression, which refers to expression in many or all tissues of a plant under most or all of the conditions experienced by the plant.

Selective expression may also result in compartmentation of the products of gene expression in specific plant tissues, organs or developmental stages such as adults or seedlings. Compartmentation in specific subcellular locations such as the plastid, cytosol, vacuole, or apoplastic space may be achieved by the inclusion in the structure of the gene product of appropriate signals, eg. a signal peptide, for transport to the required cellular compartment, or in the case of the semi-autonomous organelles (plastids and mitochondria) by integration of a transgene with appropriate regulatory sequences directly into the organelle genome.

A “tissue-specific promoter” or “organ-specific promoter” is a promoter that is preferentially expressed in one tissue or organ relative to many other tissues or organs, preferably most if not all other tissues or organs in, for example, a plant. Typically, the promoter is expressed at a level 10-fold higher in the specific tissue or organ than in other tissues or organs.

The promoters contemplated by the present invention may be native to the host plant to be transformed or may be derived from an alternative source, where the region is functional in the host plant. Other sources include the Agrobacterium T-DNA genes, such as the promoters of genes for the biosynthesis of nopaline, octapine, mannopine, or other opine promoters, tissue specific promoters (see, e.g., U.S. Pat. No. 5,459,252 and WO 91/13992); promoters from viruses (including host specific viruses), or partially or wholly synthetic promoters. Numerous promoters that are functional in mono- and dicotyledonous plants are well known in the art (see, for example, Greve, 1983; Salomon et al., 1984; Garfinkel et al., 1983; Barker et al., 1983); including various promoters isolated from plants and viruses such as the cauliflower mosaic virus promoter (CaMV 355, 19S). Non-limiting methods for assessing promoter activity are disclosed by Medberry et al. (1992, 1993), Sambrook et al. (1989, supra) and U.S. Pat. No. 5,164,316.

Alternatively, or additionally, the promoter may be an inducible promoter or a developmentally regulated promoter which is capable of driving expression of the introduced polynucleotide at an appropriate developmental stage of the, for example, plant. Other cis-acting sequences which may be employed include transcriptional and/or translational enhancers. Enhancer regions are well known to persons skilled in the art, and can include an ATG translational initiation codon and adjacent sequences. When included, the initiation codon should be in phase with the reading frame of the coding sequence relating to the foreign or exogenous polynucleotide to ensure translation of the entire sequence if it is to be translated. Translational initiation regions may be provided from the source of the transcriptional initiation region, or from a foreign or exogenous polynucleotide. The sequence can also be derived from the source of the promoter selected to drive transcription, and can be specifically modified so as to increase translation of the mRNA.

The nucleic acid construct of the present invention may comprise a 3′ non-translated sequence from about 50 to 1,000 nucleotide base pairs which may include a transcription termination sequence. A 3′ non-translated sequence may contain a transcription termination signal which may or may not include a polyadenylation signal and any other regulatory signals capable of effecting mRNA processing. A polyadenylation signal functions for addition of polyadenylic acid tracts to the 3′ end of a mRNA precursor. Polyadenylation signals are commonly recognized by the presence of homology to the canonical form 5′ AATAAA-3′ although variations are not uncommon. Transcription termination sequences which do not include a polyadenylation signal include terminators for Poll or PolIII RNA polymerase which comprise a run of four or more thymidines. Examples of suitable 3′ non-translated sequences are the 3′ transcribed non-translated regions containing a polyadenylation signal from an octopine synthase (ocs) gene or nopaline synthase (nos) gene of Agrobacterium tumefaciens (Bevan et al., 1983). Suitable 3′ non-translated sequences may also be derived from plant genes such as the ribulose-1,5-bisphosphate carboxylase (ssRUBISCO) gene, although other 3′ elements known to those of skill in the art can also be employed.

As the DNA sequence inserted between the transcription initiation site and the start of the coding sequence, i.e., the untranslated 5′ leader sequence (5′UTR), can influence gene expression if it is translated as well as transcribed, one can also employ a particular leader sequence. Suitable leader sequences include those that comprise sequences selected to direct optimum expression of the foreign or endogenous DNA sequence. For example, such leader sequences include a preferred consensus sequence which can increase or maintain mRNA stability and prevent inappropriate initiation of translation as for example described by Joshi (1987).

### Vectors

The present invention includes use of vectors for manipulation or transfer of genetic constructs. By “vector” or “chimeric vector” is meant a nucleic acid molecule, preferably a DNA molecule derived, for example, from a plasmid, bacteriophage, or plant virus, into which a nucleic acid sequence may be inserted or cloned. A vector preferably is double-stranded DNA and contains one or more unique restriction sites and may be capable of autonomous replication in a defined host cell including a target cell or tissue or a progenitor cell or tissue thereof, or capable of integration into the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into a cell, is integrated into the genome of the recipient cell and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the cell into which the vector is to be introduced. The vector may also include a selection marker such as an antibiotic resistance gene, a herbicide resistance gene or other gene that can be used for selection of suitable transformants. Examples of such genes are well known to those of skill in the art.

The nucleic acid construct of the invention can be introduced into a vector, such as a plasmid. Plasmid vectors typically include additional nucleic acid sequences that provide for easy selection, amplification, and transformation of the expression cassette in prokaryotic and eukaryotic cells, e.g., pUC-derived vectors, pSK-derived vectors, pGEM-derived vectors, pSP-derived vectors, pBS-derived vectors, or binary vectors containing one or more T-DNA regions. Additional nucleic acid sequences include origins of replication to provide for autonomous replication of the vector, selectable marker genes, preferably encoding antibiotic or herbicide resistance, unique multiple cloning sites providing for multiple sites to insert nucleic acid sequences or genes encoded in the nucleic acid construct, and sequences that enhance transformation of prokaryotic and eukaryotic (especially plant) cells.

By “marker gene” is meant a gene that imparts a distinct phenotype to cells expressing the marker gene and thus allows such transformed cells to be distinguished from cells that do not have the marker. A selectable marker gene confers a trait for which one can “select” based on resistance to a selective agent (e.g., a herbicide, antibiotic, radiation, heat, or other treatment damaging to untransformed cells). A screenable marker gene (or reporter gene) confers a trait that one can identify through observation or testing, i.e., by “screening” (e.g., β-glucuronidase, luciferase, GFP or other enzyme activity not present in untransformed cells). The marker gene and the nucleotide sequence of interest do not have to be linked.

To facilitate identification of transformants, the nucleic acid construct desirably comprises a selectable or screenable marker gene as, or in addition to, the foreign or exogenous polynucleotide. The actual choice of a marker is not crucial as long as it is functional (i.e., selective) in combination with the plant cells of choice. The marker gene and the foreign or exogenous polynucleotide of interest do not have to be linked, since co-transformation of unlinked genes as, for example, described in U.S. Pat. No. 4,399,216 is also an efficient process in plant transformation.

Examples of bacterial selectable markers are markers that confer antibiotic resistance such as ampicillin, erythromycin, chloramphenicol or tetracycline resistance, preferably kanamycin resistance. Exemplary selectable markers for selection of plant transformants include, but are not limited to, a hyg gene which encodes hygromycin B resistance; a neomycin phosphotransferase (nptII) gene conferring resistance to kanamycin, paromomycin, G418; a glutathione-S-transferase gene from rat liver conferring resistance to glutathione derived herbicides as, for example, described in EP 256223; a glutamine synthetase gene conferring, upon overexpression, resistance to glutamine synthetase inhibitors such as phosphinothricin as, for example, described in WO 87/05327, an acetyltransferase gene from Streptomyces viridochromogenes conferring resistance to the selective agent phosphinothricin as, for example, described in EP 275957, a gene encoding a 5-enolshikimate-3-phosphate synthase (EPSPS) conferring tolerance to N-phosphonomethylglycine as, for example, described by Hinchee et al. (1988), a bar gene conferring resistance against bialaphos as, for example, described in WO91/02071; a nitrilase gene such as bxn from Klebsiella ozaenae which confers resistance to bromoxynil (Stalker et al., 1988); a dihydrofolate reductase (DHFR) gene conferring resistance to methotrexate (Thillet et al., 1988); a mutant acetolactate synthase gene (ALS), which confers resistance to imidazolinone, sulfonylurea or other ALS-inhibiting chemicals (EP 154,204); a mutated anthranilate synthase gene that confers resistance to 5-methyl tryptophan; or a dalapon dehalogenase gene that confers resistance to the herbicide.

Preferred screenable markers include, but are not limited to, a uidA gene encoding a β-glucuronidase (GUS) enzyme for which various chromogenic substrates are known, a β-galactosidase gene encoding an enzyme for which chromogenic substrates are known, an aequorin gene (Prasher et al., 1985), which may be employed in calcium-sensitive bioluminescence detection; a green fluorescent protein gene or derivatives thereof; a luciferase (luc) gene (Ow et al., 1986), which allows for bioluminescence detection, and others known in the art. By “reporter molecule” as used in the present specification is meant a molecule that, by its chemical nature, provides an analytically identifiable signal that facilitates determination of promoter activity by reference to protein product.

Preferably, the nucleic acid construct is stably incorporated into the genome of, for example, the plant. Accordingly, the nucleic acid comprises appropriate elements which allow the molecule to be incorporated into the genome, or the construct is placed in an appropriate vector which can be incorporated into a chromosome of a plant cell.

One embodiment of the present invention includes a recombinant vector, which includes at least one polynucleotide molecule of the present invention, inserted into any vector capable of delivering the nucleic acid molecule into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid.

A number of vectors suitable for stable transfection of plant cells or for the establishment of genetically modified plants have been described in, e.g., Pouwels et al., Cloning Vectors: A Laboratory Manual, 1985, supp. 1987; Weissbach and Weissbach, Methods for Plant Molecular Biology, Academic Press, 1989; and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

### Recombinant Cells

Another embodiment of the present invention includes a recombinant cell comprising a host cell transformed with one or more recombinant molecules of the present invention, or progeny cells thereof. Transformation of a nucleic acid molecule into a cell can be accomplished by any method by which a nucleic acid molecule can be inserted into the cell. Transformation techniques include, but are not limited to, transfection, particle bombardment/biolistics, electroporation, microinjection, lipofection, adsorption, and protoplast fusion. In an embodiment, gene editing is used to transform the target cell using, for example, targeting nucleases such as TALEN, Cpf1, MAD7 and Cas9-CRISPR or engineered nucleases derived therefrom.

A recombinant cell may remain unicellular or may grow into a tissue, organ or a multicellular organism. Transformed nucleic acid molecules of the present invention can remain extrachromosomal or can integrate into one or more sites within a chromosome of the transformed (i.e., recombinant) cell in such a manner that their ability to be expressed is retained. Preferred host cells are plant cells, more preferably cells of a cereal plant, more preferably rice or sorghum cells, and even more preferably a rice cell.

### Genome Editing

Endonucleases can be used to generate single strand or double strand breaks in genomic DNA. The genomic DNA breaks in eukaryotic cells are repaired using non-homologous end joining (NHEJ) or homology directed repair (HDR) pathways. NHEJ may result in imperfect repair resulting in unwanted mutations and HDR can enable precise gene insertion by using an exogenous supplied repair DNA template. CRISPR-associated (Cas) proteins have received significant interest although transcription activator-like effector nucleases (TALENs) and zinc-finger nucleases are still useful, the CRISPR-Cas system offers a simpler, versatile and cheaper tool for genome modification (Doudna and Charpentier, 2014).

The CRISPR-Cas systems are classed into three major groups using various nucleases or combinations on nuclease. In class 1 CRISPR-Cas systems (types I, III and IV), the effector module consists of a multi-protein complex whereas class 2 systems (types II, V and VI) use only one effector protein (Makarova et al., 2015). Cas includes a gene that is coupled or close to or localised near the flanking CRISPR loci. Haft et al. (2005) provides a review of the Cas protein family.

The nuclease is guided by the synthetic small guide RNA (sgRNAs or gRNAs) that may or may not include the tracRNA resulting in a simplification of the CRISPR-Cas system to two genes; the endonuclease and the sgRNA (Jinek et al. 2012). The sgRNA is typically under the regulatory control of a U3 or U6 small nuclear RNA promoter. The sgRNA recognises the specific gene and part of the gene for targeting. The protospacer adjacent motif (PAM) is adjacent to the target site constraining the number of potential CRISPR-Cas targets in a genome although the expansion of nucleases also increases the number of PAM's available. There are numerous web tools available for designing gRNAs including CHOPCHOP (http://chopchop.cbu.uib.no), CRISPR design https://omictools.com/crispr-design-tool, E-CRISP http://www.e-crisp.org/E-CRISP/, Geneious or Benchling https://benchling.com/crispr.

CRISPR-Cas systems are the most frequently adopted in eukaryotic work to date using a Cas9 effector protein typically using the RNA-guided Streptococcus pyogenes Cas9 or an optimised sequence variant in multiple plant species (Luo et al., 2016). Luo et al. (2016) summarises numerous studies where genes have been successfully targeted in various plant species to give rise to indels and loss of function mutant phenotypes in the endogenous gene open reading frame and/or promoter. Due to the cell wall on plant cells the delivery of the CRISPR-Cas machinery into the cell and successful transgenic regenerations have used Agrobacterium tumefaciens infection (Luo et al., 2016) or plasmid DNA particle bombardment or biolistic delivery. Vectors suitable for cereal transformation include pCXUNcas9 (Sun et al, 2016) or pYLCRISPR/Cas9Pubi-H available from Addgene (Ma et al., 2015, accession number KR029109.1).

Alternative CRISPR-Cas systems refer to effector enzymes that contain the nuclease RuvC domain but do not contain the HNH domain including Cas12 enzymes including Cas12a, Cas12b, Cas12f, Cpf1, C2c1, C2c3, and engineered derivatives. Cpf1 creates double-stranded breaks in a staggered manner at the PAM-distal position and being a smaller endonuclease may provide advantages for certain species (Begemann et al., 2017). Other CRISPR-Cas systems include RNA-guided RNAses including Cas13, Cas13a (C2c2), Cas13b, Cas13c.

### Sequence Insertion or Integration

The CRISPR-Cas system can be combined with the provision of a nucleic acid sequence to direct homologous repair for the insertion of a sequence into a genome. Targeted genome integration of plant transgenes enables the sequential addition of transgenes at the same locus. This “cis gene stacking” would greatly simplify subsequent breeding efforts with all transgenes inherited as a single locus. When coupled with CRISPR/Cas9 cleavage of the target site the transgene can be incorporated into this locus by homology-directed repair that is facilitated by flanking sequence homology. This approach can be used to rapidly introduce new alleles without linkage drag or to introduce allelic variants that do not exist naturally.

### Nickases

The CRISPR-Cas II systems use a Cas9 nuclease with two enzymatic cleavage domains a RuvC and HNH domain. Mutations have been shown to alter the double strand cutting to single strand cutting and resulting in a technology variant referred to as a nickase or a nuclease-inactivated Cas9. The RuvC subdomain cleaves the non-complementary DNA strand and the HNH subdomain cleaves that DNA strand complementary to the gRNA. The nickase or nuclease-inactivated Cas9 retains DNA binding ability directed by the gRNA. Mutations in the subdomains are known in the art for example S. pyogenes Cas9 nuclease with a D10A mutation or H840A mutation.

### Genome Base Editing or Modification

Base editors have been created by fusing a deaminase with a Cas9 domain (WO 2018/086623). By fusing the deaminase can take advantage of the sequence targeting directed by the gRNA to make targeted cytidine (C) to uracil (U) conversion by deamination of the cytidine in the DNA. The mismatch repair mechanisms of the cell then replace the U with a T. Suitable cytidine deaminases may include APOBEC1 deaminase, activation-induced cytidine deaminase (AID), APOBEC3G and CDA1. Further, the Cas9-deaminase fusion may be a mutated Cas9 with nickase activity to generate a single strand break. It has been suggested that the nickase protein was potentially more efficient in promoting homology-directed repair (Luo et al., 2016).

### Vector Free Genome Editing or Genome Modification

More recently methods to use vector free approaches using Cas9/sgRNA ribonucleoproteins have been described with successful reduction of off-target events. The method requires in vitro expression of Cas9 ribonucleoproteins (RNPs) which are transformed into the cell or protoplast and does not rely on the Cas9 being integrated into the host genome, thereby reducing the undesirable side cuts that has been linked with the random integration of the Cas9 gene. Only short flanking sequences are required to form a stable Cas9 and sgRNA stable ribonucleoprotein in vitro. Woo et al. (2015) produced pre-assembled Cas9/sgRNA protein/RNA complexes and introduced them into protoplasts of Arabidopsis, rice, lettuce and tobacco and targeted mutagenesis frequencies of up to 45% observed in regenerated plants. RNP and in vitro demonstrated in several species including dicot plants (Woo et al., 2015), and monocots maize (Svitashev et al., 2016) and wheat (Liang et al., 2017). Genome editing of plants using CRISPR-Cas 9 in vitro transcripts or ribonucleoproteins are fully described in Liang et al. (2018) and Liang et al. (2019).

### Method for Gene Insertion

Plant embryos may be bombarded with a Cas9 gene and sgRNA gene targeting the site of integration along with the DNA repair template. DNA repair templates are may be synthesised DNA fragment or a 127-mer oligonucleotide, with each encoding the cDNA or the gene of interest. Bombarded cells are grown on tissue culture medium. DNA extracted from callus or TO plants leaf tissue using CTAB DNA extraction method can be analysed by PCR to confirm gene integration. T1 plants selected if pcr confirms presence of the gene of interest.

The method comprises introducing into a plant cell the DNA sequence of interest referred to as the donor DNA and the endonuclease. The endonuclease generates a break in the target site allowing the first and second regions of homology of the donor DNA to undergo homologous recombination with their corresponding genomic regions of homology. The cut genomic DNA acts as an acceptor of the DNA sequence. The resulting exchange of DNA between the donor and the genome results in the integration of the polynucleotide of interest of the donor DNA into the strand break in the target site in the plant genome, thereby altering the original target site and producing an altered genomic sequence.

The donor DNA may be introduced by any means known in the art. For example, a plant having a target site is provided. The donor DNA may be provided to the plant by known transformation methods including, Agrobacterium-mediated transformation or biolistic particle bombardment. The RNA guided Cas or Cpf1 endonuclease cleaves at the target site, the donor DNA is inserted into the transformed plant's genome.

Although homologous recombination occurs at low frequency in plant somatic cells the process appears to be increased/stimulated by the introduction of doublestrand breaks (DSBs) at selected endonuclease target sites. Ongoing efforts to generate Cas, in particular Cas9, and Cas12a/Cpf1, variants or alternatives such as MAD7 or Cms1 may improve the efficiency.

### Genetically Modified Plants

The term “plant” as used herein as a noun refers to whole plants and refers to any member of the Kingdom Plantae, but as used as an adjective refers to any substance which is present in, obtained from, derived from, or related to a plant, such as for example, plant organs (e.g. leaves, stems, roots, flowers), single cells (e.g. pollen), seeds, plant cells and the like. Plantlets and germinated seeds from which roots and shoots have emerged are also included within the meaning of “plant”. The term “plant parts” as used herein refers to one or more plant tissues or organs which are obtained from a plant and which comprises genomic DNA of the plant. Plant parts include vegetative structures (for example, leaves, stems), roots, floral organs/structures, seed (including embryo, cotyledons, and seed coat), plant tissue (for example, vascular tissue, ground tissue, and the like), cells and progeny of the same. The term “plant cell” as used herein refers to a cell obtained from a plant or in a plant and includes protoplasts or other cells derived from plants, gamete-producing cells, and cells which regenerate into whole plants. Plant cells may be cells in culture. By “plant tissue” is meant differentiated tissue in a plant or obtained from a plant (“explant”) or undifferentiated tissue derived from immature or mature embryos, seeds, roots, shoots, fruits, tubers, pollen, tumor tissue, such as crown galls, and various forms of aggregations of plant cells in culture, such as calli. Exemplary plant tissues in or from seeds are cotyledon, embryo and embryo axis. The invention accordingly includes plants and plant parts and products comprising these.

As used herein, the term “seed” refers to “mature seed” of a plant, which is either ready for harvesting or has been harvested from the plant, such as is typically harvested commercially in the field, or as “developing seed” which occurs in a plant after fertilisation and prior to seed dormancy being established and before harvest.

A “genetically modified plant”, or variations thereof, as used herein, refers to a plant that contains one or more genetic variations, such as introduced by gene editing, not found in a wild-type plant of the same species, variety or cultivar.

Plants contemplated for use in the practice of the present invention include both monocotyledons and dicotyledons. Target plants include, but are not limited to, the following: cereals (for example, wheat, barley, rye, oats, rice, maize, sorghum and related crops); grapes; beet (sugar beet and fodder beet); pomes, stone fruit and soft fruit (apples, pears, plums, peaches, almonds, cherries, strawberries, raspberries and black-berries); leguminous plants (beans, lentils, peas, soybeans); oil plants (rape or other Brassicas, mustard, poppy, olives, sunflowers, safflower, flax, coconut, castor oil plants, cocoa beans, groundnuts); cucumber plants (marrows, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocados, cinnamon, camphor); or plants such as maize, tobacco, nuts, coffee, sugar cane, tea, vines, hops, turf, bananas and natural rubber plants, as well as ornamentals (flowers, shrubs, broad-leaved trees and evergreens, such as conifers). Preferably, the plant is a cereal plant. In an embodiment, the cereal plant is a rice plant or sorghum plant. In an embodiment, the cereal plant is rice. In an embodiment, the cereal plant is maize. In an embodiment, the cereal plant is triticale. In an embodiment, the cereal plant is oats. In an embodiment, the cereal plant is barley.

In relation to dicotyledons and seed or bran therefrom, each of the embodiments relating to cereals, where relevant, also apply to dicotyledons of the invention.

As used herein, the term “rice” refers to any species of the Genus Oryza, including progenitors thereof, as well as progeny thereof produced by crosses with other species. It is preferred that the plant is of a Oryza species which is commercially cultivated such as, for example, a strain or cultivar or variety of Oryza sativa or suitable for commercial production of grain.

In an embodiment, the genetically modified plants are homozygous for each and every genetic variation that has been introduced so that their progeny do not segregate for the desired phenotype.

As used herein, the term “compared to an isogenic plant”, or similar phrases, refers to a plant which is isogenic, or is substantially isogenic relative to the genetically modified plant but without the genetic variations. Preferably, the corresponding isogenic plant is of the same cultivar or variety as the progenitor of the genetically modified plant of interest. “Wild type” or “corresponding”, as used herein, refers to a cell, tissue or plant that has not been modified according to the invention. Wild-type or corresponding cells, tissue or plants may be used as controls to compare levels of expression of mutant/variant proteins or the extent and nature of trait modification with cells, tissue or plants modified as described herein.

Genetically modified plants, as defined in the context of the present invention include progeny of the plants which have been genetically modified using recombinant techniques, wherein the progeny comprise the genetic variation of interest. Such progeny may be obtained by self-fertilisation of the primary genetically modified plant or by crossing such plants with another plant of the same species. This would generally be to modulate the production of at least one protein defined herein in the desired plant or plant organ. Genetically modified plant parts include all parts and cells of said plants comprising the genetic variations such as, for example, cultured tissues, callus and protoplasts.

Genetically modified plants, as defined in the context of the present invention include plants (as well as parts and cells of said plants) and their progeny which have been genetically modified using recombinant techniques to cause production of at least one polypeptide as defined herein in the desired plant or plant organ. Genetically modified plants can be produced using techniques known in the art, such as those generally described in A. Slater et al., Plant Biotechnology—The Genetic Manipulation of Plants, Oxford University Press (2003), N. G. Halford, Crop Technology: Genetic Modification and genome editing, World Scientific Publ Co Pte Ltd (2018), and P. Christou and H. Klee, Handbook of Plant Biotechnology, John Wiley and Sons (2004).

In an embodiment, the genetically modified plants are homozygous for each and every genetic modification that has been introduced so that their progeny do not segregate for the desired phenotype. The genetically modified plants may also be heterozygous for the introduced genetic modification(s), such as, for example, in F1 progeny which have been grown from hybrid seed. Such plants may provide advantages such as hybrid vigour, well known in the art.

Four general methods for direct delivery of a gene into cells have been described: (1) chemical methods (Graham et al., 1973); (2) physical methods such as microinjection (Capecchi, 1980); electroporation (see, for example, WO 87/06614, U.S. Pat. Nos. 5,472,869, 5,384,253, WO 92/09696 and WO 93/21335); and the gene gun (see, for example, U.S. Pat. Nos. 4,945,050 and 5,141,131); (3) viral vectors (Clapp, 1993; Lu et al., 1993; Eglitis et al., 1988); and (4) receptor-mediated mechanisms (Curiel et al., 1992; Wagner et al., 1992).

Acceleration methods that may be used include, for example, microprojectile bombardment and the like. One example of a method for delivering transforming nucleic acid molecules to plant cells is microprojectile bombardment. This method has been reviewed by Yang et al., Particle Bombardment Technology for Gene Transfer, Oxford Press, Oxford, England (1994). Non-biological particles (microprojectiles) that may be coated with nucleic acids and delivered into cells by a propelling force. Exemplary particles include those comprised of tungsten, gold, platinum, and the like. A particular advantage of microprojectile bombardment, in addition to it being an effective means of reproducibly transforming monocots, is that neither the isolation of protoplasts, nor the susceptibility of Agrobacterium infection are required. A particle delivery system suitable for use with the present invention is the helium acceleration PDS-1000/He gun is available from Bio-Rad Laboratories. For the bombardment, immature embryos or derived target cells such as scutella or calli from immature embryos may be arranged on solid culture medium.

In another alternative embodiment, plastids can be stably transformed. Method disclosed for plastid transformation in higher plants include particle gun delivery of DNA containing a selectable marker and targeting of the DNA to the plastid genome through homologous recombination (U.S. Pat. Nos. 5,451,513, 5,545,818, 5,877,402, 5,932,479, and WO 99/05265).

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art (see, for example, U.S. Pat. Nos. 5,177,010, 5,104,310, 5,004,863, 5,159,135). Further, the integration of the T-DNA is a relatively precise process resulting in few rearrangements. The region of DNA to be transferred is defined by the border sequences, and intervening DNA is usually inserted into the plant genome.

Agrobacterium transformation vectors are capable of replication in E. coli as well as Agrobacterium, allowing for convenient manipulations as described (Klee et al., Plant DNA Infectious Agents, Hohn and Schell, (editors), Springer-Verlag, New York, (1985): 179-203). Moreover, technological advances in vectors for Agrobacterium-mediated gene transfer have improved the arrangement of genes and restriction sites in the vectors to facilitate construction of vectors capable of expressing various polypeptide coding genes. The vectors described have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant varieties where Agrobacterium-mediated transformation is efficient, it is the method of choice because of the facile and defined nature of the gene transfer.

A genetically modified plant formed using Agrobacterium transformation methods typically contains a single genetic locus on one chromosome. Such genetically modified plants can be referred to as being hemizygous for the added gene. More preferred is a genetically modified plant that is homozygous for the added structural gene; i.e., a genetically modified plant that contains two added genes, one gene at the same locus on each chromosome of a chromosome pair. A homozygous genetically modified plant can be obtained by sexually mating (selfing) an independent segregant genetically modified plant that contains a single added gene, germinating some of the seed produced and analyzing the resulting plants for the gene of interest.

It is also to be understood that two different genetically modified plants can also be mated/crossed to produce offspring that contain two independently segregating exogenous genes. Selfing of appropriate progeny can produce plants that are homozygous for both exogenous genes. Back-crossing to a parental plant and out-crossing with a non-genetically modified plant are also contemplated, as is vegetative propagation. Descriptions of other breeding methods that are commonly used for different traits and crops can be found in Fehr, Breeding Methods for Cultivar Development, J. Wilcox (editor) American Society of Agronomy, Madison Wis. (1987).

Transformation of plant protoplasts can be achieved using methods based on calcium phosphate precipitation, polyethylene glycol treatment, electroporation, and combinations of these treatments. Application of these systems to different plant varieties depends upon the ability to regenerate that particular plant strain from protoplasts. Illustrative methods for the regeneration of cereals from protoplasts are described (Fujimura et al., 1985; Toriyama et al., 1986; Abdullah et al., 1986).

Other methods of cell transformation can also be used and include but are not limited to introduction of polynucleotides such as DNA into plants by direct transfer into pollen, by direct injection of polynucleotides such as DNA into reproductive organs of a plant, or by direct injection of polynucleotides such as DNA into the cells of immature embryos followed by the rehydration of desiccated embryos.

The regeneration, development, and cultivation of plants from single plant protoplast transformants or from various transformed explants is well known in the art (Weissbach et al., Methods for Plant Molecular Biology, Academic Press, San Diego, (1988)). This regeneration and growth process typically includes the steps of selection of transformed cells, culturing those individualized cells through the usual stages of embryonic development through the rooted plantlet stage. Genetically modified embryos and seeds are similarly regenerated. The resulting genetically modified rooted shoots are thereafter planted in an appropriate plant growth medium such as soil.

The development or regeneration of plants containing the foreign, exogenous gene is well known in the art. Preferably, the regenerated plants are self-pollinated to provide homozygous genetically modified plants. Otherwise, pollen obtained from the regenerated plants is crossed to seed-grown plants of agronomically important lines. Conversely, pollen from plants of these important lines is used to pollinate regenerated plants. A genetically modified plant of the present invention containing a desired genetic modification is cultivated using methods well known to one skilled in the art.

Methods for transforming dicots, primarily by use of Agrobacterium tumefaciens, and obtaining genetically modified plants have been published for cotton (U.S. Pat. Nos. 5,004,863, 5,159,135, 5,518,908); soybean (U.S. Pat. Nos. 5,569,834, 5,416,011); Brassica (U.S. Pat. No. 5,463,174); peanut (Cheng et al., 1996); and pea (Grant et al., 1995).

Methods for transformation of cereal plants such as wheat and barley for introducing genetic variation into the plant by introduction of an exogenous nucleic acid and for regeneration of plants from protoplasts or immature plant embryos are well known in the art, see for example, CA 2,092,588, AU 61781/94, AU 667939, U.S. Pat. No. 6,100,447, WO 97/048814, U.S. Pat. Nos. 5,589,617, 6,541,257, and other methods are set out in WO 99/14314. Preferably, genetically modified wheat or barley plants are produced by Agrobacterium tumefaciens mediated transformation procedures. Vectors carrying the desired nucleic acid construct may be introduced into regenerable wheat cells of tissue cultured plants or explants, or suitable plant systems such as protoplasts. The regenerable wheat cells are preferably from the scutellum of immature embryos, mature embryos, callus derived from these, or the meristematic tissue.

To confirm the presence of the genetic variation(s) in genetically modified cells and plants, a polymerase chain reaction (PCR) amplification or Southern blot analysis can be performed using methods known to those skilled in the art. Expression products of the genetically modified gene(s) can be detected in any of a variety of ways, depending upon the nature of the product, and include Western blot and enzyme assay. One particularly useful way to quantitate protein expression and to detect replication in different plant tissues is to use a reporter gene, such as GUS. Once genetically modified plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

### Marker Assisted Selection

Marker assisted selection is a well recognised method of selecting for heterozygous plants required when backcrossing with a recurrent parent in a classical breeding program. The population of plants in each backcross generation will be heterozygous for the gene(s) of interest normally present in a 1:1 ratio in a backcross population, and the molecular marker can be used to distinguish the two alleles of the gene. By extracting DNA from, for example, young shoots and testing with a specific marker for the introgressed desirable trait, early selection of plants for further backcrossing is made whilst energy and resources are concentrated on fewer plants. To further speed up the backcrossing program, the embryo from immature seeds (25 days post anthesis) may be excised and grown up on nutrient media under sterile conditions, rather than allowing full seed maturity.

Any molecular biological technique known in the art can be used in the methods of the present invention. Such methods include, but are not limited to, the use of nucleic acid amplification, nucleic acid sequencing, nucleic acid hybridization with suitably labelled probes, single-strand conformational analysis (SSCA), denaturing gradient gel electrophoresis (DGGE), heteroduplex analysis (HET), chemical cleavage analysis (CCM), catalytic nucleic acid cleavage or a combination thereof (see, for example, Lemieux, 2000; Langridge et al., 2001). The invention also includes the use of molecular marker techniques to detect polymorphisms linked to alleles of the (for example) FAD 2-1 gene or LOX3 gene conferring reduced activity. Such methods include the detection or analysis of restriction fragment length polymorphisms (RFLP), RAPD, amplified fragment length polymorphisms (AFLP) and microsatellite (simple sequence repeat, SSR) polymorphisms. The closely linked markers can be obtained readily by methods well known in the art, such as Bulked Segregant Analysis, as reviewed by Langridge et al. (2001).

In an embodiment, a linked loci for marker assisted selection is at least within 1 cM, or 0.5 cM, or 0.1 cM, or 0.01 cM from a gene encoding a polypeptide of the invention.

The “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme. Methods for PCR are known in the art, and are taught, for example, in “PCR” (M. J. McPherson and S. G Moller (editors), BIOS Scientific Publishers Ltd, Oxford, (2000)). PCR can be performed on cDNA obtained from reverse transcribing mRNA isolated from plant cells expressing a FAD 2-1 gene and/or LOX3 gene which confers upon the plant an altered grain fatty acid content. However, it will generally be easier if PCR is performed on genomic DNA isolated from a plant.

A primer is an oligonucleotide sequence that is capable of hybridising in a sequence specific fashion to the target sequence and being extended during the PCR. Amplicons or PCR products or PCR fragments or amplification products are extension products that comprise the primer and the newly synthesized copies of the target sequences. Multiplex PCR systems contain multiple sets of primers that result in simultaneous production of more than one amplicon. Primers may be perfectly matched to the target sequence or they may contain internal mismatched bases that can result in the introduction of restriction enzyme or catalytic nucleic acid recognition/cleavage sites in specific target sequences. Primers may also contain additional sequences and/or contain modified or labelled nucleotides to facilitate capture or detection of amplicons. Repeated cycles of heat denaturation of the DNA, annealing of primers to their complementary sequences and extension of the annealed primers with polymerase result in exponential amplification of the target sequence. The terms target or target sequence or template refer to nucleic acid sequences which are amplified.

Methods for direct sequencing of nucleotide sequences are well known to those skilled in the art and can be found for example in Ausubel et al., (supra) and Sambrook et al., (supra). Sequencing can be carried out by any suitable method, for example, dideoxy sequencing, chemical sequencing or variations thereof. Direct sequencing has the advantage of determining variation in any base pair of a particular sequence.

### Tilling

Plants of the invention can be produced using the process known as TILLING (Targeting Induced Local Lesions IN Genomes). In a first step, introduced mutations such as novel single base pair changes are induced in a population of plants by treating seeds (or pollen) with a chemical mutagen, and then advancing plants to a generation where mutations will be stably inherited. DNA is extracted, and seeds are stored from all members of the population to create a resource that can be accessed repeatedly over time.

For a TILLING assay, PCR primers are designed to specifically amplify a single gene target of interest. Specificity is especially important if a target is a member of a gene family or part of a polyploid genome. Next, dye-labeled primers can be used to amplify PCR products from pooled DNA of multiple individuals. These PCR products are denatured and reannealed to allow the formation of mismatched base pairs. Mismatches, or heteroduplexes, represent both naturally occurring single nucleotide polymorphisms (SNPs) (i.e., several plants from the population are likely to carry the same polymorphism) and induced SNPs (i.e., only rare individual plants are likely to display the mutation). After heteroduplex formation, the use of an endonuclease, such as Cel I, that recognizes and cleaves mismatched DNA is the key to discovering novel SNPs within a TILLING population.

Using this approach, many thousands of plants can be screened to identify any individual with a single base change as well as small insertions or deletions (1-30 bp) in any gene or specific region of the genome. Genomic fragments being assayed can range in size anywhere from 0.3 to 1.6 kb. At 8-fold pooling, 1.4 kb fragments (discounting the ends of fragments where SNP detection is problematic due to noise) and 96 lanes per assay, this combination allows up to a million base pairs of genomic DNA to be screened per single assay, making TILLING a high-throughput technique.

TILLING is further described in Slade and Knauf (2005), and Henikoff et al. (2004).

In addition to allowing efficient detection of mutations, high-throughput TILLING technology is ideal for the detection of natural polymorphisms. Therefore, interrogating an unknown homologous DNA by heteroduplexing to a known sequence reveals the number and position of polymorphic sites. Both nucleotide changes and small insertions and deletions are identified, including at least some repeat number polymorphisms. This has been called Ecotilling (Comai et al., 2004).

Each SNP is recorded by its approximate position within a few nucleotides. Thus, each haplotype can be archived based on its mobility. Sequence data can be obtained with a relatively small incremental effort using aliquots of the same amplified DNA that is used for the mismatch-cleavage assay. The left or right sequencing primer for a single reaction is chosen by its proximity to the polymorphism. Sequencher software performs a multiple alignment and discovers the base change, which in each case confirmed the gel band.

Ecotilling can be performed more cheaply than full sequencing, the method currently used for most SNP discovery. Plates containing arrayed ecotypic DNA can be screened rather than pools of DNA from mutagenized plants. Because detection is on gels with nearly base pair resolution and background patterns are uniform across lanes, bands that are of identical size can be matched, thus discovering and genotyping SNPs in a single step. In this way, ultimate sequencing of the SNP is simple and efficient, made more so by the fact that the aliquots of the same PCR products used for screening can be subjected to DNA sequencing.

### Plant/Grain Processing

Grain/seed of the invention, preferably cereal grain and more preferably rice or sorghum grain, or other plant parts of the invention, can be processed to produce a food ingredient, food or non-food product using any technique known in the art.

Techniques that are routinely practiced in the art can be used to extract, process, and analyze the oils produced by cells, plants, seeds, bran etc of the instant invention. Typically, plant seeds are cooked, pressed, and extracted to produce crude oil, which is then degummed, refined, bleached, and deodorized. Rice is typically milled to remove the husk and polished to remove the bran layer from the white rice. Generally, techniques for crushing seed and bran are known in the art. For example, seeds can be tempered by spraying them with water to raise the moisture content to, e.g., 8.5%, and flaked using a smooth roller with a gap setting of 0.23 to 0.27 mm. Depending on the type of seed, water may not be added prior to crushing. Rice bran may be heated by steam at or above 100° C. Application of heat deactivates enzymes, facilitates further cell rupturing, coalesces the oil droplets, and agglomerates protein particles, all of which facilitate the extraction process.

Rice bran is separated during rice milling. The bran can be stabilised usually by applying heat or irradiation and then the rice bran oil recovered using chemical and/or physical methods as described. For a review of rice bran and rice bran oil see Rice Bran and Rice Bran Oil Chemistry, Processing and Utilization AOCS Press, (2019) Editor(s): Ling-Zhi Cheong, Xuebing Xu, ISBN 9780128128282. The defatted rice bran provides a nutrient rich meal that is suitable for human food and animal feed. Further processing to isolate valuable fatty acids, starch or phytates from the rice bran oil or meal can be performed. Alternatively, the rice bran may be fermented.

In an embodiment, the majority of the oil is released by passage through a screw press. Cakes expelled from the screw press are then solvent extracted, e.g., with hexane, using a heat traced column. Alternatively, crude oil produced by the pressing operation can be passed through a settling tank with a slotted wire drainage top to remove the solids that are expressed with the oil during the pressing operation. The clarified oil can be passed through a plate and frame filter to remove any remaining fine solid particles. If desired, the oil recovered from the extraction process can be combined with the clarified oil to produce a blended crude oil.

Once the solvent is stripped from the crude oil, the pressed and extracted portions are combined and subjected to normal oil processing procedures. As used herein, the term “purified” when used in connection with oil of the invention typically means that that the extracted lipid or oil has been subjected to one or more processing steps of increase the purity of the lipid/oil component. For example, a purification step may comprise one or more or all of the group consisting of degumming, deodorising, decolourising, drying and/or fractionating the extracted oil.

Degumming is an early step in the refining of oils and its primary purpose is the removal of most of the phospholipids from the oil, which may be present as approximately 1-2% of the total extracted lipid. Addition of ˜2% of water, typically containing phosphoric acid, at 70-80° C. to the crude oil results in the separation of most of the phospholipids accompanied by trace metals and pigments. The insoluble material that is removed is mainly a mixture of phospholipids and triacylglycerols and is also known as lecithin. Degumming can be performed by addition of concentrated phosphoric acid to the crude seedoil to convert non-hydratable phosphatides to a hydratable form, and to chelate minor metals that are present. Gum is separated from the soil by centrifugation.

Alkali refining is one of the refining processes for treating crude oil, sometimes also referred to as neutralization. It usually follows degumming and precedes bleaching. Following degumming, the oil can treated by the addition of a sufficient amount of an alkali solution to titrate all of the fatty acids and phosphoric acids, and removing the soaps thus formed. Suitable alkaline materials include sodium hydroxide, potassium hydroxide, sodium carbonate, lithium hydroxide, calcium hydroxide, calcium carbonate and ammonium hydroxide. This process is typically carried out at room temperature and removes the free fatty acid fraction. Soap is removed by centrifugation or by extraction into a solvent for the soap, and the neutralised oil is washed with water. If required, any excess alkali in the oil may be neutralized with a suitable acid such as hydrochloric acid or sulphuric acid.

Bleaching is a refining process in which oils are heated at 90-120° C. for 10-30 minutes in the presence of a bleaching earth (0.2-2.0%) and in the absence of oxygen by operating with nitrogen or steam or in a vacuum. This step in oil processing is designed to remove unwanted pigments (carotenoids, chlorophyll, gossypol etc), and the process also removes oxidation products, trace metals, sulphur compounds and traces of soap.

Deodorization is a treatment of oils and fats at a high temperature (200-260° C.) and low pressure (0.1-1 mm Hg). This is typically achieved by introducing steam into the oil at a rate of about 0.1 ml/minute/100 ml of oil. After about 30 minutes of sparging, the oil is allowed to cool under vacuum. The oil is typically transferred to a glass container and flushed with argon before being stored under refrigeration. This treatment improves the colour of the oil and removes a majority of the volatile substances or odorous compounds including any remaining free fatty acids, monoacylglycerols and oxidation products.

Winterization is a process sometimes used in commercial production of oils for the separation of oils and fats into solid (stearin) and liquid (olein) fractions by crystallization at sub-ambient temperatures. It was applied originally to cottonseed oil to produce a solid-free product. It is typically used to decrease the saturated fatty acid content of oils.

Transesterification is a process that exchanges the fatty acids within and between TAGs or transfers the fatty acids to another alcohol to form an ester, initially by releasing fatty acids from the TAGs either as free fatty acids or as fatty acid esters, usually fatty acid methyl esters or ethyl esters. When combined with a fractionation process, transesterification can be used to modify the fatty acid composition of lipids. Transesterification can use either chemical (e.g. strong acid or base catalysed) or enzymatic means, the latter using lipases which may be position-specific (sn-⅓ or sn-2 specific) for the fatty acid on the TAG, or having a preference for some fatty acids over others. The fatty acid fractionation to increase the concentration of LC-PUFA in an oil can be achieved by any of the methods known in the art, such as, for example, freezing crystallization, complex formation using urea, molecular distillation, supercritical fluid extraction and silver ion complexing. Complex formation with urea is a preferred method for its simplicity and efficiency in reducing the level of saturated and monounsaturated fatty acids in the oil. Initially, the TAGs of the oil are split into their constituent fatty acids, often in the form of fatty acid esters, by hydrolysis under either acid or base catalysed reaction conditions, whereby one mol of TAG is reacted with at least 3 mol of alcohol (e.g. ethanol for ethyl esters or methanol for methyl esters) with excess alcohol used to enable separation of the formed alkyl esters and the glycerol that is also formed, or by lipases. These free fatty acids or fatty acid esters, which are usually unaltered in fatty acid composition by the treatment, may then be mixed with an ethanolic solution of urea for complex formation.

In one embodiment, the product is whole grain flour such as, for example, an ultrafine-milled whole grain flour, or a flour made from about 100% of the grain. The whole grain flour includes a refined flour constituent (refined flour or refined flour) and a coarse fraction (an ultrafine-milled coarse fraction).

Refined flour may be flour which is prepared, for example, by grinding and bolting cleaned grain such as rice or sorghum grain. The particle size of refined flour is described as flour in which not less than 98% passes through a cloth having openings not larger than those of woven wire cloth designated “212 micrometers (U.S. Wire 70)”. The coarse fraction includes at least one of bran and germ. For instance, the germ is an embryonic plant found within the grain kernel. The germ includes lipids, fiber, vitamins, protein, minerals and phytonutrients, such as flavonoids. The bran includes several cell layers and has a significant amount of lipids, fiber, vitamins, protein, minerals and phytonutrients, such as flavonoids. Further, the coarse fraction may include an aleurone layer which also includes lipids, fiber, vitamins, protein, minerals and phytonutrients, such as flavonoids. The aleurone layer, while technically considered part of the endosperm, exhibits many of the same characteristics as the bran and therefore is typically removed with the bran and germ during the milling process. The aleurone layer contains proteins, vitamins and phytonutrients, such as ferulic acid.

Further, the coarse fraction may be blended with the refined flour constituent. The coarse fraction may be mixed with the refined flour constituent to form the whole grain flour, thus providing a whole grain flour with increased nutritional value, fiber content, and antioxidant capacity as compared to refined flour. For example, the coarse fraction or whole grain flour may be used in various amounts to replace refined or whole grain flour in baked goods, snack products, and food products. The whole grain flour of the present invention (i.e.—ultrafine-milled whole grain flour) may also be marketed directly to consumers for use in their homemade baked products. In an exemplary embodiment, a granulation profile of the whole grain flour is such that 98% of particles by weight of the whole grain flour are less than 212 micrometers.

In further embodiments, enzymes found within the bran and germ of the whole grain flour and/or coarse fraction are inactivated in order to stabilize the whole grain flour and/or coarse fraction. Stabilization is a process that uses steam, heat, radiation, or other treatments to inactivate the enzymes found in the bran and germ layer. Flour that has been stabilized retains its cooking characteristics and has a longer shelf life.

In additional embodiments, the whole grain flour, the coarse fraction, or the refined flour may be a component (ingredient) of a food product and may be used to product a food product. For example, the food product may be a bagel, a biscuit, a bread, a bun, a croissant, a dumpling, an English muffin, a muffin, a pita bread, a quickbread, a refrigerated/frozen dough product, dough, baked beans, a burrito, chili, a taco, a tamale, a tortilla, a pot pie, a ready to eat cereal, a ready to eat meal, stuffing, a microwaveable meal, a brownie, a cake, a cheesecake, a coffee cake, a cookie, a dessert, a pastry, a sweet roll, a candy bar, a pie crust, pie filling, baby food, a baking mix, a batter, a breading, a gravy mix, a meat extender, a meat substitute, a seasoning mix, a soup mix, a gravy, a roux, a salad dressing, a soup, sour cream, a noodle, a pasta, ramen noodles, chow mein noodles, lo mein noodles, an ice cream inclusion, an ice cream bar, an ice cream cone, an ice cream sandwich, a cracker, a crouton, a doughnut, an egg roll, an extruded snack, a fruit and grain bar, a microwaveable snack product, a nutritional bar, a pancake, a par-baked bakery product, a pretzel, a pudding, a granola-based product, a snack chip, a snack food, a snack mix, a waffle, a pizza crust, animal food or pet food.

In alternative embodiments, the whole grain flour, refined flour, or coarse fraction may be a component of a nutritional supplement. For instance, the nutritional supplement may be a product that is added to the diet containing one or more additional ingredients, typically including: vitamins, minerals, herbs, amino acids, enzymes, antioxidants, herbs, spices, probiotics, extracts, prebiotics and fiber. The whole grain flour, refined flour or coarse fraction of the present invention includes vitamins, minerals, amino acids, enzymes, and fiber. For instance, the coarse fraction contains a concentrated amount of dietary fiber as well as other essential nutrients, such as B-vitamins, selenium, chromium, manganese, magnesium, and antioxidants, which are essential for a healthy diet. For example 22 grams of the coarse fraction of the present invention delivers 33% of an individual's daily recommend consumption of fiber. The nutritional supplement may include any known nutritional ingredients that will aid in the overall health of an individual, examples include but are not limited to vitamins, minerals, other fiber components, fatty acids, antioxidants, amino acids, peptides, proteins, lutein, ribose, omega-3 fatty acids, and/or other nutritional ingredients. The supplement may be delivered in, but is not limited to the following forms: instant beverage mixes, ready-to-drink beverages, nutritional bars, wafers, cookies, crackers, gel shots, capsules, chews, chewable tablets, and pills. One embodiment delivers the fiber supplement in the form of a flavored shake or malt type beverage, this embodiment may be particularly attractive as a fiber supplement for children.

In an additional embodiment, a milling process may be used to make a multi-grain flour or a multi-grain coarse fraction. For example, bran and germ from one type of grain may be ground and blended with ground endosperm or whole grain cereal flour of another type of cereal. Alternatively, bran and germ of one type of grain may be ground and blended with ground endosperm or whole grain flour of another type of grain. It is contemplated that the present invention encompasses mixing any combination of one or more of bran, germ, endosperm, and whole grain flour of one or more grains. This multi-grain approach may be used to make custom flour and capitalize on the qualities and nutritional contents of multiple types of cereal grains to make one flour.

It is contemplated that the whole grain flour, coarse fraction and/or grain products of the present invention may be produced by any milling process known in the art. An exemplary embodiment involves grinding grain in a single stream without separating endosperm, bran, and germ of the grain into separate streams. Clean and tempered grain is conveyed to a first passage grinder, such as a hammermill, roller mill, pin mill, impact mill, disc mill, air attrition mill, gap mill, or the like. After grinding, the grain is discharged and conveyed to a sifter. Further, it is contemplated that the whole grain flour, coarse fraction and/or grain products of the present invention may be modified or enhanced by way of numerous other processes such as: fermentation, instantizing, extrusion, encapsulation, toasting, roasting, or the like.

### Malting

A malt-based beverage provided by the present invention involves alcohol beverages (including distilled beverages) and non-alcohol beverages that are produced by using malt as a part or whole of their starting material. Examples include beer, happoshu (low-malt beer beverage), whisky, low-alcohol malt-based beverages (e.g., malt-based beverages containing less than 1% of alcohols), and non-alcohol beverages.

Malting is a process of controlled steeping and germination followed by drying of the grain such as barley and wheat grain. This sequence of events is important for the synthesis of numerous enzymes that cause grain modification, a process that principally depolymerizes the dead endosperm cell walls and mobilizes the grain nutrients. In the subsequent drying process, flavour and colour are produced due to chemical browning reactions. Although the primary use of malt is for beverage production, it can also be utilized in other industrial processes, for example as an enzyme source in the baking industry, or as a flavouring and colouring agent in the food industry, for example as malt or as a malt flour, or indirectly as a malt syrup, etc.

In one embodiment, the present invention relates to methods of producing a malt composition. The method preferably comprises the steps of:
(i) providing grain, such as barley or wheat grain, of the invention,
(ii) steeping said grain,
(iii) germinating the steeped grains under predetermined conditions and
(iv) drying said germinated grains.

For example, the malt may be produced by any of the methods described in Hoseney (Principles of Cereal Science and Technology, Second Edition, 1994: American Association of Cereal Chemists, St. Paul, Minn.). However, any other suitable method for producing malt may also be used with the present invention, such as methods for production of specialty malts, including, but limited to, methods of roasting the malt.

Malt is mainly used for brewing beer, but also for the production of distilled spirits. Brewing comprises wort production, main and secondary fermentations and post-treatment. First the malt is milled, stirred into water and heated. During this “mashing”, the enzymes activated in the malting degrade the starch of the kernel into fermentable sugars. The produced wort is clarified, yeast is added, the mixture is fermented and a post-treatment is performed.

### EXAMPLES

### Example 1—Materials and Methods

### Generation of Mutants

gRNA Design

The in silico analysis of DNA sequences of rice FAD2, LOX3 and FATBs, DNA sequence annotation and alignment, vector designs and protein sequence prediction were performed using Geneius Prime 2019.1.1 (www.geneious.com). Rice OsFAD2-1, OsLOX3 and OsFATB1, 2, 3, 4 gene sequences were obtained from Rice Genome Annotation Project (rice.plantbiology.msu.edu/) for designing the gRNAs. All the gRNAs were GC rich, 19 or 20 bn in length, and linked to the canonical PAM (5′-NGG-3′). The gene edit vector V1 targeted OsFAD2-1, OsLOX3 and OsFATB1, while the gene edit vector V2 targeted OsFATB1, 2, 3 and 4. In vector V1, gRNA-1 was targeted for nt 3317˜3336 of LOC_OS02g48560 (OsFAD2-1) corresponding to nt 3˜22 (20 bp) of CDS, covering the second in-frame ATG (nt 19˜21 of CDS). gRNA-2 was targeted for nt 3846˜3865 of LOC_OS02g48560, corresponding to nt 532˜551 of OsFAD2-1 CDS. The gRNA-3 was designed at the #31 to #50 region in the 1^{st} exon, and the gRNA-4 at the 20 bp between #467 and #486 crossing 1st intron and 2^{nd} exon of OsLOX3. The gRNA-5 was designed at the beginning of 2^{nd} exon (#523-542) of OsFATB1. In the V2, gRNA-6 targeted #323-342 in the 1^{st} exon of OsFATB1. gRNA-7 was designed to the conserved region at #451-470 in the second exon of both OsFATB2 and 3. gRNA-8 targeted 20 bp between #624-643 of the 2^{nd} exon of OsFATB4.

### Vector Construction

The multi-gRNA expression cassettes consisting of the two OsU3 promoters and three OsU6 promoters (OsU6a, OsU6b, OsU6c) driving gRNA-1-5, respectively, were commercially synthesized at GeneArt (Thermo Fisher Scientific, Regensburg, Germany). Each cassette was cloned into BsaI restriction site of pMA-RQ by GeneArt. The expression cassettes in pMA-RQ were then cloned into pYLCRISPR/Cas9Pubi-H by simultaneous digestion with BsaI and ligating with Golden Gate cloning system following the description by Ma et al. (2015).

In a 15 μL reaction, 50 ng of each gRNA expression cassettes and vector were mixed with BsaI, T4 ligase and ligation buffer. The ligation reaction was set up in the PCR machine with 25 cycles of 37° C., 3 min and 16° C., 4 min, and 1 cycle of 50° C., 5 min and 80° C., 5 min. Then the product was held at 4° C. before transforming into Agrobacterium. The resulting positive clones (V1 and V2, see Example 2 for details of clones) were confirmed by amplifying plasmid DNA fragment containing the gRNA cassettes using PCR markers (primers SP1 and SP2). The molecular sizes of PCR products of V1 and V2 were 3143 bp and 1739 bp, respectively. The V1 and V2 vectors were used to transform rice by Agrobacterium-mediated transformation.

### Agrobacterium-Mediated Transformation of Rice

The vectors V1 or V2 were transformed into Agrobacterium strain AGL1 for rice transformation (Toki et al., 2006) with slight modification. The husk was removed from mature seeds of Nipponbare cv. The seeds were then sterilized with 75% ethanol and 25% bleach consecutively and followed by 8 times of wash in MilliQ water. The seeds were transferred to N6D solid medium pH 5.8 (CHU [N6] Basal Medium with vitamins 3.99 g/L, Myoinositol 100 mg/L, Peptone (crude protein) 300 mg/L, Proline 2.9 g/L, Sucrose 30 g/L, and 2 m/L of 2,4-D (1 mg/ml)) to induce callus in a 25° C. growth room under 24 hrs purple and blue light.

2N6-AS medium*

| Chemicals | Amount for 1 L | |
|---|---|---|
| CHU [N6] Basal Medium with vitamins | 3.99 | g |
| Myoinositol | 100 | mg |
| Casamino acids | 300 | mg |
| Glucose | 10 | g |
| Sucrose | 30 | g |
| 2,4-D (1 mg/mL) | 2 | mL |
| Dissolve 100 mg of 2,4-D in 1 mL ethanol | | |
| Add 3 ml of 1N KOH | | |
| Adjust to H 6 with 1N HCl | | |

*Adjust pH to 5.2 with 1M KOH/NaOH. Add acetosyringone 2 mg/L when use. Autoclave.

Regeneration Medium*

| Chemicals | Amount for 1 L | |
|---|---|---|
| Sucrose | 30 | g |
| Sorbitol | 30 | g |
| Casamino acids | 2 | g |
| MS-basal salts or with vitamins | 4.33 | g |
| B5 vitamins (×1000) - optional for | 1 | ml |
| MS-basal without vitamins | | |
| Myoinositol | 10 | g |
| Nicotinic acid | 100 | mg |
| Pyridoxine HCl | 100 | mg |
| Thiamin HCl | 1 | g |
| MiliQ water | 100 | ml |
| NAA (stock 2 mg/ml) | 10 | μl |
| Kinetin (stock 5 mg/ml) | 400 | μl |

*Adjust pH to 5.8 with 1M KOH/NaOH. Add phytogel 3 g/L. Autoclave.

Calli were collected after 4-6 weeks of induction and co-cultivated with the AGL1 harboring V1 or V2 in 2N6-AS liquid medium for 3 days at room temperature in the dark. The calli were then washed with sterile water containing timentin (150 mg/L) and blotted dry with filter paper. The transformed calli were selected on N6D medium plate containing timentin (150 mg/L) and antibiotic hygromycin (35 mg/L) at room temperature in the dark for 3-4 weeks. The resistant calli were transferred to the regeneration medium containing hygromycin to develop root and incubated at room temperature in the growth room in the dark for 3-4 weeks. The regenerated plantlets were transferred to MS medium containing timentin and hygromycin for further plant development in the growth room.

MS medium*

| Chemicals | Amount for 1 L | |
|---|---|---|
| Sucrose | 30 | g |
| MS-basal salts or with vitamins | 4.33 | g |
| B5 vitamins (×1000) - optional for | 1 | mL |
| MS-basal without vitamins | | |
| Myoinositol | 10 | g |
| Nicotinic acid | 100 | mg |
| Pyridoxine HCl | 100 | mg |
| Thiamin HCl | 1 | g |
| MiliQ water | 100 | mL |

*Adjust pH to 5.8 with 1M KOH/NaOH. Add phytogel 3 g/L. Autoclave.

The transformed rice plants were then transplanted into pots with general purpose potting mix and held in the growth chamber (12 hours photoperiod, 30° C. in the light and 24° C. in the dark). The plants were watered every three (3) days until tiller stage when the potted plants were transferred to the glass house.

### Determination of Targeted Mutations in the Rice Plants

T₀ to T₃ plants of 14 transgenic lines were grown in the phytotron at CSIRO Black Mountain Scientific Innovation Park (Canberra, ACT, Australia) at 22° C. to 26.5° C. under natural daylight. Genomic DNA samples (100 ng/μL) were prepared from leaf tissue of each plant. Leaf tissue was quickly frozen in liquid nitrogen and ground using chopsticks in the 2 mL tubes. About 600 μL of extraction buffer (100 mM Tris-HCl pH 8.0, 50 mM EDTA, 1.25% SDS) were added for each sample to suspend by shaking. The samples were incubated at 65° C. in an oven for at least one hour. After cooled down, 300 μL of cold 6M ammonium acetate was added to each sample to precipitate at 4° C. Supernatant was collected by centrifuging and 300 μL isopropanol was added to precipitate the DNA. Pellet was collected by centrifuging and washed with 250 μL of 70% ethanol. About 50 μL Mili-Q water was added and the sample was kept at room temperature overnight to dissolve the DNA. The concentration of DNA was measured using NanoDrop Spectrophotometer (Model ND-1000, NanoDrop).

PCR primers were designed using Geneius Prime 2019.1.1 to amplify DNA fragments covering the gRNA targeted regions. The PCR mix contained 2 μL of 5× Taq polymerase buffer, 0.5 μL of each primer (10 nM), 100 ng template DNA and 0.07 μL of MyTaq polymerase (Thermo) to a final volume of 10 μL. The following PCR program was used for the amplification: 2 min at 95° C., 34 cycles of 15 sec at 95° C., 15 sec at 58° C. and 32 sec at 72° C., followed by 5 min at 72° C. and held at 12° C. The PCR products were diluted 10 times and cleaned up with Shrimp Alkaline Phosphatase (SAP) at ratio of 2.5:1 (v/v) for 15 min at 37° C. and 15 min at 80° C. The cleaned DNA product (2.8 μL) was mixed with 0.3 μL forward or reverse primer and BigDye buffer to a final volume of 20 μl. The PCR was carried out under following condition: 5 min at 94° C., 30 cycles of 10 sec at 96° C., 5 sec at 50° C. and 4 min at 60° C. and held at 12° C. The BigDye product was mixed with 2 μL of 3 M NaOAc (pH 4.8-5) and 50 μL of ice cold 100% Ethanol to precipitate at −80° C. for 30 min. The precipitate was washed with 200 μL of 70% ethanol and dried in a vacuum spin dryer before submitting for Sanger sequencing service (Australian National University (ANU) facility, Canberra Australia).

### Preparation of Fatty Acid Methyl Ester

After maturing, seeds were harvested and dried at 37° C. for about two weeks, then manually threshed. The single/multiple grains (single/half grain for screening, multiple grains for characterising phenotype of the homozygotes) of brown rice were then ground into flour using a 3M ESPE CapMax™ homogenizer (ESPE, Seefeld, Germany). About 3-5 mg of flour samples from the single/multiple grains was used for preparing fatty acid methyl ester (FAME) following the procedure described by Zaplin et al. (2013).

For leaf, root and anther tissues, the plant parts were dried in a freeze-drier (BencheTop Pro Model BTP-8ZLEVX, SP Scientific). Dried plant parts were sectioned by scissors and samples of 5-10 mg were weighed into 2-mL vials for each replicate. To each vial 600 μL of TN Methanolic HCl was added and samples were methylated at 80° C. for 2 hr with caps tightly closed. After the vials cooled down to room temperature, 300 μL of 0.9% NaCl and 300 μL hexane were added and mixed for 5 min in a shaker (Ratek Model MTV1, Ratek Instruments Pty Ltd, Australia). The samples were centrifuged at 1700 g for 5 min (Model 2-6E, Sigma). Approximately 280 μL of the upper hexane phase containing FAME was transferred to a conical glass insert and evaporated under nitrogen for 5-10 min. The FAME was re-dissolved in 50 μL hexane.

### Analysis of Fatty Acid Profile by Gas Chromatography

FAMEs were analysed by gas chromatography (7890A GC; Agilent Technologies, Santa Clara, CA) fitted with an SGE BPX70 column (0.25 mm diameter, 30 m length, 2.5 μm film thickness, Agilent) with 50:1 split, essentially as described (Zhou et al., 2011). The column temperature was programmed as an initial temperature at 150° C. held for 1 min, which was increased to 210° C. at 3° C./min, then further increased to 240° C. at 50° C./min which was held for 1.4 min. Helium was the carrier gas with a column head pressure of 17.334 psi and average velocity of 30 cm/sec. The fatty acid profile was analysed by integrating peaks with Agilent Technologies ChemStation software (Rev B.04.03), and the total fatty acid composition was calculated as percentage of whole in each sample.

### SPME GC-MS Analysis of Rice Bran Volatile Compounds

Rice bran (10% by whole grain weight) was collected from the TP-3000 PEARLEST Grain Polisher (Kett, USA) during polishing the brown rice grains. The bran samples were immediately stored at −80° C. to avoid staling. Volatile compounds were analysed headspace (HS)-SPME GC-MS using a 50/30 μm divinylbenzene-carboxen-polydimethylsiloxane (DVB/CAR/PDMS) Stableflex fiber (Supelco, USA) 10 mm long, for automatic autosamplers. Fibers were pre-conditioned at 270° C. for 30 min before use.

For the analysis of samples, 10 mL headspace magnetic cap vials containing the 100 mg of the samples were pre-incubated for 5 min at 60° C. prior extraction. Volatiles were extracted for 60 min at 60° C. under agitation using a Combi-Pal autosampler HTX PAL (CTC Analytics). Samples were desorbed at the injector temperature of 250° C. for 1 min at splitless mode. Fibre was conditioned in a needle heater with a helium flow for 5 min at 250° C. before and after sample desorption to reduce sample carryover. The volatile compounds desorbed from the fibre were analysed by a Shimadzu QP2010 Plus GC-MS equipped with a Shimadzu Stabilwax-DA column (30 m×0.25 mm×0.25 μm). The carrier gas was helium at a constant flow rate of 1 mL/min. Oven ramping program started at 45° C. held for 5.5 min, heated to 170° C. at a rate of 3° C./min and ramping at 7° C./min to a final temp of 250° C., being held for 2 min. Ion fragmentation was acquired under EI mode at 70 eV and scanned in full scan mode from 35 to 350 m/z. Volatiles were identified by comparing NIST mass spectra library and linear retention indexes calibration standards (even n-alkanes C10-C40). Purchased authentic standards from different compound classes and blanks (empty HS vials, with 10 ng of the internal standard 2,4,6-trimethylpyridine) were also analysed for analytical quality control. Mass spectra matches were only considered with a minimum of 80% similarity index.

### Example 2—Identification and Isolation of Fad2 Genes from Rice

Δ12-desaturases encoded by OsFAD2 genes (Fatty Acid Desaturase 2) are responsible for the introduction of a double bond into 18:1 fatty acid at Δ12 position. There were four members in the OsFAD2 gene family in rice (Zaplin et al., 2013), designated OsFAD2-1 (LOC_Os02g48560), OsFAD2-2 (LOC_Os07g23430), OsFAD2-3 (LOC_Os07g23410) and OsFAD2-4 (LOC_Os07g23390). The genomic sequences for these Oryza sativa FAD2 were retrieved from Genbank. The cDNA sequences were derived and shown in FIG. 1 which shows the alignment of Fad2 cDNA sequences. The OsFAD2-1 isoform has an in-frame ATG codon (nt 19˜21) after the translation start codon ATG. gRNAs designed for CRISPR editing of OsFAD2-1 included this second ATG (FIG. 1) as outlined below.

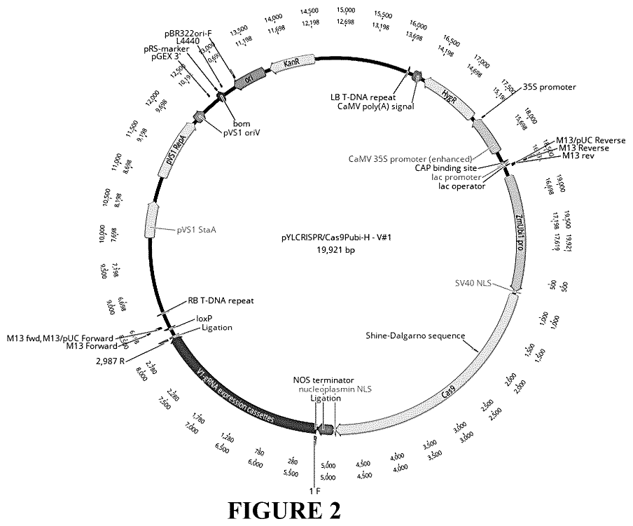

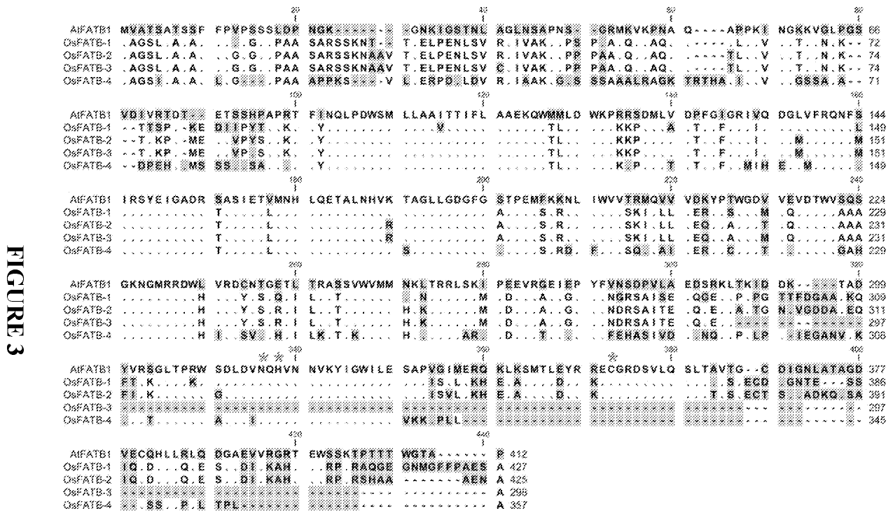

A schematic of the T-DNA binary vector used for transformation is shown in FIG. 2. The location of the stop codon is indicated by an asterisk in the modified FAD2 amino acid sequence for line V1-13 in Table 3.

gRNA and Vector Design of V1 and V2

For CRISPR-Cas9 editing the base pairing of the gRNA or single guide RNA controls how specific the cleavage is and the site of the cleavage. Guide RNA design was undertaken as described in Example 1. To evaluate the contribution of FAD2-1 gene to oil content in the grain, gRNA-1 (GGGTGCCGGCGGCAGGATGA) (SEQ ID NO:30) targeting position nt3-22 and gRNA-2 (TACGTGTACCACAACCCGAT) (SEQ ID NO:31) targeting position nt 532-551 were designed. FAD2-1 shares low homology with the three other members within the two gRNA regions. The gRNA-1 targeted the 20 bp from the end of start codon covering the second ATG, and the gRNA-2 at 532 bp downstream of the start codon in the FAD2-1 and used in the vector design of V1 in Example 5 and vector design V2 in Example 6.

### Example 3—Identification and Isolation of Lox3 Genes from Rice

Rice lipoxygenases (LOXs; EC 1.13.11.12) catalyze peroxidation of lipids. According to the protein sequences, LOXs are classified into three types (Mizuno et al., 2003). Type I lipoxygenase is localized in chloroplast and stress inducible; Type II lipoxygenase is localized in cytoplasm, derived from dicots, and is not stress inducible; Type III lipoxygenase is localized in the cytoplasm, derived from monocots and related to seed germination. Type I LOXs have a transit peptide, this is absent in Type II and Type III LOXs. LOXs are also classified as either 9-LOXs or 13-LOXs according to the enzymes preference for carbon 9 or carbon 13 in the substrate hydrocarbon backbone, generating 9(S)-hydroperoxy- and 9(S)-hydroperoxy-derivatives (Feussner and Wasternack, 2002). Based on bioinformatic analysis, it is purported that the rice genome (rice.plantbiology.msu.edu) has 14 LOX protein genes. Protein alignment shows that LOX sequences are relatively well conserved (Umate, 2011).

Polyunsaturated fatty acids (PUFAs) including linoleic and linolenic acids are common substrates in seeds for different LOXs. LOXs present in the rice grain are thought to play important roles in fatty acid peroxidation in membranes or storage lipids. LOX activity in rice seeds is associated with the production of volatile compounds such as n-hexanal, derived from lipid peroxidation, a predominant component of stale or rancid off-flavours in stored rice seed.

Three isozymes of Type III LOXs (LOX1, LOX2, and LOX3) have been identified in developing rice seeds (Ohta et al., 1986). Among them, LOX3 is the most abundant enzyme (Ida et al., 1983). The LOX3 enzyme was purified and characterized as a 9-LOX (Ohta et al., 1986). The Thai rice variety, DawDam contains a point mutation that causes the premature stop of translation of the LOX3 gene, resulting in a null-mutant rice variety (Suzuki et al., 1993; Suzuki and Matsukura, 1997). The mutation has been associated with reduction in stale flavor development during rice grain storage (Suzuki et al., 1999). Xu et al. (2015) used RNAi silencing to reduce the LOX3 activity in transgenic experimental plants resulted in grain with improved seed storability. Conversely, Ma et al. (2015) used TALENs to target and downregulate LOX3 and confirmed seed aging is a complex process. They proposed that LOX3 seed longevity effects may be unrelated to the peroxidation of fatty acids due to the purported functional redundancy between the LOX1, LOX2 and LOX3 isoenzymes in the rice grain. Although LOX3 deficiency is not shown to effect the main agronomic traits in rice (Ma et al., 2015) it is recognised that silencing all of LOX1, LOX2 and LOX3 isoenzymes in the rice would result in plants with unfavourable agronomic behaviours as observed in the DawDam variety (RoyChowdury et al., 2016).

To effectively improve rice grain storage qualities, inactivation of LOX activity should be achieved without compromising nutritional content and agronomic traits. To evaluate the contribution of the LOX3 gene to lipid oxidative stability of rice bran or bran oil, gRNA-3 (GACGAGCTCCGCAACCTGCG) (SEQ ID NO:32) and gRNA-4 (CGTGCGTGCAGATCCGGACT) (SEQ ID NO:33) were designed in vector V1 in Example 5 and vector V2 in Example 6. The gRNA-3 was designed to target the 1^{st} exon, and the gRNA-4 targeted the 20 bp spanning 1^{st} intron (nt 30˜50) and 2^{nd} exon of LOX3 (nt 467˜486).

### Example 4—Identification and Isolation of FATB Genes from Rice

FATB genes encode the enzyme palmitoyl-ACP thioesterases which have the activity preferentially releasing fatty acids that have a length of 16 carbons or less from acyl-acyl carrier protein. Putative rice FATB sequences were identified using homology-based searches with Arabidopsis AtFATB sequence AtACPTE32 (NCBI access number AF213480). The program used was Megablast available with default parameters at NCBI (www.ncbi.nlm.nih.gov/). Based on the homology with AF213480 in Arabidopsis, the most similar sequences from rice identified were four OsFATB genes in the Rice Genome Annotation Project Database (http://rice.plantbiology.msu.edu/) (Ouyang et al. 2007). The four OsFATB genes were named FATB1 (LOC_Os06g05130), FATB2 (LOC_Os11g43820), FATB3 (LOC_Os02g43090) and FATB4 (LOC_Os06g39520). Each rice FATB gene comprises 6 exons.

The rice FATB sequences were translated into amino acid sequences from corresponding coding sequences (FIG. 3) and examined for the presence of the conserved motifs. Amino acid residues considered to be essential in the AtFATB sequence are generally known as the catalytic triad are the cysteine 264, asparagine 227 and histidine 229. Consistent with AtFATB1, the catalytic triad (aspartate N-227, histidine H-229 and cysteine C-264) locates at the C terminus of the FATBs shown in FIG. 2 (Yuan et al., 1996; Mayer and Shanklin, 2005). FATB1 and FATB2 contain all three catalytic amino acid residues, and FATB4 contains two of the three catalytic amino acids (N-227 and H-229) in the conserved motif NQHVNN (SEQ ID NO:38) found in FATB1 and FATB2 sequences. However, all three catalytic amino acids residues and NQHVNN (SEQ ID NO:38) are absent in FATB3.

The program CLUSTAL with default parameters was used for sequence comparison. The nucleotide sequence identity between FATB1 (LOC_Os06g05130) and FATB4 (LOC_Os06g39520) over the entire coding sequence was 64.6%, and between their deduced amino acid sequences was 54.3%. The proteins deduced from FATB1, FATB2, FATB3 and FATB4 are known to correspond to amino acid sequences of 427, 425, 298 and 357 amino acids respectively.

### Gene Editing of FATB

To evaluate the contribution of FATB1 gene to palmitic acid content in the rice grain, gRNA-5 was targeted to the beginning of the second exon of OsFATB1 (CTGAACCATGTGAAAACTGC) (SEQ ID NO:34) and included in the vector V1 (see Example 1). The function of other FATB members is uncharacterised so far. Thus, the contribution of each FATB gene to C16:0 content in the grain was further characterised by gene editing. The gene editing vector V2 contained gRNA-6-gRNA-8 (see Example 1). gRNA-6 (TCCTGGCAGCTGAGAAGCAG) (SEQ ID NO:35) and gRNA-8 (GGGCTGCTAGGAGATGGTTT) (SEQ ID NO:36) were designed for editing FATB1 and FATB4 while gRNA-7 (ATGATTCGGTCCTACGAGAT) (SEQ ID NO:37) was targeting both FATB2 and FATB3 simultaneously in a conserved region.

### Example 5—Gene Editing of Rice Fad2-1, Lox3 and FATB1 with Vector V1

Multiplex genome editing methods in rice demonstrated by Ma et al. (2016) and reviewed by Zafar et al. (2020) are being rapidly developed because of its potential to offer transgene-free methods of plant improvement by editing several genes simultaneously. However rice genetic engineering to alter the lipid profile in the bran layer of rice to enhance the utility and shelf life of wholegrain rice and to prevent rancidity of bran and RBO without the need for further processing has not been successfully achieved.

gRNA and Vector Design of V1

To construct gene editing vector V1, the inventors designed two gRNAs targeting FAD2-1 (LOC_Os02g48560), two gRNAs targeting LOX3 (LOC_Os03g49350) and one for FATB1 (LOC_Os06g05130) as described in Examples 2, 3 and 4. The vector was generated following the method described by Ma et al. (2015). Each gRNA was under the control of a rice promoter, gRNA targeting FAD2-1 driven by U3 promoter, gRNA targeting LOX3 driven by U6a promoter and gRNA targeting FATB1 driven by U6c promoter.

T-DNA binary vector V1 for CRISPR gene editing is shown in FIG. 2. The gRNA-1 targeted the 20 bp (nts 3-22) from the end of the start codon covering the second ATG, and the gRNA-2 at nts 532-551 downstream of the start codon in the FAD2-1. The gRNA-3 was designed at the nt 31 to 50 region in the first exon, and the gRNA-4 at the 20 bp (nt 467-486) crossing first intron and second exon of LOX3. The gRNA-5 was designed at the beginning of the second exon (nt 523-542) of FATB1. A schematic of the multiple gRNA expression cassette is shown and detailed plasmid map is shown in FIG. 2.

### Rice Transformation

The V1 vector was transformed into Agrobacterium strain AGL1 for rice transformation following the method described by Toki et al. (2006) with modification as per Example 1.

A total of 14 To transgenic lines carrying the hygromycin resistance gene were obtained after tissue culture of transformed calli generated from Nipponbare cv. Leaf tissue was used to prepare DNA samples from each transgenic line. DNA was subject to PCR amplification as described in Example 1 and PCR products were sequenced.

### Edited Alleles

Four edited lines were identified by sanger sequencing of PCR products amplified from the corresponding genomic DNA of the three candidate genes. The successfully edited plants are referred to herein as V1-4, V1-7, V1-12 and V1-13 (Table 3).

Line V1-4 had one mutated allele of fatb1 edited at gRNA-5 region with one nucleotide inserted leading to frame-shift which introduced a premature stop codon, while FAD2-1 and LOX3 were unchanged. The T₀ plant grew normally but the fertility was reduced.

Line V1-12 is biallelic edited carrying mutations (−7/+1 at gRNA-3, +1/+1 at gRNA-4) in the LOX3 gene, while FAD2-1 and FATB1 genes were unchanged. The lox3 mutations in line V1-12 at the target site of the gRNA-3 all led to downstream premature stop codons.

TABLE 3
V1 lines

| Gene | line | | | | | | |
|---|---|---|---|---|---|---|---|
| V1 T₀ lines | | Genotype | Allele | gRNAs | gRNAs | Amino acid sequence resulting from edit | |
| FAD2 | | | | gRNA1 (position 3-22) | gRNA2 (position 532-551) | | |
| | Nip | FAD2/LOX3/FATB1 | | Nt1′ATGGGTGCCGGCGGCAGGATG | Nt532′TACGTGTACCACAACCCGATCGG′nt554 | Aa1′MGAGGRMTE′aa9 | Aa178′YVYHNPIG′aa185 |
| | | | | ACGG′nt25 | | | |
| | V1-4 | FAD2/LOX3/fatb1 | | WT | WT | WT | WT |
| | V1-7 | fad2/lox3/FATB1 | AL1 | Nt1′ATGGGTGCCGGCGGCAGGATT | Nt533′TACGTGTACCACAACCCCGATCGG′nt556 | Aa1′MGAGGRIDC′aa9 | NA because of |
| | | | | GACGG′nt26 | | | premature stop codon |
| | | | AL2 | Nt1′ATGGGTG-------------- | Nt518′TACGTGTACCACAA---GATCGG′nt537 | Aa1′MGDGEGAGG′aa9 | NA because of |
| | | | | ACGG′nt11 | | | premature stop codon |
| | V1-12 | FAD2/lox3/FATB1 | | WT | WT | WT | WT |
| | V1-13 | fad2/lox3/FATB1 | KD | nt1′ATGGGTGCCGGCGG----ATG | nt519′TACGTGTACCACA---CGATCGG′nt547 | aa1′MGAGG*′aa5 | NA because of |
| | | | | ACGG′nt21 | | /aa1′MTE′aa3 | premature stop codon |
| | | | | | | | /Aa172′YVYHT- |
| | | | | | | | IG′aa178 |
| | | | KO | nt1′ATGGGTGCCGGCGGCAGGAGT | CGTACGTGTACCCAACCCAGAT | Aa1′MGAGGRSDG′aa9 | NA because of |
| | | | | GACGG′nt26 | | | premature stop codon |
| LOX3 | | | | gRNA3 (position 31-50) | gRNA4 (position 467-486) | | |
| | Nip | FAD2/LOX3/FATB1 | | nt31′GACGAGCTCCGCAACCTGCG | Nt467′CGTGCGTGCAGATCCGGACTCGG′nt487 | Aa11′DELRNLRGD′aa18 | Aa156′ACVQIRTR′aa163 |
| | | | | CGG′nt53 | | | |
| | V1-4 | FAD2/LOX3/fatb1 | | WT | WT | WT | |
| | V1-7 | fad2/lox3/FATB1 | AL1 | 400+ bp deletion | Unknown | unknown | unknown |
| | | | AL2 | unknown because of poor | unknown because of poor sequencing | unknown | unknown |
| | | | | sequencing | | | |
| | V1-12 | FAD2/lox3/FATB1 | AL3 | nt31′GACGAGCTCC------- | Nt460′CGTGCGTGCAGATCCGGAACTCGG′nt483 | Aa11′DELRAATT′aa18 | Aa71′PIRTNSES′aa77 |
| | | | | GCGCGG′nt46 | | | |
| | | | AL4(KO) | nt31′GACGAGCTCCGCAACCT | Nt468′CGTGCGTGCAGATCCGGAACTCGG′nt491 | Aa11′DELRNLAR′aa18 | Aa73′HRSGTRRV′aa80 |
| | | | | AGCGCGG′nt54 | | | |
| | V1-13 | fad2/1ox3/FATB1 | AL5(KO) | nt31′GACGAGCTCCGCAA-- | Nt465′CGTGCGTGCAGATCCGGAACTCGG′nt488 | Aa11′DELRNARR′aa18 | Aa72′HRSGTRRV′aa79 |
| | | | | TGCGCGG′nt51 | | | |
| | | | AL6 | nt31′GACGAGCTCCGCAACC- | Nt466′CGTGCGTGCAGATCC--ACTCGG′nt487 | Aa11′DELRNRAA′aa18 | Aa73′PIHSESRL′aa80 |
| | | | | GCGCGG′nt52 | | | |
| FATB1 | | | | gRNA5 (position 523-542) | | | |
| | Nip | FAD2/LOX3/FATB1 | | Nt623′CTGAACCATGTGAAAACTGC | NA | Aa175′LNHVKTAG′aa182 | NA |
| | | | | TCG′nt645 | | | |
| | V1-4 | FAD2/LOX3/fatbl | A11 | Nt623′CTGAACCATGTGAAAACCTG | NA | Aa175′LNHVKTCW′aa182 | NA |
| | | | | CTGG′nt646 | | | |
| | V1-7 | Isd2/1ox3/FATB1 | | WT | NA | WT | NA |
| | V1-12 | FAD2/1ox3/FATB1 | | WT | NA | WT | NA |
| | V1-13 | fad2/lox3/FATB1 | | WT | NA | WT | NA |

Lines V1-7 and V1-13 are biallelic heterozygous at the FAD2-1 and LOX3 loci meaning both the FAD2-1, and LOX3 were edited, but the FATB1 sequence was confirmed as wild type. The mutations in V1-7fad2 resulted in an insertion in Allele 1 and a 14-bp deletion in Allele 2 in the gRNA-1 region (+1/−14 at gRNA-1), as well as nucleotide substitution in Allele 1 and a 43-bp deletion in Allele 2 in the gRNA-2 target region (A→C/−43 at gRNA-2). The gene edits resulted in premature stop codon in the downstream of gRNA-1 region in FAD2-1. V1-7 T₀ plants grew normally but were considered sterile. Previously Zaplin et al. (2013) showed that RNAi suppression could successfully suppress FAD2-1 expression in a tissue-specific manner that resulted in higher oleic rice compare to wildtype. Recently, no significant agronomic defect was reported in the high oleic rice FAD2-1 knock out (KO) mutants generated by Abe et al. (2018) using CRISPR-CAS9 gene editing technology, however no supporting plant growth data was presented. In contrast, knocking out Camelina sativa FAD2-1, FAD2-2 and FAD2-3 showed significant plant growth defects (Morineau et al., 2017). According to our observation, the complete knockout (KO) mutations in rice FAD2-1 gene induced T₀ plant infertility most likely due to the lack in C18:3n3 in anther as discussed below.

FAD2-1 editing in the V1-13 line resulted in a small deletion in Allele 1 and single nucleotide insertion in Allele 2 in the gRNA1 region (−4/+1 at gRNA-1) as well as in the gRNA2 region (−3/+1 at gRNA-2) resulting in the deletion of between 1 to 10 amino acids in the downstream of gRNA-1 region of fad2-1 this was designated as the fad2 knock down (KD) or fad2-KD. The resulting fad2-KD encodes a truncated protein with a deletion of the first six amino acid residues from the N terminus (from gRNA1 editing). In addition, a mutation of N182/P183 to T182 was generated by the gRNA2 editing.

V1-12 and V1-13 were allowed to self pollinate with the aim of segregating the edited alleles of fad2-1 and lox3 in the T₁ progeny. Plants that were homozygous for lox3, for Allele 1 and Allele 2 (Table 3) in single gene KO mutants were obtained from the T₁ and T₂ progenies of V1-12. Allele or ‘Al’ refers to the edited alleles at the same locus. Plants that were homozygous for fad2-1 KD were identified in T₁ and T₂ progeny of V1-13 in combination with lox3-Allele 5 or Allele 6. Whereas, fad2-1 Allele 2 was only found with fad2-1 Allele 1 in the heterozygote progeny of V1-13 by Sanger sequencing.

TABLE 4
V2 T₂ lines.

| Gene | Line | Genotype | Allele | gRNA target sequence | Amino acid sequence |
|---|---|---|---|---|---|
| FATB1 | | | | gRNA6 (position 323-342) | |
| | Nip | | | TTTCCTGGCAGCTGAGAAGCAGTGG | FLAAEKOW |
| | V2-2-2.3-4 | fatb4 | | WT | WT |
| | V2-12-1.2-13 | fatb1/2 HE | | TTTCCTGGCAGCTGAGAA -CAGTGG | FLAAENSG |
| | V2-12-1.2-12 | fatb1/4 | | TTTCCTGGCAGCTGAGAA -CAGTGG | FLAAENSG |
| | V2-8-4.1-3 | fatb2/3 | | WT | WT |
| | V2-12-1.1-4 | fatb1/3/4 | A12 | TTTCCTGGCAGCTGAGAA -CAGTGG | FLAAENSG |
| | V2-26-1.1-3 | fatb2/3/4 | | WT | WT |
| | V2-28-4.4-11 | fatb 1 HE/ | | TTTCCTGGCAGCTGAGAAGACAGTGG / WT | FLAAEKTV |
| | | 2/3/4 | | | |
| FATB2 | | | | gRNA7 (position 451-470) | |
| | Nip | | | TTCATGATTCGGTCCTACGAGATTGG | MIRSYEIGAD |
| | V2-2-2.3-4 | fatb 4 | | WT | WT |
| | V2-12-1.2-13 | fatb 1/2HE | | TTCATGATTCGGTCCTACGAAGATTGG / WT | MIRSYEDWC* / WT |
| | V2-12-1.2-12 | fatb 1/4 | | WT | WT |
| | V2-8-4.1-3 | fatb 2/3 | A11 | TTCATGATTCGGTCCTACGAAGATTGG | MIRSYEDWC* |
| | V2-12-1.1-4 | fatb 1/3/4 | | WT | WT |
| | V2-26-1.1-3 | fatb 2/3/4 | A12 | TTCATGATTCGGTCCTACGAAGATTGG | MIRSYEDWC* |
| | V2-28-4.4-11 | fatb | | 156 bp deletion | --------- |
| | | 1HE/2/3/4 | | | 26/08/2020 |
| FATB3 | | | | gRNA7 (position 451-470) | |
| | Nip | | | TTCATGATTCGGTCCTACGAGATTGG | MIRSYEIGAD |
| | V2-2-2.3-4 | fatb 4 | | WT | WT |
| | V2-12-1.2-13 | fatb1/2HE | | WT | WT |
| | V2-12-1.2-12 | fatb 1/4 | | WT | WT |
| | V2-8-4.1-3 | fatb 2/3 | A11 | TTCATGATTCGGTCCTACGAAGATTGG | MIRSYEDWC* |
| | V2-12-1.1-4 | fatb1/3/4 | A11 | TTCATGATTCGGTCCTACGAAGATTGG | MIRSYEDWC* |
| | V2-26-1.1-3 | fatb2/3/4 | A11 | TTCATGATTCGGTCCTACGAAGATTGG | MIRSYEDWC* |
| | V2-28-4.4-11 | fatb | | TTCATGATTCGGTCCTACGA - - TTGG | MIRSY-DWR* |
| | | 1HE/2/3/4 | | | |
| FATB4 | | | | gRNA8 (position 624-643) | |
| | Nip | | | GCTGGGCTGCTAGGAGATGGTTTTGG | GLLGDGFG |
| | V2-2-2.3-4 | fatb 4 | A11 | GCTGGGCTGCTAGGAGAT - - TTTTGG | GLLGDFWL |
| | V2-12-1.2-13 | fatb1/2HE | | WT | WT |
| | V2-12-1.2-12 | fatb 1/4 | | GCTGGGCTGCTAGGAGATGGTTTTTGG | GLLGDGFW |
| | V2-8-4.1-3 | fatb 2/3 | A12 | GCTGGGCTGCTAGGAG AT- - -TTTGGC | GLLGD-FG |
| | V2-12-1.1-4 | fatb 1/3/4 | A11 | GCTGGGCTGCTAGGAGAT - - TTTTGG | GLLGDFWL |
| | V2-26-1.1-3 | fatb 2/3/4 | A11 | GCTGGGCTGCTAGGAGAT - - TTTTGG | GLLGDFWL |
| | V2-28-4.4-11 | fatb | | GCTGGGCTGCTA - - - - - - - - TTTTGG | GLLFWLNA |
| | | 1HE/2/3/4 | | | |

TABLE 5
Summary of resulting genotypes and sterility.

| Line | Genotype | Generation | Panicle sterility |
|---|---|---|---|
| V1-7 | FAD2-KO/KO | T₀ | 49/49, 100% |
| V1-13-1.5 | FAD2-KD/KO | T₁ | 10/43, 13.5% |
| V1-13-1.4 | FAD2-KD/KO | T₁ | 2/43, 4.6% |
| V1-13-1.5-8 | FAD2-KD/KO | T₂ | 9/57, 15.8% |
| V1-13-1.5-11 | FAD2-KD/KO | T₂ | 7/59, 11.9% |
| V1-13-1.5-10 | FAD2-KD | T₂ | 7/52, 13.5% |
| V1-13-6.5-6 | FAD2-KD | T₂ | 6/88, 6.8% |
| V1-13-6.5-4 | FAD2-KD | T₂ | 9/89, 10.1% |
| V1-12-1.6-4 | LOX3-KO | T₂ | 5/56, 8.9% |
| V1-12-1.6-7 | LOX3-KO | T₂ | 8/98, 8.1% |
| V2-8-2.2-1 | Neg control | T₂ | 2/63, 3.2% |
| V2-16-2.3-1 | Neg control | T₂ | 4/69, 5.8% |

### Growth Phenotype

Fertility of the V1-13 selfed lines was assessed by panicle sterility. To consider the effect of lox3-KO the V1-12 selfed lines were also included. The panicle with most florets developed was selected and labelled for each plant. After seeds matured on plant, the panicle was collected and dried at 37° C. The number of sterile florets was recorded and the sterility was calculated as a percentage by dividing the total floret number. Plants were grown in the glasshouse with a day/night temperature of 26° C./22° C. The panicle sterility was higher in the lines with a fad2-1KO (Table 5). No significant differences regarding the seed physical characteristics and appearance were observed.

### Example 6—Multiple FATB Gene Editing with Vector V2 gRNA and Vector Design of V2

Previous results indicated that genes encoding the FATB (Fatty acyl-ACP Thioesterases B) isoforms differed in their function and prevalence in plant tissues (Zaplin et al., 2013). To evaluate the contribution of each FATB gene to seed oil content and composition the V2 vector was designed. The V2 vector was designed to create a mutation in the corresponding target genes: FATB1 (LOC_Os06g05130), FATB2 (LOC_Os11g43820), FATB3 (LOC_Os02g43090) and FATB4 (LOC_Os06g39520). The V2 vector comprised the gRNA6, gRNA7 and gRNA8; with gRNA7 targeting both FATB2 and FATB3 simultaneously in a conserved common regions as described in Examples 1 and 4. The experiment was undertaken to attempt to knockout the respective FATB enzyme activity in the resulting plant.

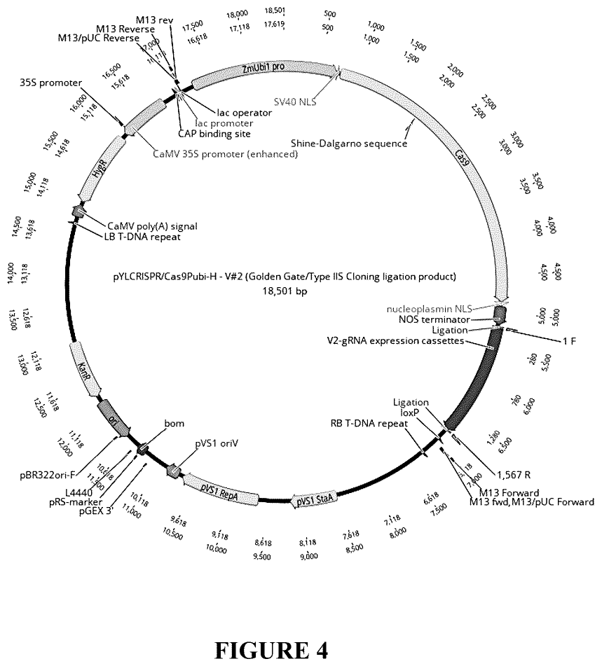

The method as described in Ma et al. (2015) was followed to generate the vector and modified as described in Example 1. Each gRNA was placed under control of a rice promoter; the U3 promoter was used to drive gRNA6 expression, the U6a promoter was used to drive gRNA7 expression and the U6b promoter was used for gRNA8. A schematic of the T-DNA binary vector and plasmid map for transformation is shown in FIG. 4.

### Rice Transformation

The V2 vector was transformed into Agrobacterium strain AGL1 for rice transformation following the method described by Toki et al. (2006) with modifications as per Example 1.

Gene edited T₀ V2 lines were identified in the V2 mutant population by Sanger sequencing as described in Example 1 and 5. Multiple fertile plants were observed and four lines V2-2, V2-8, V2-12 and V2-26 were selected for analysis. The T₀ V2 lines were grown in a glasshouse and allowed to self-pollinate until T₃ generation (Table 4). Progeny of four lines were identified as carrying 6 different combinations of the 4 OsFATB KO alleles as shown in (Table 6). All of the FATB isoforms were successfully edited.

TABLE 6
Combinations of fatb knockout (KO) mutants
carried by the selected T₂ V2 lines.

| Line | Genotype | FATB-1 | FATB-2 | FATB-3 | FATB-4 |
|---|---|---|---|---|---|
| V2-2-2.3-4 | fatb 4 | WT | WT | WT | KO |
| V2-8-4.1-3 | fatb 2/3/4 | WT | KO | KO | WT |
| V2-12-1.1-4 | fatb1/3/4 | KO | WT | KO | KO |
| V2-26-1.1-3 | fatb 2/3/4 | WT | KO | KO | KO |
| V2-12-1.2-13 | fatb 1/2HE | KO | HE | WT | WT |
| V2-12-1.2-12 | fatb 1/4 | KO | WT | WT | KO |
| V2-8-2.2 | Neg | WT | WT | WT | WT |

Note:
the genotype column indicates the FATB gene modified by the gene editing. WT, HE and Neg are wild type, heterozygous and negative control respectively.

### DISCUSSION

Generally, it was observed that the mutations were 1 or 2 base pair insertion or deletion which resulted in a frameshift mutation. Where the editing caused a 3 base pair insertion or deletion this resulted in one amino acid addition or deletion (see for example line V2-8-4.1-3) in the protein. It was expected that an amino acid deletion or insertion is not likely to impact enzyme activity unless it occurs in an essential region that affects the 3D structure or enzyme activity. Incidence of sterility seemed to be associated with the fatb1/2 genotype due to the observation that none of the progeny was identified as fatb1/2 homozygote.

### Example 7—Fatty Acid Profile of Gene Edited Rice Plants

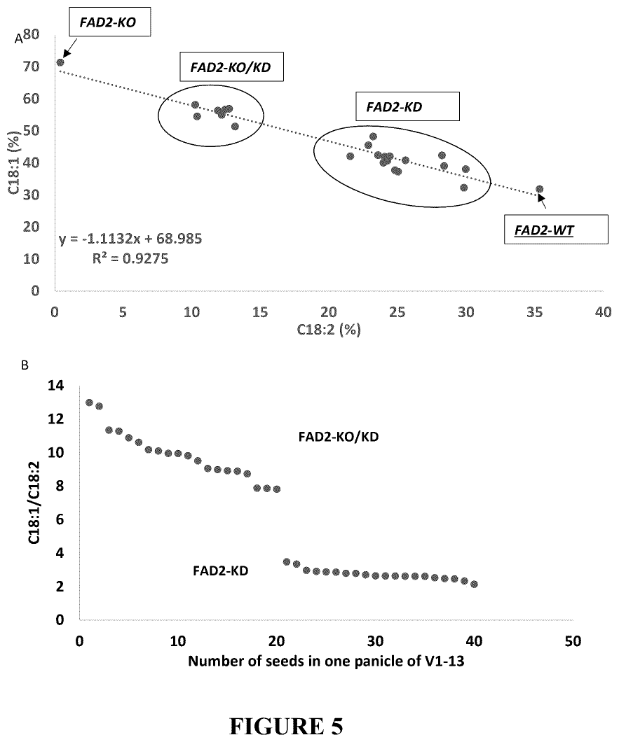

To analyse the effect of the gene editing on fatty acid composition, total lipid was isolated from grain, anther, leaf and root samples of the V1 transformed rice plants and a negative segregated control line V2-8-4.1-3 (‘Neg’). Fatty acid composition was determined for each lipid extract by GC-FID as described in Example 1. The results are presented in Table 6 and some of that data is presented graphically in FIG. 5. The proportion of each fatty acid was expressed as a percentage of the total fatty acid in the seed oil of the grain as determined by GC as described in Example 1.

TABLE 6
Total fatty acid composition in the seed, anther, leaf and root tissues of V1 mutants.

| | Rice plant | C14:0 | C16:0 | C16:1 | C18:0 | C18:1 | C18:1d11 | C18:2 | C18:3n3 |
|---|---|---|---|---|---|---|---|---|---|
| Sample | LINE | (%) | (%) | (%) | (%) | (%) | (%) | (%) | (%) |
| Seed | | | | | | | | | |
| fad2-KO + lox3-KO | V1-13-3.4 | 0.16 | 10.73 | 0.30 | 3.21 | 82.19 | 0.00 | 1.55 | 0.35 |
| fad2-KO/KD + lox3-KO | V1-13-1.4 | 0.50 | 16.42 | 0.18 | 2.48 | 68.13 | 1.02 | 9.99 | 0.73 |
| fad2-KD + lox3-KO | V1-13-1.4 | 0.91 | 21.14 | 0.19 | 3.23 | 55.31 | 1.02 | 15.93 | 1.07 |
| FAD2-WT + lox3-KO | V1-12-1.6 | 1.12 | 23.01 | 0.00 | 2.99 | 26.36 | 1.01 | 43.95 | 1.57 |
| FAD2-RNAi | Line D | 0.34 | 20.03 | 0.24 | 2.60 | 53.26 | 1.42 | 18.89 | 1.11 |
| Neg | V2-8-2.2 | 0.77 | 22.66 | 0.15 | 2.71 | 28.51 | 2.21 | 40.18 | 1.20 |
| Anther | | | | | | | | | |
| fad2-KO + lox3-KO | V1-7 | 0.00 | 15.48 | 0.00 | 5.11 | 61.92 | 0.00 | 0.00 | 9.98 |
| fad2-KO/KD + lox3-KO | V1-13-1.4 | 0.34 | 21.22 | 0.00 | 9.65 | 19.49 | 0.78 | 9.03 | 37.25 |
| fad2-KD + lox3-KO | V1-13-1.4 | 0.40 | 25.01 | 0.00 | 10.36 | 4.68 | 0.59 | 9.23 | 48.14 |
| FAD2-WT + lox3-KO | V1-12-1.6 | 0.42 | 26.22 | 0.00 | 12.17 | 1.23 | 0.54 | 8.58 | 49.08 |
| Neg | V2-8-2.2 | 0.48 | 26.24 | 0.00 | 10.68 | 1.14 | 0.49 | 8.49 | 50.19 |
| Leaf | | | | | | | | | |
| fad2-KO + lox3-KO | V1-7 | 0.00 | 10.33 | 2.16 | 0.00 | 43.03 | 0.00 | 2.50 | 37.29 |
| fad2-KO/KD + lox3-KO | V1-13-1.4 | 0.00 | 15.19 | 3.07 | 0.00 | 7.97 | 0.00 | 7.96 | 62.53 |
| fad2-KD + lox3-KO | V1-13-1.4 | 0.00 | 13.40 | 3.10 | 1.74 | 2.82 | 0.00 | 11.15 | 64.76 |
| FAD2-WT + lox3-KO | V1-12-1.6 | 0.43 | 14.46 | 1.85 | 2.06 | 1.34 | 0.16 | 11.25 | 65.30 |
| Neg | V2-8-2.2 | 0.84 | 17.05 | 2.68 | 0.66 | 1.66 | 0.59 | 11.90 | 61.50 |
| Root | | | | | | | | | |
| fad2-KO + lox3-KO | V1-7 | NA | NA | NA | NA | NA | NA | NA | NA |
| fad2-KO/KD + lox3-KO | V1-13-1.4 | 1.26 | 17.63 | 0.29 | 2.50 | 31.36 | 0.72 | 22.05 | 9.30 |
| fad2-KD + lox3-KO | V1-13-1.4 | 0.43 | 24.05 | 0.10 | 1.22 | 14.64 | 0.54 | 34.26 | 11.61 |
| FAD2-WT + lox3-KO | V1-12-1.6- | 0.40 | 23.77 | 0.27 | 3.33 | 6.94 | 1.55 | 35.03 | 14.70 |
| Neg | V2-8-2.2 | 0.55 | 24.75 | 0.23 | 1.96 | 5.95 | 0.46 | 43.13 | 11.15 |

It was generally observed that, the mutants carrying edited fad2-1 alleles exhibited altered fatty acid composition. However, the fatty acid composition was relatively unchanged in mutants with only lox3 was edited when compared to that of negative control (Neg). In the mature seeds, it was observed that the increase in C18:1 was at the expense of C18:2 and C16:0 in all the fad2-1 mutants compared with Neg. The most significant increase in C18:1 was found in the fad2-1 KO lox3 KO line (54% increase) and smallest increase in the fad2-1 KD lox3 KO line (27% increase) with fad2-1 KO/KD (40% increase) observed to be between the two oil profiles. Notably, the C18:2 decreased to under 1% in fad2-1KO lox3-KO and a 4-fold reduction in the fad2-1 KD/KO line was observed. Surprisingly, the proportion of the C18:3n3 in the fad2 mutated lines was reduced to below 1% of total FFA in the fad2-1KO and fad2-1 KO/KD lines but was relatively unchanged in the fad2-1 KD homozygous line when compared to the control.

Anthers of the control Nipponbare cv. were found to contain approximately 50% C18:3n3, 26% C16:0, 9% C18:2 and 11% C18:0. In comparison to seed tissue, the anthers and leaf oil content from plants containing higher C18:1 had a major trade-off in C18:3n3 and C18:2 in the fad2-1 mutants. For example the fatty acid composition from the anthers of fad2-1 KO lines was approximately 62% C18:1 and exhibited a dramatic reduction in C18:2 and C18:3n3. In the fad2-1 KO/KD, the variation in C18:1 was less pronounced but with still 18% increase in the anther and 6% increase in the leaf at the expense of C18:3n3 and C18:2, respectively. While fad2-1 KD showed only marginal difference in the fatty acid composition compared to the control.

In the root tissue, fad2-1 KO/KD line oil contained remarkably higher level of C18:1, 5-fold higher than the control, and a reduction in both C16:0 and C18:2 (7% and 21%). The changes in oil composition derived from the fad2-1 KD was less pronounced with a smaller increase in C18:1 at the expense of C18:2. In all tissue types, the fatty acid composition of FAD2 WT/lox3KO was found to be comparable to the negative control.

### Total Fatty Acid Composition of T3 Single Seeds of the V1-13

From the V1-13 line that was self-crossed for further generations, half seed FAC was conducted to investigate the inheritance of the phenotype changes in C18:1 and C18:2 content and fatty acid ratio in mutant V1-13. All V1-13 lines and progeny contained a mutated LOX3 gene (KO) resulting in no expression of the LOX3 protein. Seed-to-seed variation of C18:1 and C18:2 ratio was observed in the seeds of fad2-1KO/KD.

It appeared the C18:1 and C18:2 contents of individual seeds were highly correlated regardless of fad2-1 genotypes (FIG. 5). The C18:2 level was reduced from 35% to nearly null, along with C18:1 content increased from about 30% to 70%. This observation was reported in FAD2 RNAi transgenic plants of cotton, Arabidopsis and rice previously (Chapman et al., 2001; Zaplin et al., 2013; Stoutjesdijk et al., 2002) and regarded as a variability in the extent of suppression. Without manipulation of the FAD2-1 gene expression in the CRISPR mutant, our results suggest the FAD2 enzyme activity may be unstable to some extent in the heterozygote during seed development for some reason. Seeds containing over 70% C18:1 were scarcely spotted in hundreds of T₁ to T₃ seeds, which further indicated fad2-1 KO homozygotes may be inferior in fertility and less useful agronomically. Out of 43 florets in one panicle of V1-13 T₁ plant, 3 florets failed in seed development and 40 developed into viable seeds, in which the ratio (C18:1/C18:2) was between 8 to 13 in 20 sampled seeds and between 2 to 4 in the other 20 seeds (FIG. 5). The corresponding genotype of fad2-1 in the lines were confirmed as fad2-1 KO/KD and fad2-1 KD/KD, respectively.

### Germination Rate

To analyse the effect of the mutations of the FAD2 gene on the seed fertility recovered seeds were grown in pots as per Example 1.

After the plants matured and dried, a panicle from each of the fad2-1KD+lox3-KO, FAD2-WT+lox3-KO and wildtype Nipponbare (Neg) was soaked in water in the glasshouse for two weeks. Seeds on the panicle of fad2-KD+lox3-KO exhibited a higher germination rate and vigour compared with FAD2-WT+lox3-KO and NEG. The germination rate was observed as follows.

| Line genotype | Viable seeds/total seeds | Germination rate |
|---|---|---|
| FAD2-WT + lox3-KO | 57/68 | 83.8% |
| fad2-KD + lox3-KO | 78/85 | 91.8% |
| Neg (Control) | 57/85 | 67.1% |

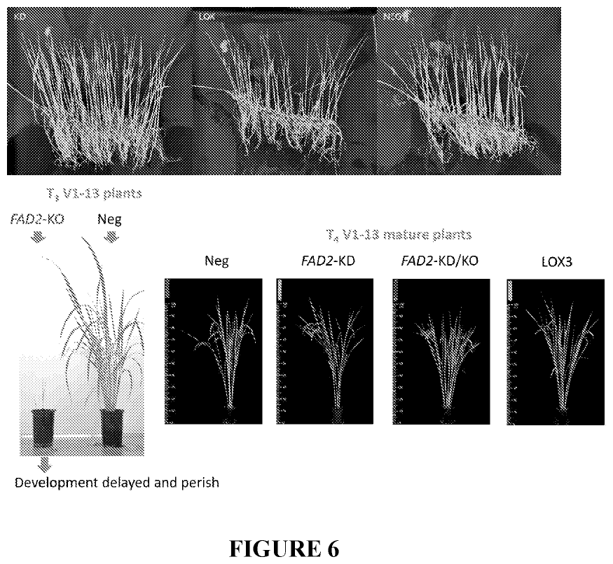

To observe the growth habit, seeds were grown in pots as described in Example 1. The mutant lines carrying the fad2-1 KO/KO alleles exhibited retard in seed germination and plant development (FIG. 6). The homozygous fad2-1 KO/KO mutants were not able to survive in the normal growing conditions. In comparison the heterozygous and homozygous KD mutants were able to mature and set seeds without obvious agronomic difference. The plant height of the fad2-1 KD/KO was slightly shorter comparing with other mutants and negative control.

### DISCUSSION

The membrane lipids of the cells in various tissues require unsaturated fatty acids (UFAs) to meet the fatty acid balance for maintenance of cell functions allowing the plant to cope with environmental stress (He and Ding, 2020). Lipids and their derivatives (fatty acids, waxes and phospholipids) are crucial for pollen wall maturation and viability (Shi et al., 2015). A recent study reported down-regulation of GhFAD2-3 can result in male sterility of cotton because of remarkably reduced linoleic acids in the anther wax and cutin (Liu et al., 2019). The anther development was impaired with shrunken abnormal outer surface (Liu et al., 2019). The fatty acid composition of anthers of V13 progeny showed a remarkable increase in oleic acid along with a reduction in palmitic and linolenic acids at different levels in the corresponding fad2 mutant genotypes (Table 7).

The sterility rate in a single panicle varied in the progenies of the mutant lines, some of which were more than double the rate of the negative controls. The seed set also varied with some lines producing more seeds and others producing less seeds than the negative controls.

In this study the inventors observed fad2 allelic segregation pattern correlated with sterility. The fad2 KO and KD alleles segregated in the V1-13 progeny influencing the oleic acid content of the fatty acids (Table 7). It was found that among the seeds of V1-13 progeny the fad2-KO/KD lines contained about over 68% oleic acid and the rest of the progeny which resulted in a fad2-KD genotype contained about 55% oleic acid. One recovered line with the fad2-KO homozygous genotype contained over 82% oleic acid (Table 7). This suggests knocking out FAD2 gene leads to high sterility in rice.

### Example 8—Total Fatty Acid Composition of T3 Single Seeds of the V2 Mutants

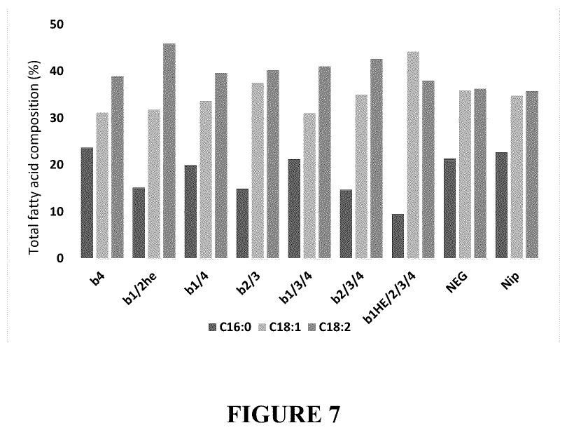

Previously analysis of genes encoding the FATB isoforms in rice showed that FATB1 (LOC_Os06g05130) and FATB2 (LOC_Os11g43820) were more highly expressed in the grain than the other two genes. To analyse the effect of the gene editing on fatty acid composition, the healthy recovered V2 lines were propagated to T₃ population. Total lipid was isolated from grain of the V2 transformed rice plants and a negative control line V2-8-2.2. Fatty acid composition was determined for each lipid extract by GC-FID as described in Example 1. The data are presented in Table 8 and some of that data is presented graphically in FIG. 7. The relative proportion of each fatty acid was expressed as a percentage of the total fatty acid in the seed oil of the grain as determined by GC as described in Example 1.

TABLE 8
Total fatty acid composition in the seed and leaf tissues of V2 mutant lines.

| | | | | | | | | | |
|---|---|---|---|---|---|---|---|---|---|
| Genotype | sample | C14:0 | C16:0 | C16:1 | C18:0 | C18:1 | C18:1d11 | C18:2 | C18:3n3 |

seed

| | | | | | | | | | |
|---|---|---|---|---|---|---|---|---|---|
| fatb4 | V2-2-2.3-4 | 0.00 | 23.68 | 0.21 | 2.62 | 31.18 | 0.97 | 38.91 | 1.32 |
| fatb1/2he | V2-12-1.2-13 | 0.00 | 15.14 | 0.30 | 3.02 | 31.84 | 1.18 | 45.91 | 1.33 |
| fatb1/4 | V2-12-1.2-12 | 0.00 | 19.91 | 0.23 | 2.79 | 33.72 | 0.99 | 39.56 | 1.36 |
| fatb2/3 | V2-8-4.1-3 | 0.00 | 14.94 | 0.27 | 3.18 | 37.57 | 1.10 | 40.23 | 1.34 |
| fatb1/3/4 | V2-12-1.1-4 | 0.00 | 21.23 | 0.25 | 2.85 | 31.08 | 1.02 | 41.02 | 1.33 |
| fatb2/3/4 | V2-26-1.1-3 | 0.00 | 14.71 | 0.30 | 3.59 | 35.06 | 1.14 | 42.65 | 1.34 |
| fatb1HE/2/3/4 | V2-8-2.2-1 | 0.00 | 9.48 | 0.00 | 4.64 | 44.21 | 1.01 | 37.99 | 1.80 |
| NEG | V2-2-2.3-4 | 0.00 | 21.36 | 0.23 | 2.43 | 35.94 | 1.04 | 36.23 | 1.24 |

Leaf

| | | | | | | | | | |
|---|---|---|---|---|---|---|---|---|---|
| fatb4 | V2-2-2.3-4 | 0.00 | 16.61 | 2.62 | 1.45 | 0.00 | 0.00 | 14.18 | 62.90 |
| fatb1/2HE | V2-12-1.2-13 | 0.00 | 13.60 | 3.03 | 1.24 | 0.00 | 0.00 | 16.30 | 63.50 |
| fatb1/4 | V2-12-1.2-12 | 0.00 | 15.53 | 3.25 | 0.49 | 0.00 | 0.00 | 11.39 | 67.06 |
| fatb2/3 | V2-8-4.1-3 | 0.84 | 14.42 | 2.60 | 1.13 | 1.68 | 0.00 | 15.74 | 61.31 |
| fatb1/3/4 | V2-12-1.1-4 | 0.00 | 15.77 | 2.89 | 1.40 | 0.00 | 0.00 | 14.69 | 63.30 |
| fatb2/3/4 | V2-26-1.1-3 | 0.97 | 14.61 | 2.79 | 1.29 | 1.11 | 0.00 | 12.43 | 64.36 |
| fatb1HE/2/3/4 | V2-28-4.4-11 | 0.00 | 10.83 | 1.55 | 0.00 | 2.36 | 0.00 | 22.65 | 60.41 |
| NC | V2-8-2.2-1 | 0.82 | 17.14 | 2.72 | 1.67 | 0.59 | 0.00 | 11.84 | 61.34 |

*For ease of reference the 1, 2, 3 and 4 refers to the FATB1, FATB2, FATB3 and FATB4 genes respectively.

Comparing the C16:0 (palmitic) content of the 6 selected V2 mutants, it is striking the significant 60% reduction was shown in the fatb2/3 line and 55% reduction in the fatb 2/3/4 line compared to the negative control. Similarly, in comparison to the negative control a significant reduction (about 32%) in palmitic acid content was also observed in the fatb1/2 HE mutant line which contained ˜17.5% palmitic FAC compared to about 26.6% in the negative control (FIG. 7). The variation in C16:0% content between fatb4, fatb1 and fatb1/4 HE was not significant when compared with the negative control (Neg). This result suggests that the fatb4 knockout was not directly contributing to the palmitic content of the seed. The fatb1 knockout resulted in non-significant reductions of the palmitic content in the seed when in combination with the fatb4 mutations. The function of each FA TB gene member was not clear in rice prior to this work. This data demonstrates that the FATB2 gene is the major gene in the FATB family contributing to the C16:0 content in the seed.

### Example 9—Breeding with the Mutant Rice Lines

The V1 and V2 lines can be used to develop genetically varied genetic background or germplasm with no detrimental agronomical effects and with the benefit of rice grain containing improved nutritional composition and improved shelf-life. Two independent experiments were undertaken; (i) to develop a genetic background in Indica rice and (ii) to combine the V1 and V2 mutants.

### Indica

To test if the low C18:2 effected sterility in the Indica variety the mutant V1-13 which contained both fad2-1 KO/KD and lox3 KO mutations was crossed with Indica rice variety IR36ae.

Some fertile lines were observed in the progeny and seed was collected. Two F1 seeds were grown and plants were allowed to mature, it was noted that only a few seeds were collected from these F1 lines. Total fatty acid composition was tested as described in Example 1. Lines ae1.5-1 and ae1.5-2 were found to comprise C18:1 content of 74.6% and 72.8% and dramatically reduced C18:2 content of 1.6% and 1.8% respectively. The palmitic content was reduced from 21.1% in the negative control compared to 16.5% and 17.4% respectively in the experimental lines ae1.5-1 and ae1.5-2. Further work is underway to examine if Indica rice is a desirable background for the super high oleic acid mutated rice.

### V1-13 and V2-12 Crosses

A cross was performed between V1-13 and V2-12 TO plants to combine the edited alleles of OsFAD2-1, OsLOX3 and OsFatB in the progeny. V1-13 contained both a OsFad2-KO/KD and OsLox3-KO mutation genotype, and V2-12 which comprises a mutated FatB2/3/4 genotype. A panicle of V1-13 was randomly chosen for cross one day before anthesis. The florets were cut open by removing top ⅓ of the petals using scissors. The anthers of each floret were removed using forceps without damaging the stigma. The panicle was then contained in an envelope on the plant to avoid contamination. On the next day, the 2-5 anthers of V2-12 were collected with forceps when the anthers extended out of petals to shad pollens. The anthers were then put into the V1-13 florets with gentle shaking. The practice was repeated for three consecutive days to all the florets on the panicle.

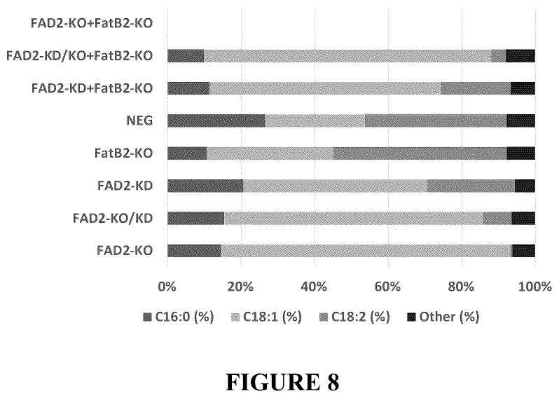

The F1 plants, named LFF, were confirmed by sanger sequencing as carrying OsFAD2-1-KD, OsLOX3-KO, OsFatB1-KO, OsFatB2-KO, OsFatB4-KO from V2-12 T₀ plant. A new type of OsFAD2-1-KO (Table 9) was identified in the progeny of the cross. The new OsFAD2-1-KO contained 22 bp deletion at gRNA1 and 17 bp deletion at gRNA2. The presence of the new allele indicated the V1 CRISPR-Cas9 remained present and active, targeting the OsFAD2-1 locus to create a new edit. The half seed fatty acid composition analysis of F2 seeds from LFF presented different profiles of seed lipids from V1 and V2 populations (FIG. 8). The OsFAD2-KD/KO+OsFatB2-KO and OsFAD2-KD+OsFatB2-KO showed about further 5-10% reduction in C16:0, 8-13% increase in C18:1, and 4-5% decrease in C18:2 from OsFAD2-KD/KO and OsFAD2-KD. Surprisingly the combination with the FAD2 KD/KO and FATB2-KO achieved a similar oleic acid (C18:1) level as the V1 FAD2-KO line, but improved from a further reduction in palmitic acid (C16:0) content from the combination of the FATB2-KO and reduction in C18:2 to around 4% total FFA.

F2 population was genotyped using PCR and sanger sequencing as described in Example 1. Progenies with OsFAD2-1-KD but new OsFAD2-1-KO segregated out, with either OsLOX3-KO, OsFatB1-KO, OsFatB2-KO, OsFatB4-KO were selected, designated as LEF-KD1, LEF-KD3 and LEF-KD5 for further analysis in F3 seeds. Genotype for KD1 and KD3 was OsFAD2-1-KD, OsLOX3-KO, FatB1-KO, FatB4-KO, KD5 was OsFAD2-1-KD, OsLOX3-KO, FatB2-KO, FatB3-KO, FatB4-KO. F3 plants were again grown to analyse F4 seeds. Fatty acid profile analysis for both F4 pooled seeds and single seed was performed. The results (Table 10) suggested that the LEF-KD1, LEF-KD3 and LEF-KD5 might be homozygous in F4 seeds.

TABLE 9
Genotype and amino acid sequence at OsFAD2-1 gRNA regions of F1 plant
of LFF (a cross between V1-13 and V2-12)

| Allele | gRNA1 | gRNA2 | Amino acid sequence resulting from edit | |
|---|---|---|---|---|
| | Nt1′ATGGGTGCCGGC | Nt529′CCGTACGTGTACCA | Aa1′MGAGGRMT | Aa177′PYVYHNPIG′aa185 |
| | GGCAGGATGACGGAGA | CAACCCGATCGGC′nt555 | EKEREE aa14 | |
| | AGGAGCGGGAGGAG′n | | | |
| | t42 | | | |
| AL1-KO | Nt1′ATGGGTGCCGGC | Nt529′CCGTA--------- | Aa1′MGAGGRSS | Aa177′PYRP′aa180 |
| | GG-------------- | --------TCGGC′nt538 | R′aa9 | |
| | --------GAGGAG′ | | | |
| | nt20 | | | |
| AL2-KD | Nt1′ATGGGTGCCGGC | Nt529′CCGTACGTGTACCA | MGAGG*′aa5 | Aa177′PYVYH-TIG′aa184 |
| | GG--- | CA---CGATCGGC′nt552 | /aa1′MTE′aa3 | |
| | ATGACGGGAGAAGGAG | | | |
| | CGGGAGGAG′nt39 | | | |

TABLE 10
Seed fatty acid profile of selected LEF progenys.

| | | | | | | | | |
|---|---|---|---|---|---|---|---|---|
| Sample | C14:0 | C16:0 | C16:1 | C18:0 | C18:1 | C18:1d11 | C18:2 | C18:3n3 |

Pooled seeds

| | | | | | | | | |
|---|---|---|---|---|---|---|---|---|
| TA2 | 0.5 | 21.7 | 0.2 | 3.3 | 45.2 | 2.5 | 25.4 | 1.2 |
| ZH11 | 1.1 | 24.6 | 0.3 | 2.9 | 32.8 | 1.8 | 35.5 | 1.1 |
| F3 seeds LEF-KD1 | 0.1 | 11.5 | 0.4 | 3.1 | 56.7 | 2.4 | 24.2 | 1.4 |
| F3 seeds LEF-KD3 | 0.5 | 16.1 | 0.3 | 3.0 | 57.0 | 2.7 | 19.1 | 1.2 |
| F3 seeds LEF-KD5 | 0.2 | 10.8 | 0.3 | 3.0 | 63.5 | 2.5 | 18.1 | 1.6 |
| F4 seeds LEF-KD1-1 | 0.5 | 17.8 | 0.3 | 3.1 | 50.7 | 3.2 | 23.2 | 1.2 |
| F4 seeds LEF-KD1-2 | 0.2 | 10.5 | 0.4 | 3.0 | 57.9 | 2.7 | 23.8 | 1.5 |
| F4 seeds LEF-KD1-3 | 0.2 | 10.9 | 0.4 | 3.0 | 58.6 | 2.6 | 22.7 | 1.6 |
| F4 seeds LEF-KD1-4 | 0.5 | 16.4 | 0.3 | 3.1 | 53.2 | 2.0 | 23.1 | 1.3 |
| F4 seeds LEF-KD1-5 | 0.3 | 13.5 | 0.3 | 2.9 | 54.9 | 2.2 | 24.6 | 1.3 |
| F4 seeds LEF-KD1-6 | 0.2 | 10.7 | 0.4 | 2.9 | 60.9 | 2.3 | 20.8 | 1.7 |
| F4 seeds LEF-KD3-1 | 0.5 | 16.0 | 0.3 | 3.1 | 55.4 | 1.9 | 21.3 | 1.5 |
| F4 seeds LEF-KD3-2 | 0.6 | 19.6 | 0.3 | 3.3 | 49.4 | 2.0 | 23.4 | 1.3 |
| F4 seeds LEF-KD3-3 | 0.6 | 16.2 | 0.3 | 3.2 | 54.4 | 2.2 | 21.7 | 1.4 |
| F4 seeds LEF-KD3-4 | 0.8 | 18.0 | 0.3 | 3.3 | 53.1 | 1.8 | 21.2 | 1.4 |
| F4 seeds LEF-KD3-5 | 0.6 | 15.6 | 0.3 | 3.3 | 54.7 | 2.5 | 21.7 | 1.3 |
| F4 seeds LEF-KD3-6 | 0.6 | 16.1 | 0.3 | 3.3 | 55.1 | 2.4 | 20.9 | 1.3 |
| F4 seeds LEF-KD3-7 | 0.6 | 16.0 | 0.3 | 3.3 | 55.8 | 1.7 | 20.8 | 1.4 |
| F4 seeds LEF-KD5-1 | 0.2 | 10.4 | 0.3 | 2.9 | 60.2 | 2.0 | 22.4 | 1.6 |
| F4 seeds LEF-KD5-2 | 0.2 | 10.8 | 0.4 | 2.8 | 60.2 | 1.8 | 22.2 | 1.6 |

Single seed

| | | | | | | | | |
|---|---|---|---|---|---|---|---|---|
| F4 seed LEF-KD1-1 #1 | 0.5 | 17.9 | 0.3 | 3.2 | 50.6 | 3.7 | 22.7 | 1.1 |
| F4 seed LEF-KD1-1 #2 | 0.5 | 18.1 | 0.4 | 3.5 | 53.2 | 3.1 | 19.8 | 1.5 |
| F4 seed LEF-KD1-1 #3 | 0.6 | 18.7 | 0.4 | 3.3 | 50.2 | 2.8 | 22.9 | 1.2 |
| F4 seed LEF-KD1-1 #4 | 0.6 | 18.5 | 0.0 | 3.6 | 49.8 | 4.7 | 21.5 | 1.4 |
| F4 seed LEF-KD1-2 #1 | 0.1 | 10.6 | 0.3 | 2.9 | 53.1 | 3.1 | 28.6 | 1.3 |
| F4 seed LEF-KD1-2 #2 | 0.1 | 10.6 | 0.4 | 3.3 | 58.2 | 4.1 | 22.0 | 1.3 |
| F4 seed LEF-KD1-2 #3 | 0.2 | 11.2 | 0.3 | 3.5 | 57.2 | 3.1 | 23.3 | 1.2 |
| F4 seed LEF-KD1-2 #4 | 0.1 | 11.5 | 0.4 | 3.5 | 57.4 | 3.1 | 22.6 | 1.3 |
| F4 seed LEF-KD1-2 #5 | 0.1 | 11.4 | 0.4 | 3.2 | 56.1 | 3.0 | 24.4 | 1.4 |
| F4 seed LEF-KD5-1 #1 | 0.2 | 10.9 | 0.4 | 3.6 | 64.5 | 3.2 | 16.0 | 1.3 |
| F4 seed LEF-KD5-1 #2 | 0.2 | 10.9 | 0.4 | 3.3 | 62.7 | 3.0 | 18.0 | 1.4 |
| F4 seed LEF-KD5-1 #3 | 0.2 | 10.3 | 0.3 | 3.3 | 63.5 | 3.0 | 17.7 | 1.6 |
| F4 seed LEF-KD5-1 #4 | 0.2 | 11.4 | 0.4 | 3.1 | 59.5 | 3.1 | 20.7 | 1.6 |
| F4 seed LEF-KD5-1 #5 | 0.2 | 12.7 | 0.4 | 2.9 | 56.6 | 2.8 | 22.6 | 1.7 |
| F4 seed LEF-KD5-2 #1 | 0.2 | 11.0 | 0.3 | 3.0 | 55.0 | 2.8 | 26.4 | 1.4 |
| F4 seed LEF-KD5-2 #2 | 0.2 | 11.3 | 0.4 | 3.2 | 59.2 | 3.2 | 20.7 | 1.9 |
| F4 seed LEF-KD5-2 #3 | 0.2 | 12.0 | 0.4 | 2.8 | 55.7 | 2.8 | 24.2 | 1.9 |
| F4 seed LEF-KD5-2 #4 | 0.2 | 11.0 | 0.4 | 3.1 | 60.1 | 2.8 | 20.9 | 1.4 |
| F4 seed LEF-KD5-2 #5 | 0.2 | 10.9 | 0.4 | 3.6 | 62.0 | 2.9 | 18.2 | 1.8 |

### Example 9—Oxidative Stability of Rice Bran and Rice Bran Oil

The accelerated rancidity testing involves GC using a sampler to detect the volatiles in the headspace of grain stored at high temperature (40° C.). A comparison of the production of volatiles, particularly hexanal, upon storage of wildtype and genetically edited plants and resulting progeny from crosses. This is an important quality issue in the rice industry for storage of grain and also of storage of rice bran.

The rice bran isolated from brown rice was passed through 0.5 mm sieve, then used for headspace analysis following an accelerated storage simulation. The vials containing 300 mg rice bran were incubated in a 37° C. oven with cap closed. The vials were removed from the oven at different time points (at day 0, 2, 4 and 8), and stored at −80° C. before HS-SPME analysis. The gas sample released from bran can be obtained in the headspace of a vial by either heating at 80° C. or by natural diffusion. The volatile components in the headspace were analysed by direct injection into a GC-MS machine (Suzuki et al., 1999). The desorption of the aroma compounds is then done thermally and the trapped molecules are analysed by GC and identified using standards. The production of hexanal from linoleic acid in vitro has been demonstrated by Nielsen et al. (2004).

The Rancimat is a standardized test to measure the oxidation stability of fats and oils by an accelerated aging process created by exposing the sample to heat and increased air volume. The time that passes until oxidation takes place is the oxidation stability index. Samples of rice were provided to the Department of Primary Industries New South Wales (DPI NSW). Rice bran oil was extracted by DPI NSW ISO659 method. About 1.8 g of rice bran oil was used for the Rancimat test. Variation in the total fatty acid composition was determined in the rice bran extracts from the gene edited mutants and stored RNAi lines (Table 11) (WO 2008/006171). The Rancimat test by DPI NSW service lab, was carried out according to the standard ISO method 110° C. airflow 20 L/hr.

TABLE 11
Total fatty acid composition of rice bran oil
extract from CRISPR mutants and RNAi lines.

| Sample | C14:0 | C16:0 | C18:0 | C18:1 | C18:2 | C18:3n3 | other |
|---|---|---|---|---|---|---|---|
| fad2-1 KD/KD + lox3KO | 0.31 | 13.12 | 1.61 | 62.43 | 19.77 | 1.04 | 1.71 |
| FAD2WT + lox3KO | 0.40 | 17.43 | 1.53 | 37.79 | 39.99 | 1.19 | 1.66 |
| NEG | 0.38 | 17.66 | 2.13 | 37.26 | 39.61 | 1.19 | 1.78 |
| FAD2-RNAi | 0.00 | 15.61 | 2.23 | 49.91 | 27.38 | 1.34 | 3.53 |
| *NEG | 0.33 | 16.87 | 2.22 | 35.62 | 39.25 | 1.55 | 4.16 |

*NEG is negative control from control line stored from prior FAD2-RNAi experiments (WO 2008/006171).

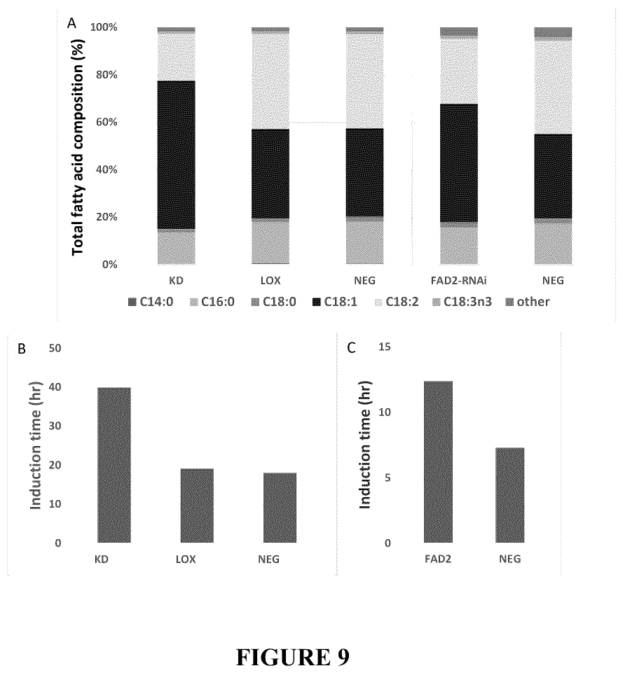

The induction time of oil is used by the edible oil industry to indicate the oxidative stability quality. As shown in FIG. 9, the oxidative stability of rice bran oil from the fad2-1 KD+lox3 KO line was measured as 39.86 hr which was more than double induction time for the negative control at 18.00 hr. The FAD2-1 WT+lox3 KO mutant line showed a slight increase of 19.09 hrs, about 1 hr greater when compared with the negative control. In comparison, the FAD2-RNAi oil induction time was 12.37 hr showed 1.7 fold increase compared with its negative control (7.28 hr). This suggested the lower percent content of C18:2 level in the rice bran oil from the fad2-1 KD+lox3 KO and FAD2-RNAi lines in the oil contributed to a higher oxidative stability in these lines compared to their respective control. In our analysis there was an 8% difference in C18:2 content between the fad2-1 KD/KD+lox3 KO and the FAD2-RNAi silenced line, which would have contributed to the variation in rice bran oil stability between the two lines. This strongly supports our conclusion that the rice bran from fad2-1 KD/KD+lox3 KO will not only have improved oil composition over the previously published FAD2-RNAi lines but offers greater stability and a longer shelf-life of the rice grain and the rice bran itself. The knockout of LOX3 in combination with the fad2-1 KD mutations may have prevented C18:2 from oxidizing after milling and during storage before oil was extracted contributing to an improved oxidative stability compared to the negative control. The reduced activity of the FAD2 mutated proteins tested here is shown in Example 10. Nevertheless, it should be noted that the RNAi and gene edited samples may not be suitable to put in to comparison directly because of the different harvesting year.

### Hexanal

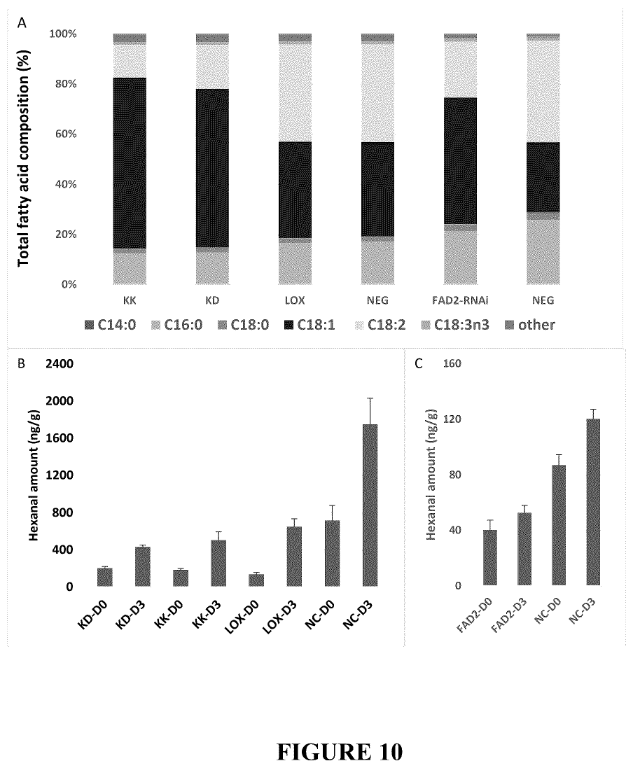

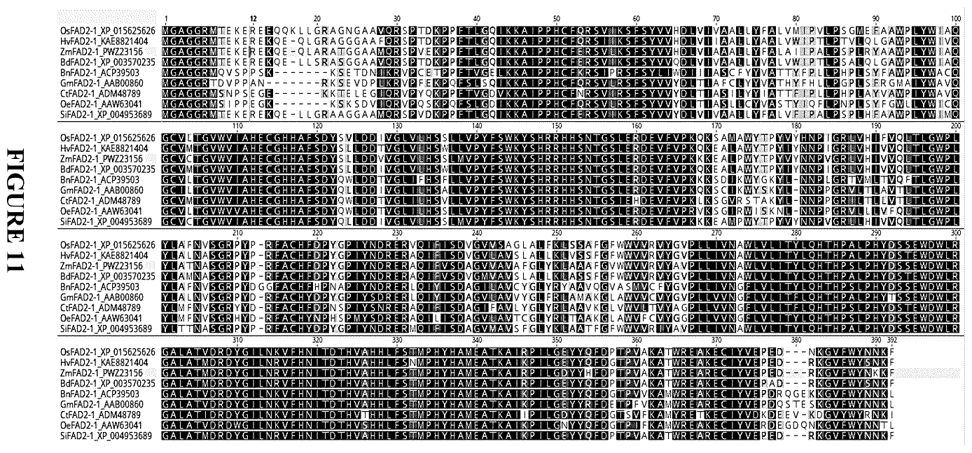

Head space result indicated the increasing of hexanal compound accumulation in each sample during a stimulated storage treatment of 3 days at 40° C. The overall amount of hexanal increased in all the samples but at a different pace and from different starting concentrations (FIG. 10).

Before the storage stimulation, the amount of hexanal was comparable between the mutant lines with fad2-1 KO/KD+lox3KO, fad2-1KD/KD+lox3KO or FAD2WT+lox3KO at about 100-200 ng/g, while the hexanal level measured was over 700 ng/g in the Negative control. This indicated the peroxidation of linoleic acid may have already started either during and or just after milling process. After the storage stimulation, the most significant increase in hexanal production was observed in the negative control with nearly 2.5 fold increase over the test period resulting in over 1700 ng/g at the conclusion of the test. The lowest amount of hexanal was approximately 400-500 ng/g in the fad2-1 KO/KD+lox3KO and fad2-1KD/KD+lox3KO. The lox3 line (i.e. FAD2WT+lox3KO) showed over 4 fold increase in the amount of hexanal produced reaching about 650 ng/g.

To compare with the amount of hexanal in the FAD2-RNAi the percent of the control was calculated. The FAD2-RNAi produced about 44% hexanal relative to its negative control line. In contrast the fad2-1 KO/KD+lox3KO and fad2-1KD/KD+lox3KO, and FAD2WT+lox3KO were 29%, 25%, and 37% of their negative control. The fact that the hexanal produced by the fad2-1 KO/KD+lox3KO and fad2-1KD/KD+lox3KO was lower than FAD2WT+lox3KO indicates that knocking out LOX3 in the high oleic acid background can further reduce the C18:2 peroxidation and the rate of peroxidation as measured by capability to produce hexanal. The lox3 mutant also possessed a lower hexanal production level on day 0. All mutants showed lower levels of hexanal accumulation at Day 3 than the Day 0 sample for the negative control. When lox3 KO was combined with reduction in expression of FAD2-1, the hexanal level can be reduced 15-20% more than simply knocking out expression of LOX3 alone.

### Example 10—Activity of Fad2 Mutants

FAD2-1 WT, fad2-1 KD and fad2-1 KD variants were synthesized and cloned into yeast expression vector pYES2 to generate pXZP1101 and pXZP1102, respectively. The plasmids were transformed into a yeast strain S288C for selection and the transformants were selected on SD-Ura (glu) plate. At least three positive single colonies of each transformant were then selected for overnight cell culture in 3 mL of the SD-Ura (glu) at 30° C. The cells were collected by centrifuging, then washed with sterile H₂O and resuspended in 3 mL of SD-Ura (gal) to OD 0.1, than inoculated to 5 mL yeast minimal media SD-Ura (gal). After for 2-3 days cell culture, the cells were harvested, washed with H₂O and freeze-dried for FAME preparation followed by GC analysis of total fatty acid composition. The conversion rate as measured by the ratio between C18:2/(C18:1+C18:2), was used to evaluate the FAD2-1 enzymatic activity in the yeast transformants. In the vector control cells, C18:2 was barely detectable showing that endogenous FAD2 was not present in S288C (Table 12). The FAD2-1 WT showed about 48% of the C18:2 conversion rate however the mutated fad2-1 KD enzyme showed reduced conversion at only 30%, nearly a 20% reduction in activity compared to the control wildtype enzyme which is approximately 60% to 62% of the wildtype. The variation of conversion rate in fad2-1 KD suggested the enzyme activity was reduced due to the 6 amino acids truncation at the N-terminus.

TABLE 12
Activity of FAD2-1 mutants experiment A.

| | C16:0 | C16:1 | C18:0 | C18:1 | C18:1d11 | C18:2 | Conversion |
|---|---|---|---|---|---|---|---|
| Sample | (%) | (%) | (%) | (%) | (%) | (%) | rate |
| Vector control | 8.67 | 23.94 | 9.00 | 56.35 | 1.27 | 0.00 | 0.00 |
| FAD2-1 WT | 22.66 | 30.61 | 11.14 | 17.11 | 2.24 | 15.87 | 0.48 |
| fad2-1 KD | 23.54 | 34.10 | 10.51 | 20.32 | 2.19 | 8.90 | 0.30 |

To identify other suitable mutants fad2-1 KD enzyme variants were expressed in the yeast system. Variants are shown in Table 13. FAD2-WT activity represents about 100% conversion in the model. As shown further mutants were shown with some conversion efficient, for example the M3 mutant has retained about 4% of the wildtype activity.

FAD2 functions as a homodimeric enzyme, expression of nonfunctional mutants of FAD2 may cause inhibition of its activity through formation of nonfunctional heterodimers. The Cas9/sgRNA constructs used in the present studies targeted the start codon and caused frame-shift mutations in the 5′ region of the targeted FAD2 genes, disrupting the N-terminal FAD2 domains. The inventors showed the reduction of activity resulting from the frame-shift mutations in the yeast model.

TABLE 13
Activity of FAD2-1 mutants

| | | | | | | | | | | C16:1->16:2 | C18:1->18:2 |
|---|---|---|---|---|---|---|---|---|---|---|---|
| Samples | C14:0 | C14:1 | C16:0 | C16:1 | C16:2 | C18:0 | C18:1 | C18:1d11 | C18:2 | conv (%) | conv (%) |
| pYES2 | 0.2 | 0.0 | 7.1 | 28.1 | 0.0 | 7.3 | 56.2 | 1.1 | 0.0 | 0.0 | 0.0 |
| control | | | | | | | | | | | |
| OsFAD2-WT | 0.6 | 0.1 | 21.1 | 29.5 | 6.1 | 9.7 | 15.7 | 1.7 | 15.7 | 17.1 | 50.0 |
| control | | | | | | | | | | | |
| OsFAD2-KD | 0.7 | 0.1 | 21.9 | 34.2 | 2.9 | 9.2 | 20.4 | 1.7 | 8.8 | 7.8 | 30.2 |
| OsFAD2-M1 | 0.9 | 0.2 | 21.7 | 42.0 | 0.0 | 7.2 | 26.3 | 1.7 | 0.0 | 0.0 | 0.0 |
| OsFAD2-M2 | 0.8 | 0.2 | 21.7 | 41.5 | 0.0 | 7.5 | 26.5 | 1.7 | 0.0 | 0.0 | 0.0 |
| OsFAD2-M3 | 0.8 | 0.2 | 21.7 | 41.2 | 0.2 | 7.6 | 26.0 | 1.7 | 0.5 | 0.6 | 2.0 |
| OsFAD2-M4 | 0.9 | 0.2 | 21.9 | 41.5 | 0.1 | 7.3 | 26.2 | 1.8 | 0.2 | 0.2 | 0.8 |
| OsFAD2-M5 | 1.0 | 0.2 | 25.2 | 38.1 | 0.0 | 8.5 | 25.3 | 1.6 | 0.2 | 0.0 | 0.8 |

In the Rice Genome Annotation Database of MSU (http://rice.plantbiology.msu.edu/index/shtml), six alternative open reading frames (ORFs) were found for FAD2-1, 4 of which started from the start codon (the 2^{nd} ATG). While ORF1 and ORF6 started from the 1st ATG in the intron and 3^{rd} ATG in the 5 end of the exon, respectively. Presumably, all of the ORFs can encode proteins of FAD2-1 isoforms. The 4 bp deletion in the fad2-1 KD allele induced a frame-shift in 5 possible ORFs but not in the 6^{th} alternative ORF. The corresponding proteins were miscoded due to the mutation and resulted in premature stop codons, aborting the translation. It is predicted that the ORF6 was not interrupted because the 4 bp deletion occurred just before the 3^{rd} ATG. The predicted ORF6 encoded a truncated enzyme, FAD2-KD, with the 6 amino acid residues removed from the N terminus. The enzyme activity of the FAD2-KD protein was reduced to about ⅔ of the wildtype enzyme FAD2-1, as shown in this Example. Therefore, the higher C18:1 content in the mutated rice lines carrying a KD mutation could be attributed to the knockout of the wildtype ORF and/or all 5 alternative ORFs, and retention of ORF6 contributed to the residue enzyme activity. Alternative editing strategies to stably reduce the expression and/or activity of the FAD2-1 could be made using the tools commonly available in the art.

The present application claims priority from AU 2021903546 filed 5 Nov. 2021, the entire contents of which are incorporated herein by reference.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Abe et al. (2018) Plant Physiol Biochem 131: 58-62.
Abdullah et al. (1986) Biotechnology 4:1087.
Barker et al. (1983) Plant Mol. Biol. 2: 235-350.
Begemann et al. (2017) Sci Rep. 7:11606.
Berger et al. (2005) Eur. J. Nutr. 44:163-173.
Bevan et al. (1983) Nucl. Acid Res. 11: 369-385.
Bumrungpert et al. (2019) J. Altern. Complement. Med. 25:353-358.
Cadwell and Joyce (199:2) PCR Methods Appl. 2:28-33.
Capecchi (1980) Cell 22:479-488.
Chapman et al. (2001) Journal of the American Oil Chemists' Society 78: 941-947.
Cheng et al. (1996) Plant Cell Rep. 15:653-657.
Clapp et al. (1993) Clin. Perinatol. 20:155-168.
Coco et al. (2001) Nature Biotechnology 19:354-359.
Coco (2002) Nature Biotechnology 20:1246-1250.
Comai et al. (2004) Plant J 37: 778-786.
Crarneri et al. (1998) Nature 391:288-291.
Curiel et al (1992) Hum. Gen. Ther. 3:147-154.
Doudna and Charpentier (2014) Science 28:346(6213):1258096.
Eggert et al. (2005) Chembiochem 6:1062-1067.
Eglitis et al. (1988) Biotechniques 6:608-614.
Feussner and Wasternack (2002) Annu Rev Plant Biol, 53: 275-97.
Fujimura et al. (1985) Plant Tissue Cultural Letters 2:74.
Garfinkel et al. (1983) Cell 27: 143-153.
Graham et al. (1973) Virology 54:536-539.
Grant et al. (1995) Plant Cell Rep. 15:254-258.
Greve (1983) J. Mol. Appl. Genet. 1:499-511.
Ha (2005) Nutrition research 25:597-606.
Harayama (1998) Trends Biotechnol. 16:76-82.
Haft et al. (2005) Computational Biology, PLoS Comput Biol 1(6):e60.
He and Ding (2020) Frontiers in Plant Science, 11: 562785-85.
Hellinga (1997) Proc. Natl. Acad. Sci. 94:10015-10017.
Henikoff et al. (2004) Plant Physiol 135: 630-636.
Hinchee et al. (1988) Biotech. 6:915.
Ida et al. (1983) Agricultural and Biological Chemistry 47: 637-41.
Jezequek et al. (2008) Biotechniques 45:523-532.
Jinek et al. (2012) Science 337:816-821.
Joshi (1987) Nucl. Acid Res. 15: 6643-6653.
Juliano (1985) Rice: Chemistry and Technology (American Association of Cereal Chemists).
Langridge et al. (2001) Aust. J. Agric. Res. 52: 1043-1077.
Lemieux (2000) Current Genomics 1: 301-311.
Leung et al. (1989) Technique 1:11-15.
Liang et al. (2017) Nat Commun. 8:14261.
Liang et al. (2018) Plant Biotechnol J. 16:2053-2062.
Liang et al. (2019) Methods Mol Biol. 1917:327-335.
Liu et al. (2019) BMC Plant Biology, 19: 393-93.
Lu et al. (1993) J. Exp. Med. 178: 2089-2096.
Luo et al. (2016) Plant Cell Rep 35(7):1439-1450.
Ma et al. (2015) Mol Plant, 8: 1274-84.
Makarova (2015) Nat. Rev. Microbiol. 13:722-736.
Mayer and Shanklin (2005) Journal of Biological Chemistry, 280: 3621-27.
Medberry et al. (1992) Plant Cell 4: 185-192.
Medberry et al. (1993) Plant J. 3: 619-626.
Mizuno et al. (2003) Plant Cell Physiol, 44: 1168-75.
Moghadasian and Frohlich (1999) Am. J. Med. 107: 5 88-94.
Morineau et al. (2017) Plant Biotechnol. J. 15:729-739.
Morrison (1988) J Cereal Sci. 8:1-15.
Most et al. (2005) Am J Clin Nutr 81:64-8.
Needleman and Wunsch (1970) J. Mol Biol. 45:443-453.
Ness et al. (2002) Nature Biotechnology 20:1251-1255.
Newcomer and Brash (2015) Protein Sci. 24:298-309.
Nielsen et al. (2004) Journal of Agricultural and Food Chemistry 52:23 15-232 1.
Ohta et al. (1986) Plant Cell Physiol. 27:911-918.
Ostermeier et al. (1999) Nature Biotechnology 17:1205-1209.
Ouyang et al. (2007) Nucleic Acids Research, 35: D883-D87.
Ow et al. (1986) Science 234: 856-859.
Pal and Pratap (2017) J. Oleo. Sci. 66: 551-556.
Prasher et al. (1985) Biochem. Biophys. Res. Comm. 126: 1259-68.
RoyChowdury et al. (2016) Biomed. Res. Int. doi: 10.1155/2016/4275904. Epub 2016 Jun. 15.
Rukmini and Raghuram (1991) J. Am. Coll. Nutr 10:593-601.
Salomon et al. (1984) EMBO J. 3: 141-146.
Shanklin et al. (1998) Annu. Rev. Physiol. Plant Mol. Biol. 49:611-641.
Shi et al. (2015) Trends in Plant Science, 20: 741-53.
Sieber et al. (2001) Nature Biotechnology 19:456-460.
Slade and Knauf (2005) Transgenic Res. 14: 109-115.
Stalker et al. (1988) Science 242:419-423.
Stemmer (1994a) Proc. Natl. Acad. Sci. USA 91:10747-10751.
Stemmer (1994b) Nature 370(6488):389-391.
Stoutjesdijk et al. (2002) Plant Physiol 129: 1723-1731.
Sun et al. (2016) Molecular Plant 9: 628-631.
Suzuki et al. (1993) J Agric Food Chem, 47: 1119-24.
Suzuki et al. (1999) J Agric Food Chem, 47: 1119-24.
Suzuki and Matsukura (1997) Plant Science, 125: 119-26.
Svitashev et al. (2016) Nat Commun. 7:13274.
Theriault et al. (1999) Clin. Biochem. 32: 309-19.
Thillet et al. (1988) J. Biol. Chem. 263:12500.
Toki et al. (2006) The Plant Journal, 47: 969-76.
Toriyama et al. (1986) Theor. Appl. Genet. 205:34.
Umate (2011) Plant Signaling & Behavior, 6: 335-38.
Volkov et al. (1999) Nucleic Acids Research 27:e18.
Wagner et al. (1992) Proc. Natl. Acad. Sci. USA 89:6099-6103.
Woo et al. (2015) Nat Biotechnol. 33:1162-1164.
Xu et al. (2015) Plant Biotechnol J. 13:526-539.
Yuan et al. (1996) J Biol Chem, 271: 11034a-34.
Zafar et al. (2020) J. Exp. Botany 71:470-479.
Zaplin et al. (2013) Functional Plant Biology 40: 996-1004.
Zhao et al. (1998) Nature Biotechnology 16:258-261.
Zhou et al. (2011) J. Biol. Chem. 286:43644-43650.

1. Fertile cereal grain comprising a genetically modified FAD2-1 gene and a genetically modified LOX3 gene, wherein the grain comprises
i) at least some FAD 2-1 protein activity, wherein the FAD2-1 activity is reduced when compared to a wild type cereal grain, and
ii) reduced LOX3 protein activity when compared to the wild type cereal grain.

2. The grain of claim 1 which is rice grain.

3. (canceled)

4. The grain of claim 1, wherein the grain has a total fatty acid content comprising between 50% and 80%, between 55% and 75%, between 55% and 70%, oleic acid (w/w dry weight).

5. The grain of claim 1, wherein the grain has a total fatty acid content comprising less than 15%, between 15% and 22% or between 15% and 21%, palmitic acid (w/w dry weight).

6. The grain of claim 1, wherein the grain has a total fatty acid content comprising less than 20%, less than 15%, less than 10%, less than 5%, between 2% and 20% or between 5% and 15%, linoleic acid (w/w dry weight).

7. The grain of claim 1, wherein the grain has no LOX3 protein activity.

8. (canceled)

9. The grain of claim 1, wherein the genetic modification of the LOX3 gene is a premature stop codon in the LOX3 gene.

10-12. (canceled)

13. The grain of claim 1, wherein the genetically modified FAD2-1 gene encodes a mutant FAD2-1 protein.

14-19. (canceled)

20. Cereal bran comprising genetically modified cells comprising
i) at least some FAD 2-1 protein activity, wherein the FAD2-1 activity is reduced when compared to a wild type cereal bran, and
ii) reduced LOX3 protein activity when compared to the wild type cereal bran.

21. (canceled)

22. Extracted cereal grain oil, or cereal bran oil, having a total fatty acid content comprising between 50% and 80%, or between 55% and 80%, oleic acid (w/w dry weight), and having an induction time of at least 25 hours as measured by Rancimat test conducted at 110° C. at an airflow rate of 20 L/hr, and/or which is more stable than cereal oil extracted from a cereal grain or bran lacking i) and ii) of claim 1.

23-32. (canceled)

33. A cell, preferably a rice cell, which comprises the genetic modifications as defined in claim 1, or an exogenous polynucleotide encoding a mutant FAD 2-1 protein which has between 5% and 95% less, between 20% and 80% less, between 40% and 70% less, or between 50% and 60% less, Δ12 desaturase activity than a corresponding wild type FAD2-1 protein.

34-36. (canceled)

37. A fertile cereal plant, preferably a rice plant, comprising one or more of cereal grain of claim 1, bran of the rice grain, a mutant FAD 2-1 protein which has between 5% and 95% less, between 20% and 80% less, between 40% and 70% less, or between 50% and 60% less, Δ12 desaturase activity than a corresponding wild type FAD2-1 protein, a polynucleotide encoding the mutant FAD 2-1, a vector comprising the polynucleotide, or a cell comprising the polynucleotide or the vector.

38-40. (canceled)

41. A method of producing a genetically modified cereal plant, the method comprising
i) introducing a genetic modification into a cereal cell such that it encodes a mutant FAD 2-1 protein which has between 5% and 95% less, between 20% and 80% less, between 40% and 70% less, or between 50% and 60% less, Δ12 desaturase activity than a corresponding wild type FAD2-1 protein, and
ii) producing a plant from the cell.

42-43. (canceled)

44. The method of claim 41, wherein
i) the cell does not encode a functional LOX3 protein, or
ii) the method further comprising introducing a genetic modification such that the plant, or a descendent thereof, does not encode a functional LOX3 protein in its grain and/or bran.

45-49. (canceled)

50. A method for identifying a cereal plant of claim 37, the method comprising the steps of
i) obtaining a nucleic acid sample from a cereal plant, and
ii) screening the sample for the presence or absence of a first genetic modification which reduces but does not abolish FAD 2-1 protein activity in grain of a plant when compared to a wild type cereal grain, and a second genetic modification which reduces LOX3 protein activity in grain of the plant when compared to a wild type cereal grain.

51. A process of producing extracted cereal grain and/or cereal bran oil, the process comprising;
i) obtaining grain of claim 1 from a cereal plant, or bran from the grain, and
ii) extracting oil from the grain and/or cereal bran.

52. (canceled)

53. A method of producing a cereal plant part, the method comprising,
a) growing a cereal plant, or at least 100 such cereal plants in a field, claim 37, and
b) harvesting the cereal plant part from the cereal plant or cereal plants.

54. (canceled)

55. A method of producing cereal flour, bran, wholemeal, malt, starch or oil obtained from grain, the method comprising;
a) obtaining the grain of claim 1, and
b) processing the grain to produce the flour, bran, wholemeal, malt, starch or oil.

56-57. (canceled)

58. A product produced from the grain of claim 1 or bran from the grain.

59-60. (canceled)

61. A method of preparing a food ingredient, beverage ingredient, a food product, or a beverage product, the method comprising processing grain of claim 1, or bran, flour, wholemeal, malt, starch or oil from the grain, to produce the food ingredient, beverage ingredient, food product, or beverage product.

62-65. (canceled)
